(19) United States
(12) Patent Application Publication
(10) Pub. No.: US 2025/0042733 A1
(43) Pub. Date: Feb. 6, 2025

(54) METHOD OF HYDROGEN MANUFACTURE

(71) Applicant: Arrowhead Center, Inc., Las Cruces, NM (US)

(72) Inventors: Huiyao Wang, Las Cruces, NM (US); Edirisooriya Mudiyanselage Nimanthi Thiloka Edirisooriya, Las Cruces, NM (US); Pei Xu, Las Cruces, NM (US)

(73) Assignee: Arrowhead Center, Inc., Las Cruces, NM (US)

(21) Appl. No.: 18/378,586

(22) Filed: Oct. 10, 2023

Related U.S. Application Data
(60) Provisional application No. 63/414,332, filed on Oct. 7, 2022.

(51) Int. Cl. C01B 3/32; B01J 21/06; B01J 23/06; B01J 23/42; B01J 23/52; B01J 35/23; B01J 35/39; B01J 37/02; C07C 51/347; C07C 51/41; C08J 11/14

(52) U.S. Cl. CPC C01B 3/326; B01J 21/063; B01J 23/06; B01J 23/42; B01J 23/52; B01J 35/23; B01J 35/39; B01J 37/0221; C07C 51/347; C07C 51/41; C08J 11/14; C01B 2203/0227; C01B 2203/107; C01B 2203/1082; C01B 2203/1211; C01B 2203/1258; C08J 2367/02

(57) ABSTRACT

A composition for producing hydrogen and degrading polymers. The composition comprises a semiconductor and individual metal atoms/clusters attached to the semiconductor. A method for producing hydrogen. The method comprises pretreating a polymer, contacting polymer with composition for producing hydrogen exposing the composition for producing hydrogen to radiation, degrading the polymer, and releasing hydrogen. The radiation may be solar radiation. Pretreatment of the polymer may comprise contact with an acid, a base, and/or ethanol. The pretreatment may occur in the absence of light.

### CROSS-REFERENCE TO RELATED APPLICATIONS

This application claims priority to and the benefit of the filing of U.S. Provisional Patent Application No. 63/414,332, entitled “METHOD OF HYDROGEN MANUFACTURE”, filed on Oct. 7, 2022, and the specification and claims thereof are incorporated herein by reference.

### BACKGROUND OF THE INVENTION

### Field of the Invention (Technical Field)

The present invention relates to compositions and methods for the degradation of polymers and hydrogen production.

### Description of Related Art

Global energy consumption has been increasing rapidly in recent decades due to population and economic growth. According to recent statistics, ˜60% of the U.S. energy requirement is satisfied by fossil fuel reserves such as natural gas and coal, which amplifies greenhouse gas emissions. However, global annual greenhouse gas emissions are targeted to be reduced by 85% by 2050. Even though the conventional eco-friendly renewable energy methods (e.g., wind, thermal, sunlight, and hydropower) contribute to the overall energy demand in the U.S., those methods cannot act as the replacement for fossil fuel production due to the high production and maintenance costs along with the limited supply.

Clean energy has been the focus of significant research with hydrogen being identified as a green alternative energy source. There are several methods of producing hydrogen including electrolysis, thermal processes, biological methods, and photocatalytic processes that use domestic resources like natural gas, biomass, and renewable resources such as wind and solar power.

Hydrogen (“H₂”) is a valuable alternative energy source to the increasing energy demand. Hydrogen is identified as a key contributor to future energy systems due to its production capability using renewable energy sources with zero greenhouse gas emissions. With the increasing global warming and climate changes, the green hydrogen economy can be beneficial in terms of its wide application in power generation, transportation, industry, building heating, and energy storage. The current natural gas leakage from the distributed system for the U.S. is estimated as 0.69 Tg-2.9 Tg CH₄/year, and it is predicted that replacing natural gas with hydrogen could substantially reduce global warming.

Renewable energy sources such as solar power, biomass, wind, geothermal, and hydropower are used to generate H₂ under different technologies. Photonic energy, biochemical energy, thermal energy, and electric energy generated from renewable sources are applied in H₂ production systems. Photonic energy from sunlight is applied in photocatalytic and photo-reforming H₂ generation processes. Biochemical energy generated utilizing biomass and solar power can be applied in H₂ generation by enzymatic, metabolic, anaerobic, and fermentation processes. H₂ production from thermochemical, thermolysis, and thermochemical gasification processes are powered by thermal energy generated by solar energy. The electrical energy generated by renewable sources such as wind, geothermal, hydro, and solar power can be applied in H₂ generation systems such as electrolysis, H₂S cracking, and hybrid thermo-chemical systems.

The most common hydrogen production method is steam reforming of hydrocarbons (methane in natural gas or coal) which is considered grey hydrogen and emits high carbon dioxide amounts to the environment. Blue hydrogen is introduced after applying carbon capture and storage or carbon capture and utilizing technologies to lower greenhouse gas emissions. Hydrogen can also be generated by electrolysis of water, photolysis of water, and thermolysis processes. When such technologies use the energy generated by clean, renewable sources, such as hydro, wind, or solar, the hydrogen is termed “green hydrogen”. Additionally, hydrogen produced using nuclear energy is defined as purple hydrogen, and there are other forms of hydrogen produced using thermochemical processes due to the high temperatures of the nuclear reactor. The turquoise hydrogen is generated by splitting hydrocarbons. The plasma process used for producing carbon black and hydrogen is the most developed turquoise hydrogen production method, and there are other techniques, such as cold plasma, methane catalytic conversion, and molten metal pyrolysis via thermal splitting.

Water electrolysis is categorized into alkaline water electrolysis, proton exchange membrane electrolysis, solid oxide water electrolysis, and alkaline anion exchange membrane water electrolysis. Photocatalysis is considered an attractive process of hydrogen production as it converts solar energy into chemical energy. Photocatalysis is generally divided into up-hill reactions and down-hill reactions, where in down-hill reactions, photon energy absorbed by photocatalysis is used to induce thermodynamically favored reactions, while in up-hill reactions, photon energy is converted into chemical energy. Water splitting into hydrogen and oxygen falls under up-hill reactions (where ΔG⁰=237 KJ/mol corresponds to 1.23 eV), and is further divided into three groups, namely thermochemical water splitting, photobiological water splitting, and photocatalytic water splitting.

Solar energy is the most significant renewable energy source that can be used in green hydrogen production. Solar thermal, solar photovoltaic, and photoelectrochemical energy are the three primary forms of solar energy that can be utilized as energy sources in hydrogen production systems. The solar spectrum is comprised of <5% ultraviolet (“UV”) light, ˜45% visible light, and ˜50% infrared (“IR”) light. It is desired to extend the light absorbance of catalysts from the UV to visible or IR region to achieve improved solar light utilization. The bandgap of a photocatalyst determines the range of light wavelengths it can absorb. Bandgap engineering is performed to match the bandgap of the catalyst with the solar spectrum. Surface area and morphology, incorporation of cocatalysts such as noble metals, and carbon dots, application of tandem catalysts, incorporation of quantum dots, and surface passivation are considered in the bandgap engineering.

As an energy conversion technology, photocatalytic water splitting using sunlight provides an efficient and promising method. The semiconductor photocatalysis process begins with photon absorption and consequently converts solar energy into chemical fuels. The irradiation of semiconductor particles with incident light of an equal or greater energy than the bandgap of the material excites the valence band (“VB”) electrons to the conductive band (“CB”), a phenomenon called optical excitation. Photo-reforming is a process that harnesses the redox ability of photocatalysts upon illumination, to simultaneously drive the reduction of hydrogen ion (“H⁺”) into hydrogen gas and the oxidation of organic compounds. Photo-reforming of plastic waste has been identified as an efficient method of recovering H₂. Photo-reforming requires four components such as photocatalyst, substrate, sunlight, and water. An additional step is required to recover catalyst from the suspended photoreactive solutions. However, the hybrid photocatalytic membrane process does not require a complicated recovery of photocatalysts after water treatment. This hybrid technology uses stationary nanostructured photocatalysts to enhance the absorption of photons and reactants so that the catalyst does not need to be suspended in solution. In the process of converting solar energy into chemical fuels, the photocatalyst should follow the following sequential main process.

The sun emits radiation ranging from X-rays to radio waves, but the most intense solar radiation occurs in the visible light range (43% of the solar energy reaching the earth's surface is from 400 to 700 nm). Therefore, it is crucial to focus on photocatalysts that can harvest ultraviolet and visible light. When a photon hits the photocatalyst, an electron-hole pair is generated, and the electron moves from the VB to CB. For a photocatalyst to split water and generate hydrogen, it should have an appropriate bandgap and properly located conduction bands and valence bands for oxidation/reduction reactions, and an effective electron-hole pairs separation. The instability caused by the charge pair separation tends to influence the recombination of the electron-hole. The electron-hole recombination can occur at the surface of the catalyst. Hence, the photocatalyst should undergo the charge transport efficiently and effectively to the surface of the catalyst. In the CB site, the H⁺ extracts the electron and forms an H radical where the site undergoes a further termination reaction by producing H₂. The negatively charged microplastics undergo the oxidation process by transferring the charged moiety to the catalyst, thereby completing the photo-reforming cycle.

Photo-reforming is a process to generate green H₂ using the redox ability of photocatalysts under illumination. During photo-reforming, photogenerated electrons are used to reduce aqueous H⁺ ions to produce H₂, while generated holes (the vacancy created due to the electron movement from valance to conduction band) trigger oxidation reactions of organic compounds or biomass. One of the significant advantages of photo-reforming reactions is that the catalyst could have a relatively lower valance band energy depending on the organic oxidation reaction pathways despite the bandgap energy to reduce H⁺ and water oxidization. In contrast, water splitting requires a photocatalyst with sufficient bandgap to energize the abovementioned reactions. This opens a variety of options when selecting the catalyst, including the tunable to the highest intensity natural light frequency, and cost-effective composite catalyst. Additionally, photo-reforming reactions can form superoxide radicals that compete with the H⁺ reduction reaction in an oxygen-rich environment. In an anoxic environment, oxidation reactions can happen in two pathways, including direct hole transfer and the formation of highly oxidizing hydroxyl radicals. Photo-reforming offers the potential to both provide a supply of H₂ and to remove and degrade plastic. Photo-reforming of common plastic wastes, such as polyethylene terephthalate (PET), in an alkaline slurry system, produces green hydrogen using renewable energy and without greenhouse gas emissions.

Photo-reforming efficiency can be optimized by changing the process conditions to increase hydrogen generation. The concentration of dissolved organic matter, reaction temperature, pH of the reactants, reactor configuration (slurry type, packed bed, or thin film), reactor material, irradiation wavelength, and more importantly, an appropriate catalyst with suitable active sites and chemical properties, are main factors that affect the efficiency of hydrogen production. Dissolved organic concentration can be correlated to the kinetics of the reaction oxidation pathway by the oxidation selectivity. Furthermore, the organic concentration in the solution regulates the adsorption capacity of the catalyst. The reaction temperature and contact time are other important parameters that influence the reaction efficiency by regulating the stability of the intermediate species, determining the residence time of adsorbed species remaining on the catalyst surface, and thereby defining the selectivity towards different reaction pathways. The pH of the reaction, which defines the relative charge of the hydrated species, can impact the reaction rate and selectivity towards oxidation products. However, changing the pH will make the reaction selectivity more complex due to the effect on band potentials, adsorption, desorption kinetics, and physicochemical properties.

When designing the reactors for photo-reforming, the adsorption of reactant species onto the catalyst surface has also been shown to influence the efficiency of hydrogen production. The sequence of photocatalytic reactions is initiated when reactants and transient species are adsorbed on the catalyst surface under light irradiation. The photo-absorption, charge separation, and surface charge consumption efficacies are defining factors of the reaction efficiency, which can be optimized by designing the reactor to achieve optimum light exposure while providing an efficient mass transport facility. Hence, the incident photon flux, photon energy, and the light source are also important parameters. Reactor material, its size, dimension, and shape are some other considerations when designing a reactor because the irradiation wavelength absorbed at the catalyst surface may depend on the reactor material as the intermediate medium between the illumination source and the catalyst surface. Hence, quartz, pyrex, and vycor have been previously tested and considered favorable reactor materials.

Understanding the important design parameters of the photo-reforming system assists in addressing the challenges in the hydrogen production system, such as potential competition between H₂O oxidation and organic oxidation, the requirement of constant separation of targeted products to prevent overoxidation into undesirable products, and the complexity of adsorption and desorption of reactant, intermediate, and product species.

The removal and degradation of plastic in water pose an environmental challenge. A significant amount of plastics accumulates in the food chain and causes adverse effects on living organisms. Membrane bioreactors, retro filtration, bacterial oxidation, rapid sand filter, disc filter, coagulation process, and magnetic extraction are currently used for plastic removal from water. However, a much greener approach is required for removing and degrading plastics in water.

Photodegradation of polymers is a process of breaking the polymeric chain by absorption of photons induced by the light source. Polymer undergoes both physical and chemical changes through this process. Therefore, activation energy is of prime importance in photodegradation. Specific polymers absorb a certain wavelength at which they become activated when photo-exposed.

The plastics released into the natural water bodies undergo embrittlement, fragmentation, and photodegradation. It takes many years to form microplastics and nano plastics. The microplastics (less than 5 mm) are mainly categorized into primary and secondary microplastics. The primary and secondary microplastics can be further categorized based on the shapes (fibers, films, foams, foils, fragments, pellets, and spheres), the composition, and the chemical composition (acrylic polyamide, polyester, high- and low-density polyethylene (“HD/LD-PE”), polyethylene terephthalate (“PET”), polypropylene (“PP”), polystyrene (“PS”), and polyvinyl chloride (“PVC”)).

The primary microplastics are the raw materials of plastics while the fragmentation of larger plastics due to degradation are considered as secondary microplastics. The plastic microbeads in facial and body cleansers, and synthetic fibers in air and water created through abrasion are the primary sources of microplastics. Further, paints and tire dust are the most common sources that produce tiny plastics, and they release into waterways. Several studies reported that sewage treatment plants are significant sources of microplastics and the sludge or biosolid applications in agricultural systems present a major contribution to microplastics in the environment.

Plastics within the range of 1 nm to 1000 nm are defined as nano plastics that formed because of the breakdown and degradation of microplastics. These particles show a colloidal behavior in water with highly polydispersed physical properties and heterogeneous composition. Dispersion and aggregation of nano-plastics are influenced by the solution chemistry parameters such as pH, divalent cations, and natural organic matter. The nano-sized plastics have higher surface curvature, surface area, and relatively small surface structures which alters their chemical and biological interactions. The main properties that define nano-plastics are summarized in Table 1.

TABLE 1
Properties of nano-plastics

| Property | Description |
|---|---|
| Composition | Mixture of polymers, highly weathered |
| Size | 1 nm-1 μm |
| Size distribution | Polydisperse (continuum of size) |
| Shape | Asymmetrical |
| Surface charge | Uncontrolled with the presence of |
| | different species |
| Stability | Hetero aggregation with other natural |
| | and/or anthropogenic colloids during |
| | the formation process |
| | Depending on both nano-plastics and |
| | their surrounding media physical and |
| | chemical properties |
| Porosity | Open structure |

Translocation of microplastics (0.2 mm and 150 mm) across the gastrointestinal tract in the mammalian gut and into the lymphatic system has been demonstrated in studies involving humans. The physical presence of microplastics may be toxic due to their inherent ability to induce intestinal blockage or tissue abrasion. In earthworms (Eisenia ndrei), fibrosis, congestion, and inflammatory infiltrates were observed after exposure to microplastics (from 62 mg/kg to 1000 mg/kg of PE). In sea bass (Dicentrarchus labrax), moderate to severe histopathological alterations of the intestine were measured, after 30 days to 90 days of exposure to PVC microplastic through ingestion. The health impacts can vary with the types of plastics (Table 2).

TABLE 2
Potential health impacts from different microplastics

| Plastic | Potential health impact |
|---|---|
| Polyethylene terephthalate (PET): durable | Can leach antimony in amounts exceeding |
| plastic used in bottles for water, juice, and | U.S. safety guidelines when exposed to high |
| soda. | temperature. |
| High density polyethylene (HDPE): used | Leach estrogenic chemicals when exposed |
| to package milk, juice, water, cleaning | to heat, boiling water, and sunlight, which |
| supplies, and shampoos. | cause breast cancer, endometriosis, altered |
| | sex ratios, testicular cancer, poor semen |
| | quality, early puberty, and malformations of |
| | the reproductive tract. |
| Polyvinyl chloride (PVC): wraps meat and | Leach toxic chemicals when in contact with |
| sandwiches, floats in the tub in the form of | water. Four chemical softeners used with |
| bath toys, and makes for stylish jackets and | PVC. These chemicals interfere with the |
| household plumbing. | body's production of hormones. |
| Low density polyethylene (LDPE): used in | LDPE is considered a low hazard plastic. |
| shrink wrap, milk cartons, take out coffee | |
| cups and to package sliced bread, | |
| newspapers, and dry cleaning. | |
| Polypropylene (PP): used in containers for | Fairly safe plastic. |
| yogurt, takeout lunches, medicine, and | |
| syrups. | |

Toxicity studies of nano-plastics suggest that acute toxicity and physical damage to Daphnia Magna are associated with the solution chemistry parameters and the particle surface modification. Further, the more complex the solution conditions are, the more toxicity the plastic nanoparticle can cause.

A recorded amount of plastics (4.8 to 12.7 million metric tons in 2010) contaminating the sea from the total production of plastics (275 million metric tons in 2010), and the environmental pollution will increase with population due to the broad applications for plastics and the massive amounts of plastic-contained products. Research on the South African coast suggests that the average concentration of microplastics in sea trawls can be 4.01±3.28 plastics/100 m². When considering freshwater, three major contamination routes are identified, namely, effluent discharged from wastewater treatment plants, overflow of wastewater sewers during high rain events, and runoff from sludge applied to agricultural land. Due to the ineffective removal of plastics during sewage treatment, it is found that 15,000 to 4.5 million microplastics are released into the environment daily.

There is direct evidence that nano-plastics can accumulate in plants, depending on their surface charge. The positively charged nano plastics can accumulate at relatively low levels in the root tips, but these nano-plastics were found to induce a higher accumulation of reactive oxygen species and inhibit plant growth and seedling development more strongly than negatively charged sulfonic-acid-modified nano-plastics. By contrast, the negatively charged nano plastics were observed frequently in the apoplast and xylem. Plant accumulation of nano-plastics can have both direct ecological effects and implications for agricultural sustainability and food safety.

The removal of microplastics from aqueous media has been studied using different technologies such as membrane bioreactors, retro filtration, and bacterial oxidation. Final stage wastewater treatment techniques such as membrane bioreactor, rapid sand filter, and disc filter were studied to identify their suitability to remove microplastics, and treatment efficiencies were reported as 99.9%, 97%, and 95% for membrane bioreactor, rapid sand filter, and disc filter respectively. Filtration was found to be unsuccessful due to clogging and the isolation of particles. Acidic digestion also failed due to excessive solid loads.

The coagulation process, such as Al-based coagulants, was found to be more effective in removing microplastics than Fe-based coagulants. Ionic strength, the concentration of natural organic matter, and turbidity level showed a lower influence on removal efficiency. However, membrane fouling was induced after coagulation with Al-based salts at a conventional dosage, especially for the large particle size. The removal of microplastics by coagulation and ultrafiltration processes indicates application potential for drinking water treatment.

Photocatalytic H₂ production technologies have advanced in the last decades. Among the different ways to modify the photocatalyst, namely dye sensitizing, doping, semiconductor compounding, and noble metal deposition, noble metal deposition has attracted considerable attention due to its unique characteristics that greatly improve the photocatalytic properties of semiconductor materials. Metal dopants such as Au and Pt can boost the H₂ production efficiency of TiO₂ through Schottky barrier formation, surface plasmon resonance, and generation of gap states by interaction with TiO₂ valance band states.

The main groups of photocatalysts used for photocatalytic H₂ production technologies are titanium dioxide (“TiO₂”), cadmium sulfide (“CdS”), zinc oxide/sulfide (“ZnO/ZnS”), and other metal oxide-based photocatalysts. Gold-cadmium sulfide (“Au—CdS”) has the best performance among individual photocatalysts when considering the bandgap, quantum yield, and H₂ production rates. However, low light absorption range, electron-hole recombination, slow oxidation, and reduction kinetics, and photo-corrosion are the drawbacks of metal-based photochemical devices. Some semiconductor materials act as photocatalysts, and their properties are shown in Table 3.

TABLE 3
Different semiconductor materials as
photocatalysts and their properties

| Semi- | |
|---|---|
| conductor | Properties |
| TiO₂ | Low production cost |
| | Good chemical stability |
| | Application of TiO₂ using solar energy is highly restricted |
| | by its large bandgap (3.2 eV) and low quantum efficiency |
| Zinc | n-type semiconductor |
| oxide | Higher absorption efficiency across a large fraction of the |
| (“ZnO”) | solars pectrum when compared to TiO₂. |
| | Nonstoichiometric of ZnO rendering it a better |
| | photocatalyst compared to TiO₂ under solar irradiation. |
| | Large excitation binding energy (60 meV) |
| | Deep violet/borderline UV absorption at room temperature |
| | Antifouling and antibacterial properties |
| | The main drawback for ZnO semiconductors as |
| | photocatalysts is their low-charge separation efficiency. |

Doping is a method to enhance the light absorption spectrum to the visible region for wide bandgap semiconductors and improve their photocatalytic activity. Doping ions are functional in modifying the electronic and optical properties of semiconductors and improving conductivity and charge carriers transport and separation. Metals with very high work function (e.g., platinum (“Pt”), gold (“Au”), palladium (“Pd”)), generally showed better performance as a TiO₂ co-catalyst, due to efficient utilization of electrons in the co-catalytic sites. However, high quantities of doping elements could act as recombination centers for photogenerated charges that limit photocatalytic activity.

TiO₂ is a prominent material as a photocatalyst because of its high photocatalytic activity, non-toxicity, and biological and chemical inertness. The larger bandgap value (3.2 eV) gives numerous advantages in terms of photocatalytic water splitting. The TiO₂ has many polymorphs including anatase, rutile, and brookite. The anatase phase is more photoactive than rutile, but when they are used in combination with specific ratios, the activity is enhanced.

However, TiO₂ alone is inadequate for optimal photocatalytic activity due to the fast recombination rates of charge carriers generated at the conduction and valence bands of TiO₂ upon photoexcitation. In terms of hydrogen production rate per gram catalyst, gold-titanium oxide (“Au—TiO₂”) has the highest rate and the introduction of Au nanoparticles dispersed onto TiO₂ has been found to increase reaction efficiency by facilitating electron transfer. Therefore, doping Au into the TiO₂ matrix can inhibit the electron-hole recombination and reduce the overpotentials for H₂ generation.

The photocatalytic reforming of organics highly depends on the ability of some organic species to donate electrons to the positive holes of the illuminated photocatalyst and be oxidized, generating proton ions, while photogenerated electrons reduce the latter to produce H₂. The VB and CB of TiO₂ are bent when a noble metal is added because of the formation of a Schottky barrier, arising from the difference in the Fermi levels between the metal and the semiconductor. The higher the work function, the greater the Schottky barrier in the metal-semiconductor heterojunction, resulting in a more efficient charge separation, which is a critical step in photocatalytic processes.

The selection of a proper hole scavenger (sacrificial agent) is crucial in the photo-reforming process since the organic oxidation pathway influences the energy barrier of the reaction. The most common hole scavengers are methanol, ethanol, and glycerol, and biomacromolecules such as monosaccharides, disaccharides, and polysaccharides are used in successful hydrogen production via photo-reforming. Despite higher structure stability and low solubility, carbohydrates, lignin, food waste, cardboard, paper, and plastic waste have also been tested as sacrificial agents for hydrogen production.

Adding organic compounds in the photocatalytic system enhanced the H₂ evolution efficiency by contributing to the redox reactions. The organic compounds (sacrificial agents or hole scavengers) act as reducing agents (electron donors) and utilize the photogenerated holes, thus oxidizing similarly to the oxygen generation process in the water-splitting process while promoting H₂ generation efficiency. The sacrificial agent can reduce the carbon to hydroxide (“C:OH”) ratio and enhance the photocatalytic activity of the photo-reforming process. Properties of the sacrificial agent, such as polarity and oxidation potential, as well as the surface properties of the catalyst, adsorption, and electron donation ability, affect H₂ production efficiency.

In general, photo-reforming of plastics shows lower H₂ production due to the polymeric nature of long chain structure, low water solubility, and poor biodegradability. Polyethylene, polystyrene, and isoprene rubber have been reported to produce 25.0 μmol, 19.4 μmol, and 36.7 μmol H₂ per gram catalyst per hour (“μmolg_{cat}⁻¹ h⁻¹”), respectively, with 10 M NaOH at 70° C. using Pt 0.5 (w/w) % deposited CdOₓ/CdS/SiC composite materials. Furthermore, polyethylene terephthalate and polylactic acid were used as sacrificial agents to produce 111 μmol H₂ per gram of substrate (“μmolgsub⁻¹”) and 211 μmolgsub⁻¹ of H₂, respectively, using CNx/Ni₂P catalyst. Despite low H₂ production compared to more easily degradable organic compounds, utilizing waste plastics as sacrificial agents is a cost-effective, versatile, and environmentally friendly option. However, using plastics as sacrificial agents has the drawbacks of limited stability, potential side reactions, requirement of additional pretreatment, and difficulty in separation after the reaction.

The plastic degradation process is important when using them as sacrificial agents because their effect on the efficiency and selectivity of the reaction influences the photo-reforming process. There are several methods of plastic pretreatment, such as hydrolysis, alcoholysis, aminolysis, methanolysis, glycolysis, and pyrolysis, which vary by the reagent and technique used in the process. Among the pretreatment processes, hydrolysis is the widely applied method that can perform under mild conditions and tolerate highly contaminated post-consumer waste while yielding higher amounts of monomers and oligomers with high purity. The rate of hydrolysis depends on the type of functional group, backbone structure, morphology, degree of crystallinity, porosity of the polymer as well as the operating temperature and pH.

Hydrolysis can be further categorized as neutral, acidic, and alkaline hydrolysis. Hydrolysis of polyethylene terephthalate (“PET”), low-density polyethylene (“LDPE”), and polystyrene (“PS”) yields different oligomers and monomers depending on the hydrolysis conditions. PET hydrolysis yields major monomeric compounds of ethylene glycol and terephthalic acid as shown in Structural Formulae (1) and (2). Soluble PET fragments such as mono-(2-hydroxyethyl) terephthalate and bis(2-hydroxyethyl) terephthalate, have been reported. Acidic hydrolysis of PET produces a high yield of terephthalic acid (“TPA”) and affects the purity of ethylene glycol (“EG”) while the neutral hydrolysis of PET affects the purity of TPA. The PET hydrolysis under alkaline conditions reported a higher depolymerization rate (˜95%) under mild conditions. Weight loss of LDPE was studied under alkaline and neutral hydrolysis, and faster degradation and morphology changes were observed under alkaline hydrolysis. LDPE hydrolysis produces alcohol groups (C—OH) by associating a proton and ketone group (C═O) by associating electron. Furthermore, hydroperoxide groups are often evident in LDPE oxidation. Acidic hydrolysis of LDPE yields glutaric acid and succinic acid, and further photo-oxidation leads to liquid byproducts such as carboxylic acid and formic acid. Alkaline hydrolysis of PS produces the styrene monomer by breaking the chain due to its characteristic of the free radical mechanism, and further oxidation of styrene forms substances with carboxyl and carbonyl groups.

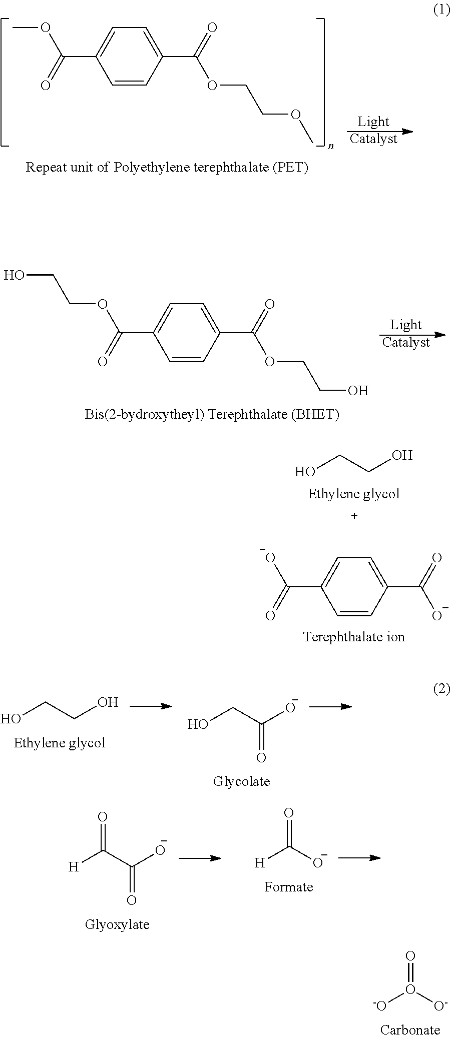

The degradation and hydrolysis of PET forms by-products such as bis(2-hydroxyethyl) terephthalate, ethylene glycol, and terephthalic acid, which are valuable and revenue generation materials as shown in Table 4. Ethylene glycol is an important organic compound and chemical intermediate used in many industrial processes such as energy, plastic, automobiles, and chemicals. The global production and consumption of ethylene glycol are about 20 million metric tons in 2010 with an estimated increase of 5%-10% per year. TPA is one of the most important chemicals that is used in the synthesis of PET and has a global annual production of 50 million tons (in 2014) with an annual growth rate of 5%. With the exhaustion of non-renewable resources, including oil, coal, and natural gas, the production of TPA from organic wastes, especially PET waste, has gained significant attention.

TABLE 4
Polyethylene terephthalate (PET) degradation
byproducts and respective application

| Valuable byproduct | Application |
|---|---|
| Bis(2-hydroxyethyl) | Used to produce materials such as polyester, |
| terephthalate | polyurethane resins, and plasticizer |
| Ethylene glycol | H₂ production |
| | Fuel cell |
| | Chemical production such as glyoxal, glycolic |
| | acid, methyl glycolate, acetals, fructose |
| | Applied as an anti-freezing agent, coolant, |
| | and heat transfer fluid |
| Terephthalic acid | Polyester filaments, yarn, and fiber production |
| | Synthesis of polyester terephthalate |

There is a necessity to standardize and harmonize methods when treating microplastic contaminated water. Conventional water treatment methods including coagulation, flocculation, and filtration were found to be not fully applicable to address this issue. High energy demand, high cost, and toxic byproduct formation are concerns with traditional treatment methods for microplastic removal. Microplastics as solid waste are still environmentally harmful even with the high-efficient removal from water. Therefore, removal and efficient degradation of microplastics are emerging concerns with a much greener approach. Hence, what is needed is to: develop high-performance photocatalysts that utilize visible light; degrade microplastics in water and quantify the degradability; and photo-reform and produce hydrogen utilizing microplastics in the substrate. The compositions and methods of the present invention improve photo-reforming system efficiency for H₂ production through photocatalysis, polymer hydrolysis, and chemical reactions.

### BRIEF SUMMARY OF THE INVENTION

Embodiments of the present invention relate to a composition for producing hydrogen comprising: a semiconductor; and a plurality of metal atoms attached to a surface of the semiconductor, wherein each of the plurality of metal atoms is not in contact with another metal atom attached to the surface of the semiconductor. In another embodiment, the plurality of metal atoms comprises a platinum group metal. In another embodiment, the platinum group metal comprises platinum. In another embodiment, the plurality of metal atoms comprises a precious metal. In another embodiment, the precious metal comprises gold. In another embodiment, the plurality of metal atoms comprises zinc. In another embodiment, the semiconductor comprises a metal oxide. In another embodiment, the metal oxide comprises titanium dioxide. In another embodiment, the semiconductor comprises silicon dioxide.

Embodiments of the present invention also relate to a method for producing hydrogen, the method comprising: pretreating a polymer, wherein pretreating the polymer comprises contacting the polymer with a first aqueous solution; contacting the polymer with a second aqueous solution; contacting the polymer with a nanocomposite, the nanocomposite comprising: a semiconductor; and a metal attached to a surface of the semiconductor; contacting the nanocomposite with radiation; oxidizing the polymer; and forming hydrogen. In another embodiment, the method further comprises forming disodium terephthalate. In another embodiment, the method further comprises forming terephthalic acid. In another embodiment, the polymer comprises polyethylene terephthalate. In another embodiment, the polymer comprises low-density polyethylene. In another embodiment, the polymer comprises polystyrene. In another embodiment, pretreating the polymer comprises contacting the polymer with a base. In another embodiment, pretreating the polymer comprises contacting the polymer with ethanol. In another embodiment, pretreating the polymer comprises contacting the polymer with an acid. In another embodiment, the method further comprises contacting a metal with a semiconductor in the absence of light to form a nanocomposite. In another embodiment, the method further comprises adjusting the pH of the second aqueous solution.

Further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. In the drawings:

FIG. 1 is a flow diagram showing the photo-reforming hydrogen production system, according to an embodiment of the present invention;

FIG. 2 is a flow diagram showing the polyethylene terephthalate pretreatment process, according to an embodiment of the present invention;

FIG. 3 is a flow diagram showing plastic pretreatment and photo-reforming process for H₂ production, according to an embodiment of the present invention;

FIG. 4 is a diagram showing a comparison of the photo-reforming process with the photo-oxidation process, according to an embodiment of the present invention;

FIG. 5 is a graph showing the hydrogen yield of single Pt atom deposited TiO₂ with precursor, according to an embodiment of the present invention;

FIGS. 6A and 6B are graphs showing H₂ production and production rate using Au/TiO₂, respectively, according to an embodiment of the present invention;

FIGS. 7A and 7B are graphs showing H₂ production under ethanol (“EtOH”)-induced pretreatment and potassium hydroxide (“KOH”) and sulfuric acid (“H₂SO₄”) pretreatment conditions, respectively, according to an embodiment of the present invention;

FIG. 8 is a graph showing H₂ production using PET, LDPE, and PS as the polymer sacrificial agents with Au/TiO₂ as the catalyst at different loading ratios, according to an embodiment of the present invention;

FIG. 9 is a graph showing different pretreatment conditions on H₂ production using PET as the polymer and Pt/TiO₂ as the catalyst, according to an embodiment of the present invention;

FIG. 10 is a graph showing a comparison of H₂ production with EtOH-induced pretreatment and extracted byproducts, according to an embodiment of the present invention;

FIGS. 11A and 11B are graphs showing attenuated total reflectance-Fourier transform infrared spectroscopy (“ATR-FTIR”) of extracted TPA and Na₂TP, respectively, according to an embodiment of the present invention;

FIGS. 12A, 12B, and 12C are graphs showing ATR-FTIR scans of residual PET, LDPE, and PS, respectively, according to an embodiment of the present invention;

FIG. 13 is a graph showing mass loss of PET before and after pretreatment, according to an embodiment of the present invention;

FIG. 14 is a graph showing derivative mass loss of PET, according to an embodiment of the present invention;

FIG. 15 is a graph showing derivative mass loss of PET within the temperature range of 550° C. to 700° C., according to an embodiment of the present invention;

FIG. 16 is a graph showing weight reduction of PET at 400° C., according to an embodiment of the present invention;

FIG. 17 is a graph showing weight reduction of PET at 450° C., according to an embodiment of the present invention;

FIG. 18 is a graph showing weight reduction of PET at 700° C., according to an embodiment of the present invention;

FIGS. 19A, 19B, 19C, 19D, and 19E are images and graphs showing transmission electron microscope (“TEM”) images of 0.7% Pt/TiO₂ at a 10 nm scale, a TEM image of Pt/TiO₂ at a 2 nm scale, a TEM image of Au/TiO₂ at a 20 nm scale, a Pt/TiO₂ surface plot around line absorbance, and a Pt/TiO₂ surface profile of line absorbance, respectively, according to an embodiment of the present invention;

FIGS. 20A, 20B, 20C, and 20D are graphs showing bandgap energy analyses of Au/TiO₂, Pt/TiO₂, and Zn/TiO₂, according to an embodiment of the present invention;

FIGS. 21A, 21B, 21C, and 21D are graphs showing full wavelength X-ray photoelectron spectroscopy (“XPS”) scans of catalysts, variation of Pt 4f XPS scan with different Pt doping contents, variation of Ti2p, and variation of O1s of pure TiO₂, Au/TiO₂, and Pt/TiO₂, respectively, according to an embodiment of the present invention;

FIGS. 22A and 22B are graphs showing the effect of different catalysts on the hydrogen yield, and hydrogen yield at different Pt loading rates, respectively, according to an embodiment of the present invention;

FIGS. 23A, 23B, and 23C are graphs showing variation of hydrogen production with pretreatment conditions for KOH at 25° C. with Pt/TiO₂, Au/TiO₂, and Zn/TiO₂ catalysts, KOH at 40° C. with Pt/TiO₂, Au/TiO₂, and Zn/TiO₂ catalysts, and EtOH-induced pretreatment of PET with Pt/TiO₂ and Au/TiO₂ catalysts, respectively, according to an embodiment of the present invention;

FIGS. 24A and 24B are graphs showing hydrogen production at different pH conditions in the reactor with PET treated according to an EtOH-induced method, and PET pretreatment with KOH according to a mixed method using Au/TiO₂ and Pt/TiO₂ catalysts, respectively, according to an embodiment of the present invention;

FIG. 25 is a graph showing the variation of I/I0 of Rhodamine B solution degraded using pure TiO₂, Au/TiO₂, Pt/TiO₂, and Pt/ZnO, according to an embodiment of the present invention;

FIGS. 26A-26B are photographs showing TEM images of TiO₂ and Au/TiO₂ at 300 k magnification, according to an embodiment of the present invention;

FIG. 27 is a graph showing the Fourier transform infrared spectroscopy (“FTIR”) spectrum of TiO₂ and Au doped TiO₂, with new bands occurring due to Au doping, according to an embodiment of the present invention;

FIG. 28 is a graph showing the variation of I/I0 of Rhodamine B solution with UV exposed time using Au/TiO₂ catalyst, according to an embodiment of the present invention;

FIG. 29 is a graph showing the decrease of UV absorbance at 554 nm of Rhodamine B over time using TiO₂ and Au/TiO₂ as catalysts, according to an embodiment of the present invention;

FIG. 30 is a graph showing the comparison of FTIR spectra of PET during the photo-reforming process over time, according to an embodiment of the present invention;

FIG. 31 is a graph showing the comparison of PET degradation with Au/TiO₂ under UV and visible light irradiation, according to an embodiment of the present invention;

FIG. 32 is a graph showing the headspace gas composition analysis with different time intervals, according to an embodiment of the present invention;

FIG. 33 is a graph showing the quantitative analysis of H₂ production using Au/TiO₂ and TiO₂ as the photocatalyst over time, according to an embodiment of the present invention, according to an embodiment of the present invention;

FIG. 34 is a graph showing the quantitative analysis of H₂ production using Au/TiO₂ as catalysts, according to an embodiment of the present invention;

FIG. 35 is a graph showing the quantitative analysis of the H₂ production rate using Au/TiO₂ as catalysts, according to an embodiment of the present invention;

FIG. 36 is a graph and a table showing the UV high pressure lamp (60 mW/cm²)-hydrogen yield (μmolg_{cat}⁻¹) for various plastics over time, according to an embodiment of the present invention;

FIG. 37 is a graph and a table showing the UV high pressure lamp (60 mW/cm²)-hydrogen production rate (μmolg_{cat}⁻¹ h⁻¹) over time, according to an embodiment of the present invention;

FIG. 38 is a graph and a table showing visible light (45 mW/cm²)-hydrogen yield (μmolg_{cat}⁻¹)) for various plastics over time, according to an embodiment of the present invention; and

FIG. 39 is a graph and a table showing the visible light (45 mW/cm²)-hydrogen production rate μmolg_{cat}⁻¹ h⁻¹) for various plastics over time, according to an embodiment of the present invention.

### DETAILED DESCRIPTION OF THE INVENTION

Embodiments of the present invention relate to a composition for producing hydrogen and/or polymer degradation. The composition may comprise a semiconductor, and a metal, wherein single metal atoms or metal clusters are attached to the surface of the semiconductor. The metal may include, but is not limited to, platinum, zinc, gold, or a combination thereof. The single metal atoms and/or plurality of metal atoms may not be in contact with another metal atom and/or plurality of metal atoms.

Embodiments of the present invention also relate to a method of hydrogen production, the method comprising: a semiconductor; and a plurality of metal atoms attached to a surface of the semiconductor, wherein each of the plurality of metal atoms is not in contact with another metal atom attached to the surface of the semiconductor. The plurality of metal atoms may comprise a platinum group metal, a noble metal, a precious metal, a base metal or a combination thereof. The metal may comprise platinum, gold, zinc, or a combination thereof. In another embodiment, the platinum group metal comprises platinum. The semiconductor may comprise a metal oxide, a silicon oxide, silicon, germanium, nitrides, sulfides or a combination thereof. The semiconductor may comprise titanium oxide.

Embodiments of the present invention also relate to method for making a nanocomposite, the method comprising: heating a semiconductor nanoparticle; contacting the semiconductor with a first alcohol; contacting the semiconductor with a metal in the absence of light to attach the metal to the semiconductor; and drying the semiconductor. The metal may further comprise contacting the metal attached to the semiconductor with a second alcohol. The first alcohol may comprise methanol. The second alcohol may comprise ethanol. The metal may comprise chloroplatinic acid (“H₂PtCl₆”). The metal may be attached to the semiconductor as an individual metal atom or plurality of metal atoms.

Embodiments of the present invention also relate to method for pretreating a polymer, the method comprising: contacting a polymer with a reagent to form a polymer and reagent solution; adjusting the temperature of the polymer; and adjusting the pH of the polymer and reagent solution. The reagent may comprise a base. The base may comprise potassium chloride, sodium chloride, ethanol, sulfuric acid, or a combination thereof. The adjusting the temperature may comprise increasing the temperature. Adjusting the pH of the polymer and reagent solution may comprise adjusting the pH to a neutral pH or an acidic pH.

Embodiments of the present invention relate to a method of hydrogen production, the method comprising: contacting a polymer with a nanocomposite; and contacting the polymer with radiation. The method may further comprise pretreating the polymer. The method may further comprise degrading and/or photo-reforming the polymer.

The method may use polymer waste as a resource to generate hydrogen by using a photo-reforming process. The method may be driven by high wavelength energy from solar and/or UV energy and nanocomposites used as the catalyst to increase the efficiency of the process. The production of H₂ and degradation of a polymer may yield oxidizing and/or other chemical conditions that may disinfect and/or remove biological and/or organic matter from the environment.

The composition and method may be used for multiple purposes, including but not limited to, treatment of hydrocarbon spills; removal of pathogens from plasma, agricultural products, and/or medical-grade reagents; removal of polymers in blood; removal of biological matter from sewage for wastewater treatment; removal of polymers from an aqueous solution, including, but not limited to, salt water, fresh water, grey water, sea water, produced water, brines, drinking water, or a combination thereof.

The terms “nanocomposite”, “nanocomposite catalyst”, “catalyst”, and “photocatalyst” are used interchangeably in the specification and drawings.

The terms “degrade” and “photo-reform” are used interchangeably in the specification and drawings and are defined to mean to change and/or break down the carbon-hydrogen bonds, carbon-oxygen bonds, and/or hydrogen-oxygen bonds of a polymer.

The terms “metal” or “metals” are defined herein as a compound, mixture, or substance comprising a metal atom. The term “metal” or “metals” includes, but is not limited to, metal hydroxides, metal oxides, metal salts, elemental metals, metal ions, non-ionic metals, minerals, or a combination thereof.

The terms “polymer” or “polymers” as used herein means a substance that has a molecular structure comprising a plurality of similar units bonded together. The polymer may comprise, but is not limited to acrylic polyamide, polyester, high- and low-density polyethylene (“HD/LD-PE”), polyethylene terephthalate (“PET”), polypropylene (“PP”), polystyrene (“PS”), and polyvinyl chloride (“PVC”), terephthalic acid (“TPA”), ethylene glycol (“EG”), or a combination thereof.

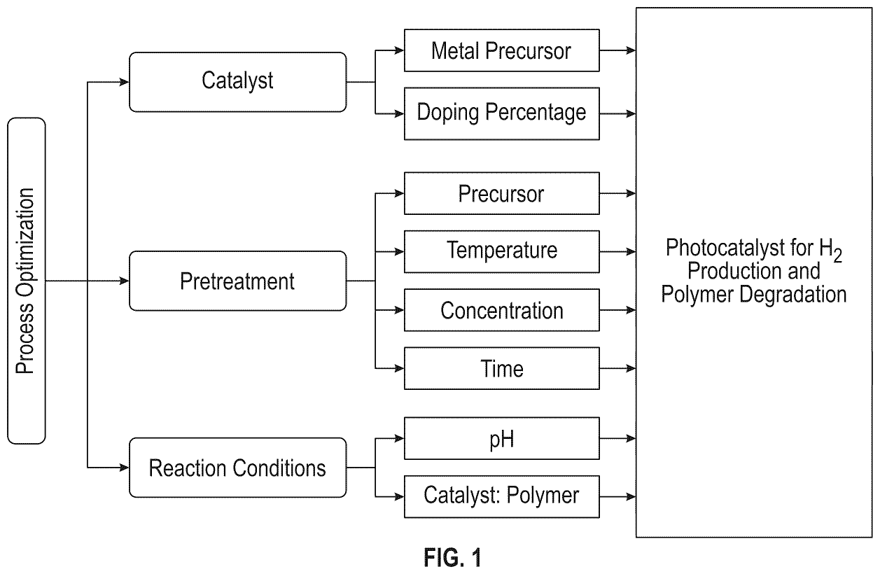

Turning now to the figures, FIG. 1 shows the photo-reforming hydrogen production system. The photo-reforming hydrogen production system requires the selection of a catalyst, polymer pretreatment conditions, and reaction conditions. The catalyst constituents comprise metal precursor and a metal doping percentage. The pretreatment conditions require the selection of a precursor, a temperature, a concentration, and a time period for pretreatment. The reaction conditions require the selection of a pH and polymer type.

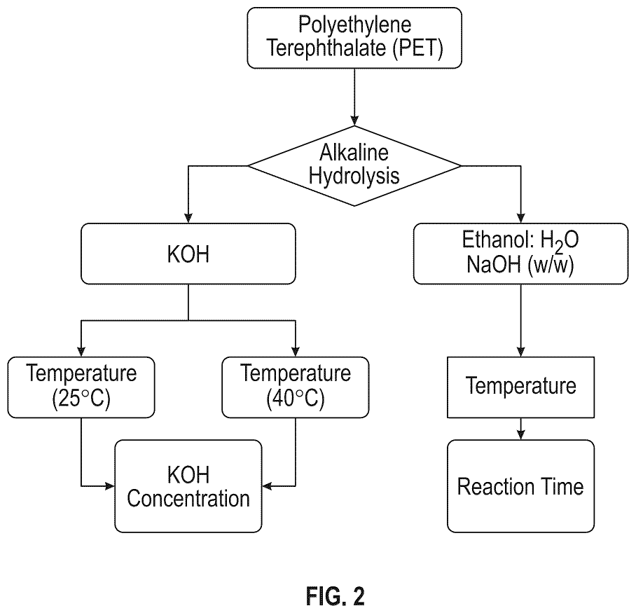

FIG. 2 shows the polyethylene terephthalate pretreatment process. Polyethylene terephthalate undergoes alkaline hydrolysis by either contact with KOH or ethanol with sodium hydroxide (“NaOH”). Alkaline hydrolysis using KOH may occur at 25° C. or 40° C. at a known KOH concentration. Alkaline hydrolysis using ethanol and NaOH occurs at a known temperature for a defined reaction time.

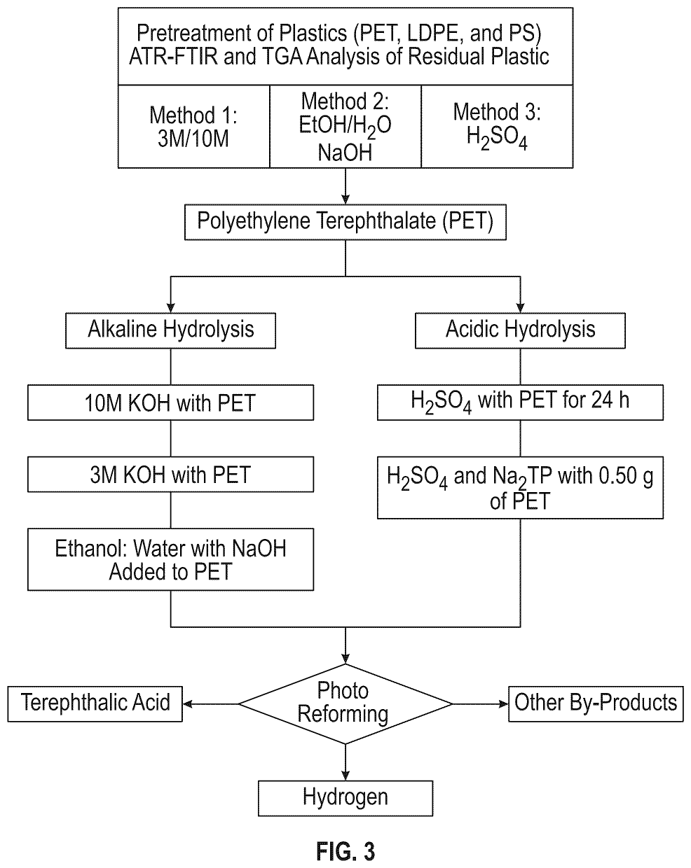

FIG. 3 shows plastic pretreatment and photo-reforming process for H₂ production. Common plastic materials PET, LDPE, and PS are selected as sacrificial agents. Three main pretreatment processes applied to hydrolyze the plastics before photo-reforming process are shown in FIG. 3. During the photo-reforming process, the added plastic amount is adjusted to optimize H₂ production.

PET hydrolysis occurs under two major processes: alkaline and acidic pretreatment. PET may act as a sacrificial agent under the pretreatment conditions shown in FIG. 3. An additional alkaline hydrolysis method is applied using a 10 M KOH solution. Acidic hydrolysis is catalyzed by adding Na₂TP to the solution as show in FIG. 3. All these pretreatments are applied into 0.5 g of finely cut polymer at 25° C. PET, LDPE, and PS were pretreated using three different methods shown in FIG. 3.

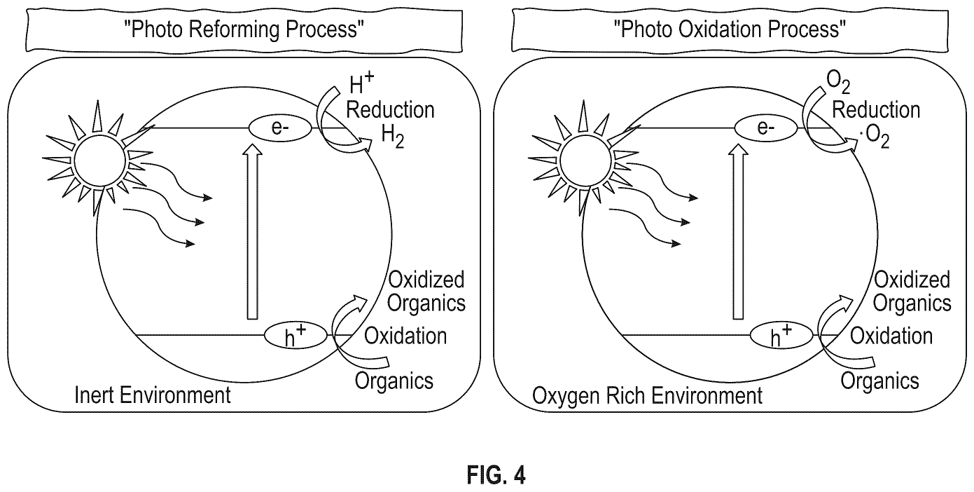

FIG. 4 shows a comparison of the photo-reforming process with the photo-oxidation process. In an inert environment, organics, e.g., polymers, are oxidized to produce a free electron that reduces available hydrogen ions to produce hydrogen gas. In an oxygen-rich environment, organics, e.g., polymers, are oxidized to produce a free electron that reduces oxygen gas to oxygen comprising a free radical.

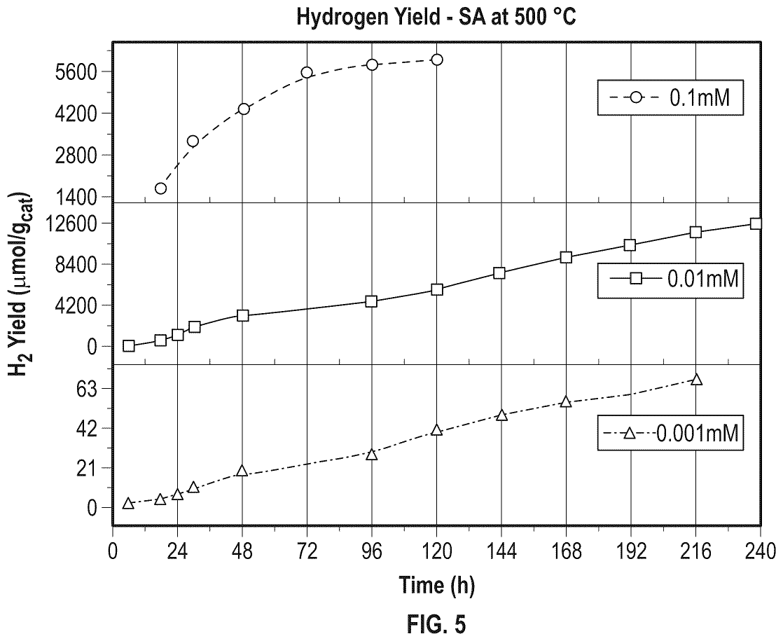

FIG. 5 shows hydrogen yield of single Pt atom deposited TiO₂ with precursor (chloroplatinic acid (H₂PtCₗ₆·6H₂O)) concentrations of 0.1 mM, 0.01 mM, and 0.001 mM under dark deposition method. Comparison among different metal-doped TiO₂ composites shows that single-atom Pt-doped TiO₂ results in the highest hydrogen yield. Doping percentages may be at least about 0.3%, about 0.3% to about 6.0%, about 0.7% to about 5.0%, about 1% to about 4%, about 1.5% to about 3.5%, or about 6.0% Pt to form Pt/TiO₂. Hydrogen production ability is increased when increasing the number of Pt single atoms on TiO₂. When increasing the doping percentage from 0.7% to 1.5%, the H₂ production increased by 146%. Pt doping percentage and H₂ production may not be linearly correlated because the higher charge recombination rate due to high defect availability in the catalyst with high Pt dosage may reduce the efficiency of H₂ production. Without being bound to a particular theory, the enhancement of photocatalytic activity may be related to the creation of electronic defects coupled to the rutile phase of TiO₂ when lower Pt percentages doped in the TiO₂. Metalized Pt may be present in the catalyst nanoparticles when the Pt percentage is increased, thereby improving the catalytic activity by enhancing the charge separation and lowering the recombination of charge carriers, electrons, and holes. Pt loading less than 0.7% (w/w) may be a single Pt atom deposited to form Pt/TiO₂.

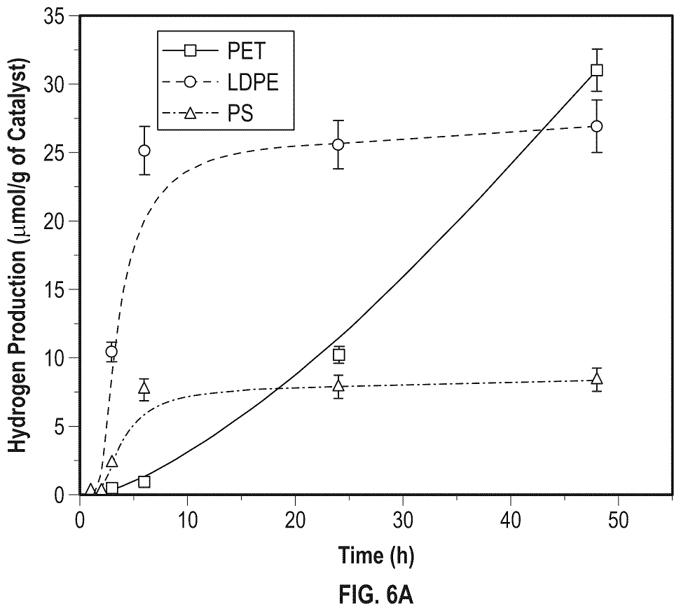

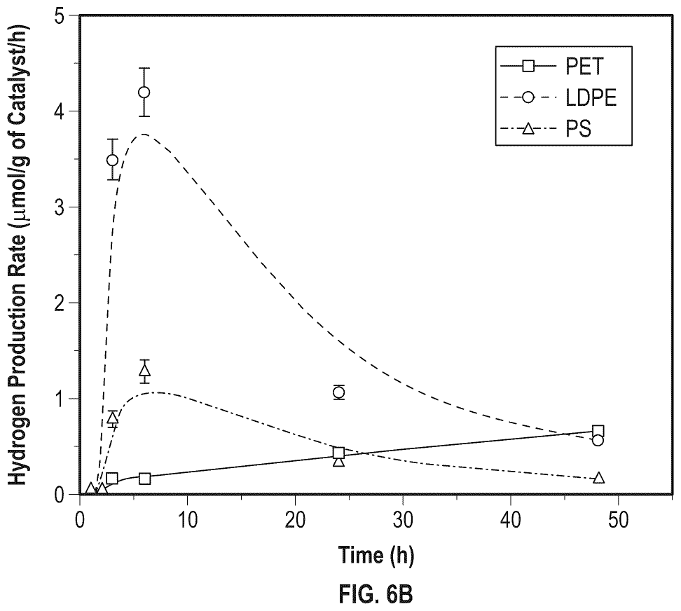

FIGS. 6A and 6B show H₂ production and production rate using Au/TiO₂, respectively, with different polymers under UV light (60 mW/cm²). The H₂ production and production rate using PET, LDPE, and PS as sacrificial agents after pretreatment with 3 M KOH for 24 hours are shown. The highest rate of H₂ production of 0.65 μmol of H₂ per gram of catalyst per hour (“μmol/g_{cat}/h”) is observed using PET as the sacrificial agent at 48 hours. LDPE shows a 0.56 μmol/g_{cat}/h H₂ production rate, 13% lower than PET, while PS as the sacrificial agent shows the lowest H₂ production rate of 0.17 μmol/g_{cat}/h. The H₂ production rate increases with time using PET as the sacrificial agent, whereas the H₂ production rate decreases using LDPE and PS in the reaction mixture. A maximum H₂ yield of 31.04 μmol of H₂ per gram of catalyst (“μmol/g_{cat}”) is observed after 48 hours during photo-reforming with PET, while 26.89 μmol/g_{cat} and 8.38 μmol/g_{cat} of H₂ yield are obtained after 48 hours in the reactors with LDPE and PS, respectively. The H₂ production reaches a maximum at 6 hours and remains constant in both the reactors with LDPE and PS, while PET shows continued rapid increase in H₂ production even at 48 hours.

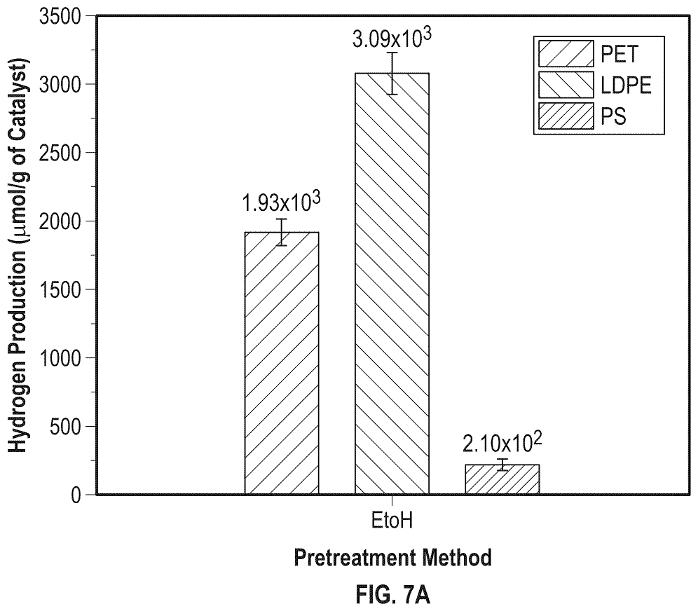

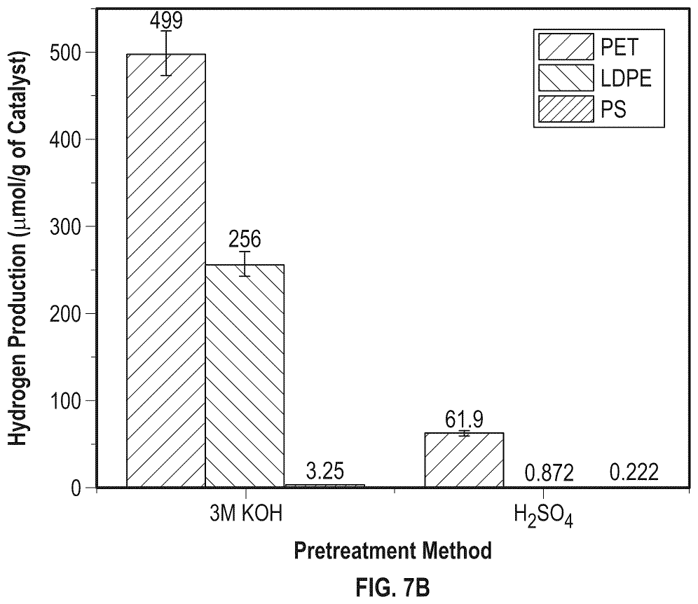

FIGS. 7A and 7B show cumulative H₂ production under EtOH-induced pretreatment and 3 M KOH and 1 M H₂SO₄ pretreatment conditions, respectively after 48 hours. FIG. 7A shows that EtOH pretreatment of LDPE yielded the highest hydrogen production. FIG. 7B shows that pretreatment of PET with KOH and H₂SO₄ yielded the highest hydrogen production. The highest H₂ production is observed when LDPE is used with the EtOH-induced pretreatment method, which is 3,091 μmol/g_{cat}. It is 160% of the H₂ produced with PET under the same pretreatment conditions. PS has the lowest H₂ production under EtOH-induced pretreatment, which is 210 μmol/g_{cat}. EtOH-induced pretreatment generated the highest amount of H₂ considering all the plastic (PET, LDPE, and PS) sacrificed reactions. H₂SO₄ pretreated polymers show the lowest H₂ evolution after 48 hours. The reaction solutions with LDPE and PS produce 0.87 μmol/g_{cat} and 0.22 μmol/g_{cat} of H₂, and PET applied reactor measures 61 μmol/g_{cat} H₂ production. FIGS. 7A and 7B show the dependency of H₂ production on the sacrificial agent as well as the applied pretreatment conditions.

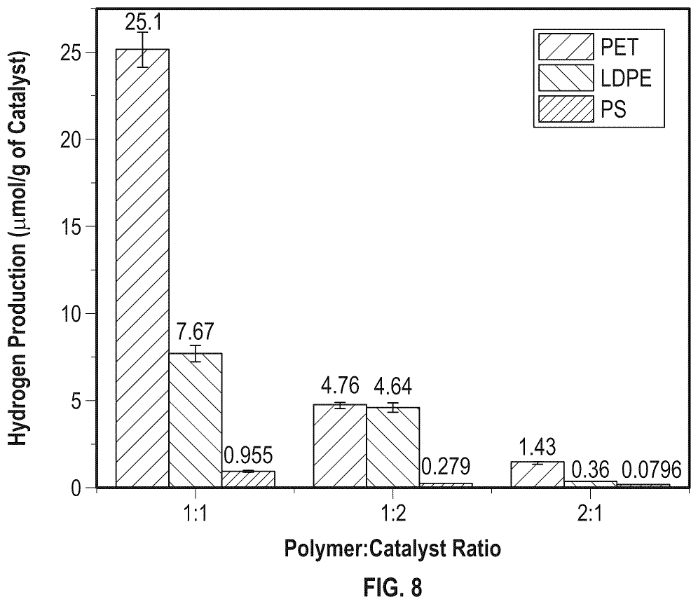

FIG. 8 shows H₂ production using PET, LDPE, and PS as the polymer sacrificial agents with Au/TiO₂ as the catalyst at different loading ratios under UV irradiation (60 mW/cm²). A weight of 0.25 g, 0.50 g, and 1.00 g of different polymers is used with 0.50 g of catalyst to optimize for H₂ production. Here, the polymer-to-catalyst ratio is stated as 1:2, 1:1, and 2:1 for the reactors with 0.25 g, 0.50 g, and 1.00 g of polymer, respectively. Polymer-to-catalyst of 1:1 shows the highest H₂ production for all three polymers. PET with a polymer-to-catalyst ratio of 1:1 shows the highest H₂ production of 25.14 μmol/g_{cat} among all the polymers under all loading conditions. LDPE at the loading ratio of 1:1 indicates the second-highest H₂ production of 7.67 μmol/g_{cat}, which is 30% of the produced H₂ using PET as a sacrificial agent. Even though the highest H₂ production of PS was observed under the reactor condition of 1:1, it is as low as 0.96 μmol/g_{cat} compared to the H₂ production from other polymers under the same condition. The least efficient H₂ production was measured at the polymer-to-catalyst ratio of 2:1 for all considered polymers. H₂ production under 2:1 loading conditions was 5.6%, 4.6%, and 8.2% of produced H₂ at 1:1 loading conditions for PET, LDPE, and PS, respectively. The H₂ yield for all polymers in the 1:2 loading condition is higher than those in the 2:1 loading condition. PET, LDPE, and PS applied reactors produce 4.75 μmol/g_{cat}, 4.64 μmol/g_{cat}, and 0.28 μmol/g_{cat} of H₂, respectively, under 1:2 loading conditions which are 18.9%, 60.4%, and 28.4% of produced H₂ in 1:1 loading condition.

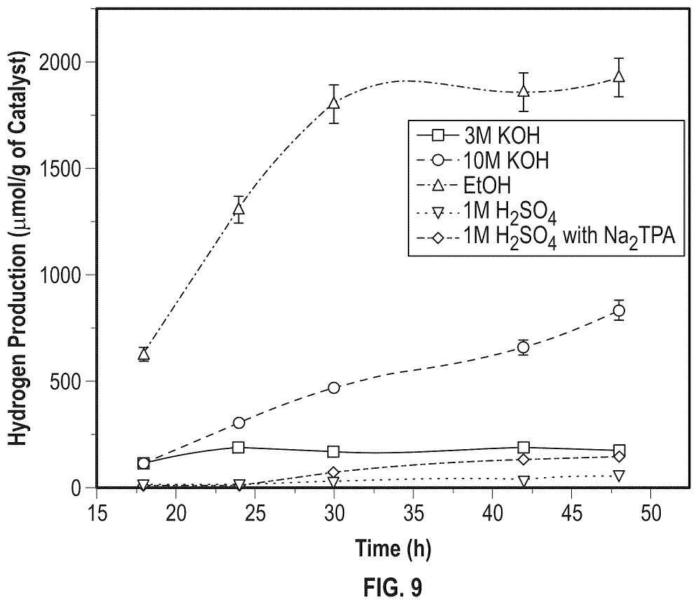

FIG. 9 shows the effect of different pretreatment conditions on H₂ production using PET as the polymer and Pt/TiO₂ as the catalyst with a 1:1 polymer-to-catalyst ratio. Pretreatment of polymer is identified as a factor affecting the H₂ production efficiency under the photo-reforming process. FIG. 9 shows the variation of H₂ production under different pretreatment conditions using PET as the sacrificial agent. Pt/TiO₂ is the catalyst after pretreating 0.50 g of PET.

The pretreatment of PET and its impact on H₂ production is shown in FIG. 9. The highest H₂ production is observed in a reactor with PET after EtOH-induced pretreatment. Cumulative H₂ production after 48 hour is 1,925 μmol/g_{cat} under the EtOH-induced pretreatment method, which is 2.3 times higher than that of PET pretreated with 10 M KOH (832 μmol/g_{cat} at 48 hour). Without being bound by a particular theory, higher H₂ production under EtOH-induced pretreatment method may be due to the complete hydrolysis of PET. Alkaline hydrolysis of PET with 3 M and 10 M KOH pretreatment conditions positively affect H₂ production compared to acidic pretreatment conditions. Acidic pretreatment of PET leads to the lowest H₂ production using 1 M H₂SO₄. As a result, the H₂ production at 48 hours is 61.8 μmol/g_{cat}, which is about 8% of the H₂ yield after applying alkaline hydrolysis of PET with 10 M KOH. Acidic hydrolysis with H₂SO₄ is catalyzed with Na₂TP, a byproduct formed during hydrolysis. Increased H₂ production from 61 μmol/g_{cat} to 148 μmol/g_{cat} is observed with PET pretreated with Na₂TP catalyzed acidic hydrolysis with 1 M H₂SO₄, which is about the H₂ production under alkaline 3 M KOH pretreatment. The 10 M KOH pretreated PET produced about five times the quantity of H₂ at 48 hour compared to the H₂ production after 3 M KOH pretreated PET. This indicates that the increased alkaline concentration during the pretreatment process positively affects the H₂ production.

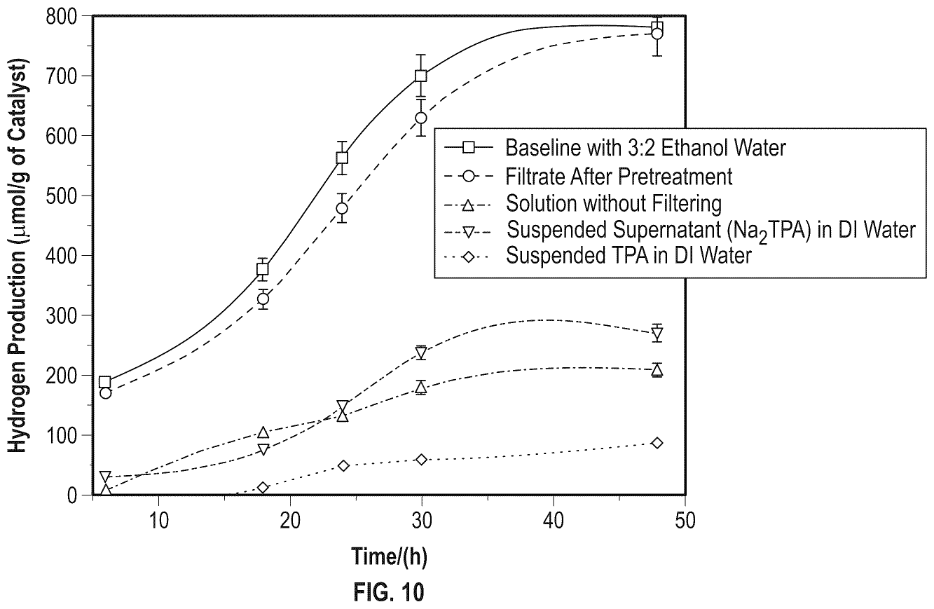

FIG. 10 shows a comparison of H₂ production with EtOH-induced pretreatment and extracted byproducts. The EtOH-induced pretreatment led to significantly higher hydrolysis of PET and also higher efficiency of H₂ production compared to other pretreatment methods. H₂ production is further analyzed and evaluated using extracted filtrate, supernatant, and derived byproducts. After the pretreatment process, Au/TiO₂ is added as the catalyst, and H₂ production is evaluated for 48 hours. The maximum H₂ production is observed using the filtrate after the pretreatment as the reaction mixture. This mixture may comprise of ethylene glycol, water, residual EtOH, and other soluble byproducts with the catalyst. H₂ production under this condition is as high as using 60% (v/v) EtOH as the baseline, indicating that the resulting byproduct formed after PET hydrolysis contributed to H₂ production showing similar kinetics to the H₂ production from EtOH. This byproduct may be ethylene glycol, a major monomer created by PET hydrolysis. The H₂ production decreased by about 73% when the photo-reforming experiment is performed without filtering the pretreated solution due to lower photo absorbance efficiency caused by residual particles. The hydrolysis of PET creates a white precipitate which is filtered and used in photo-reforming experiments after suspending in deionized water with Au/TiO₂ as the catalyst. Photo-reforming of this suspension results in an H₂ yield of 270 μmol/g_{cat} which is 35% of H₂ produced from the filtrate. This precipitate may be Na₂TP and residual PET. Further photo-reforming with the catalyst produces H₂. Precipitated Na₂TP and residues may be soluble in water. Adding H₂SO₄, may form a white precipitate of TPA. The suspended solution of TPA may be photo-reformed, and headspace gas analyzed for H₂. The TPA suspension generates 87.2 μmol/g_{cat} of H₂, indicating the possibility of further oxidation of TPA.

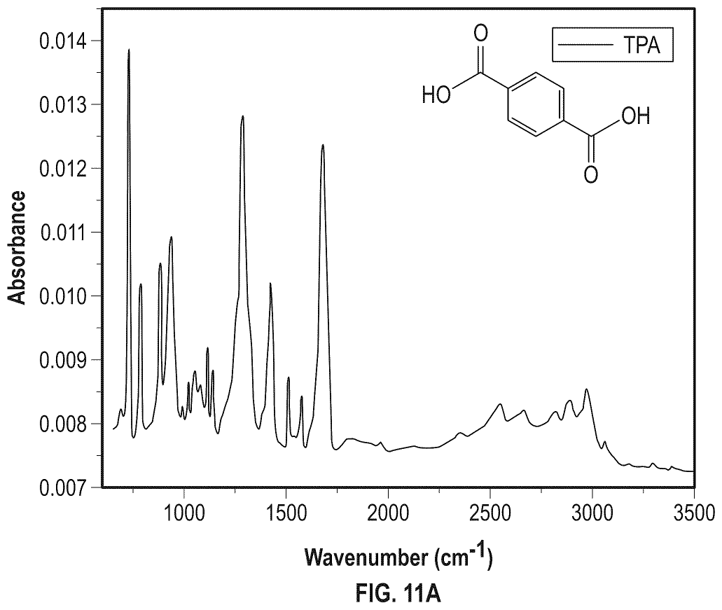

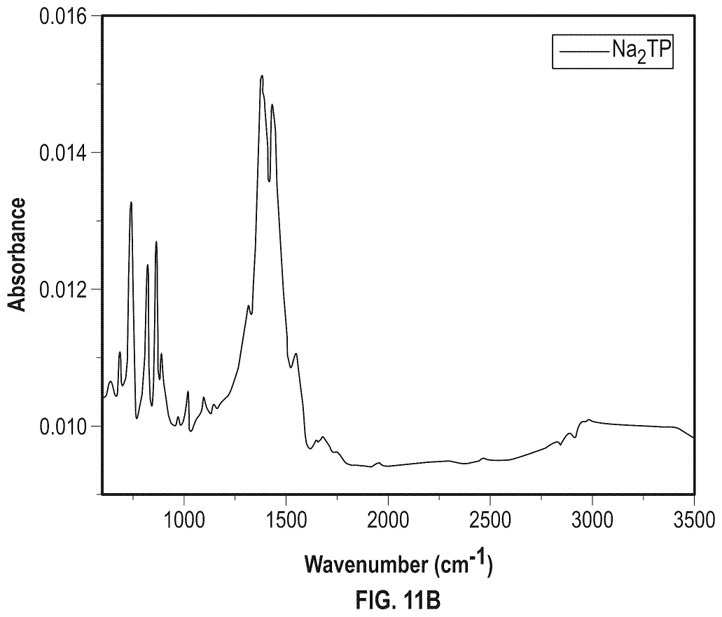

FIGS. 11A and 11B show ATR-FTIR of extracted TPA and Na₂TP, respectively. The extracted Na₂TP and TPA are analyzed using ATR-FTIR, and functional groups were verified. TPA contains two carbonyl groups, one on each benzene ring. The stretching vibrations of these carbonyl groups typically appear as strong peaks around 1700 cm⁻¹-1720 cm⁻¹. The C═C stretching vibrations of the aromatic rings appear as peaks around 1615 cm⁻¹. The stretching vibrations of the carboxylic acid functional group in TPA typically appear as a strong, broad peak in the range of 2500 cm⁻¹-3000 cm⁻¹. This peak is associated with the stretching vibrations of the O—H bond and may be influenced by hydrogen bonding. The presence of carboxylic acid groups in terephthalic acid enables hydrogen bonding interactions. The broad peak at 940 cm⁻¹ corresponds to O—H bending vibrations of the carboxyl (“COOH”) group in the terephthalic acid. The disappearance of the O—H bend bond from the TPA is observed in Na₂TP, which results from the sodium insertion into the molecule to form the disodium terephthalate. The absorption bands at 1394 cm⁻¹ and 1566 cm⁻¹ are attributed to the C═C stretching vibration from the aromatic structure.

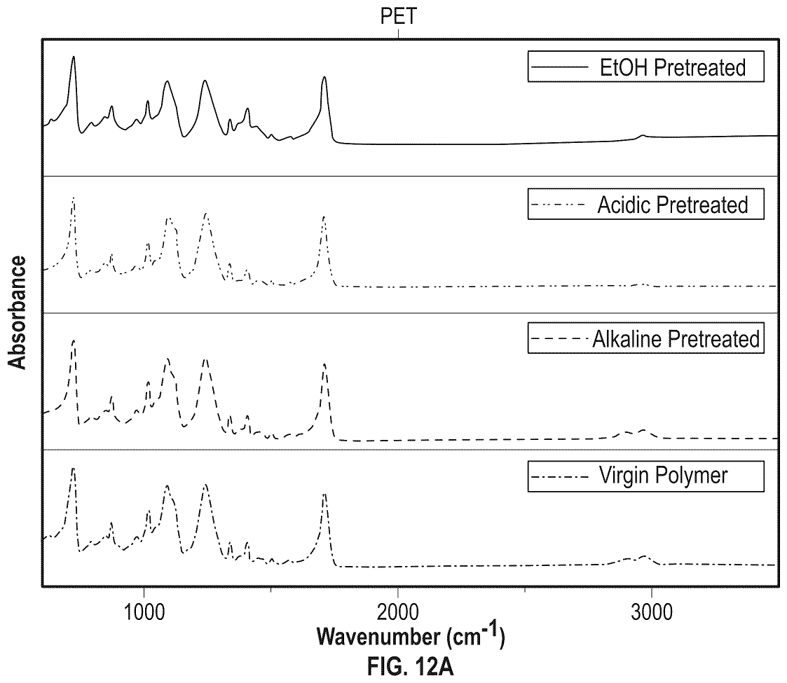

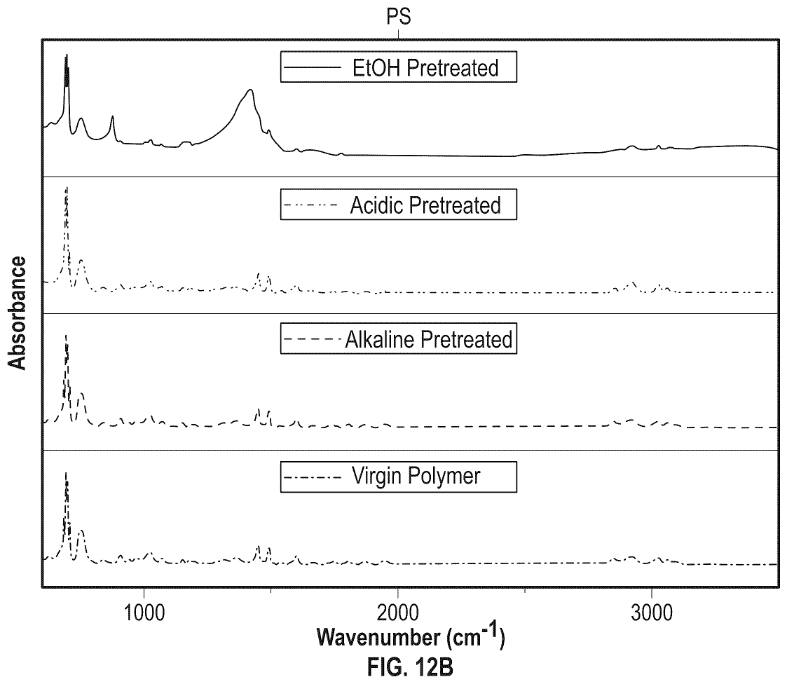

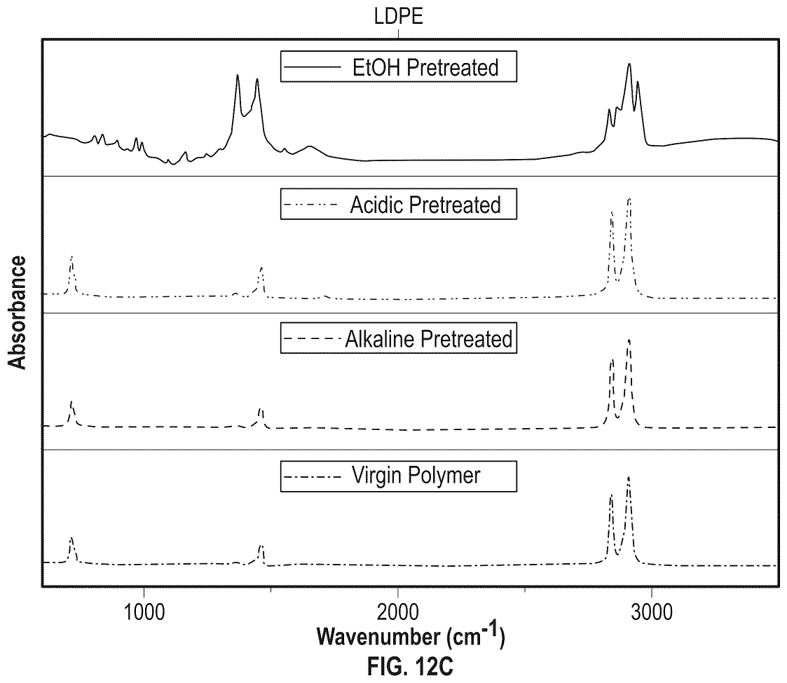

FIGS. 12A, 12B, and 12C show a comparison of ATR-FTIR scans of residual PET, LDPE, and PS, respectively, before and after pretreatment. FIGS. 12A, 12B, and 12C present the ATR-FTIR spectrum of 3 M KOH pretreated polymers for 24 hours. Functional groups do not indicate a significant difference when compared to virgin polymer particles under the alkaline pretreatment with KOH and acidic pretreatment conditions. However, EtOH-induced pretreatment shows a considerable difference in the FTIR spectra compared to virgin polymers. Hydrolysis of PET under EtOH-induced pretreatment may cause ester (“COO—”) interchange reactions resulting in the formation of new ester linkages or modification of existing ester groups in PET. The ester linkages or modification of the existing ester group in PET may appear as the broadened peak at 1730 cm⁻¹. The alcohol group of EtOH may replace the ester groups in the PET surface by breaking down the ester linkages and forming ether (“—O—”) groups resulting in a widened peak around 1500 cm⁻¹. The potential of introducing alkyl groups onto the PET surface by EtOH may cause a broadened peak around 1470 cm⁻¹. The appearance of a broad peak around 3300 cm-1-3500 cm⁻¹ may indicate the presence of hydroxyl stretching vibrations due to the formation of hydroxyl groups on the LDPE surface. The formation of carbonyl groups, such as ketones or aldehydes, may be observed as a peak around 1720 cm⁻¹, representing the stretching vibrations of carbonyl bonds in the spectra after EtOH-induced pretreatment of LDPE. Additionally, the esterification reaction promoted by EtOH may contribute to the broadened peak around 1730 cm⁻¹-1750 cm⁻¹ due to the presence of ester groups. A difference of peaks around 1375 cm⁻¹-1450 cm⁻¹ may indicate the presence of alkyl groups introduced from EtOH to the LDPE surface after hydrolysis. The formation of carbonyl groups, ester linkages, and ether groups replacing the hydrogen atoms in the PS backbone may cause the observed peak broadening at around 1450 cm⁻¹. The surface changes observed in all polymers under EtOH-induced pretreatment may affect the surface properties, such as wettability and adhesion characteristics.

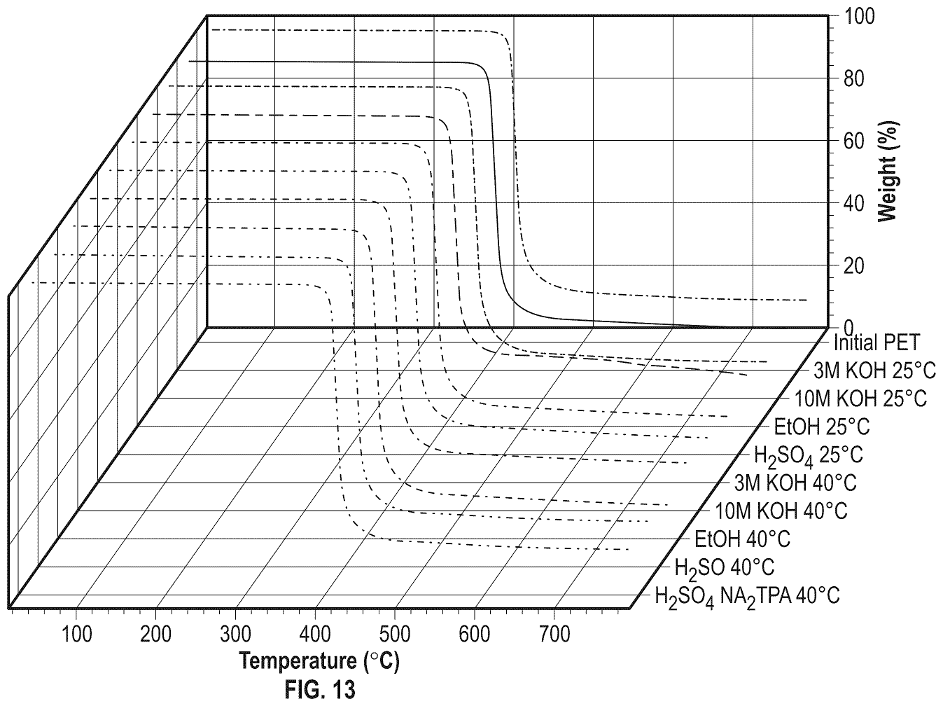

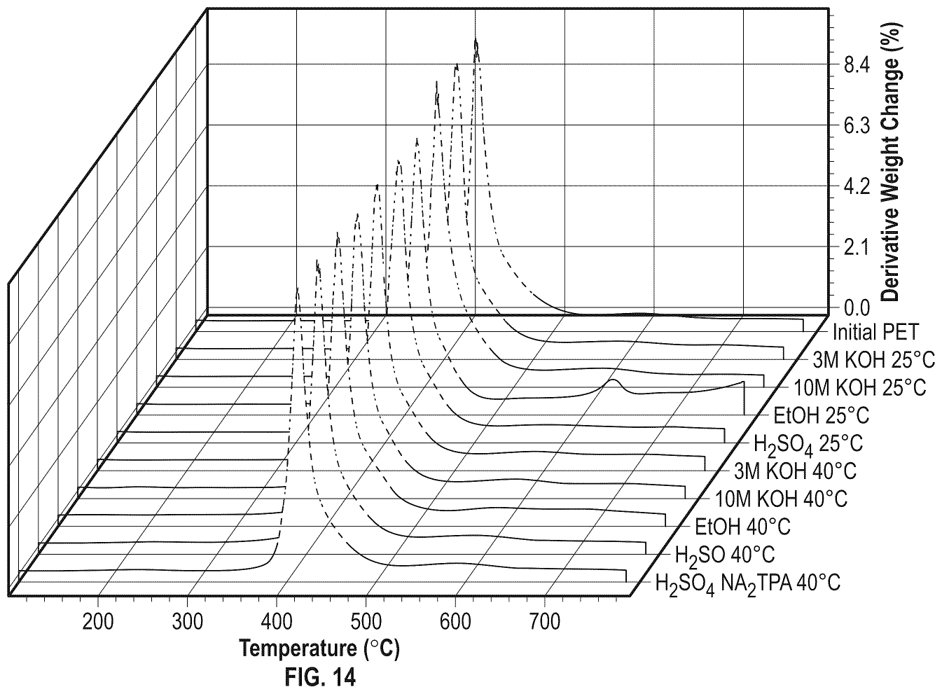

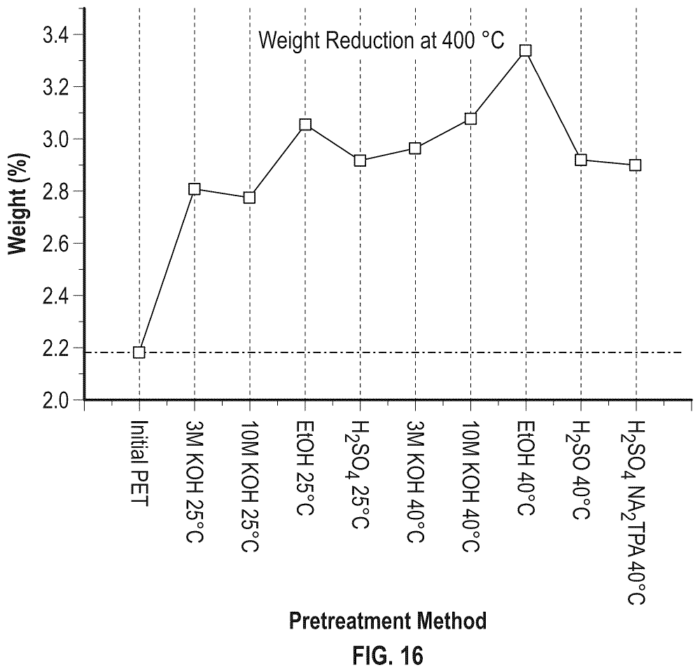

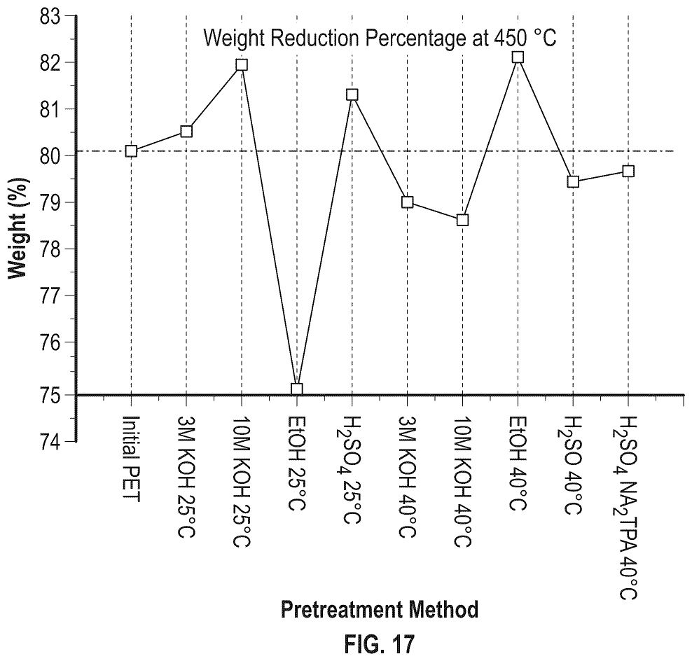

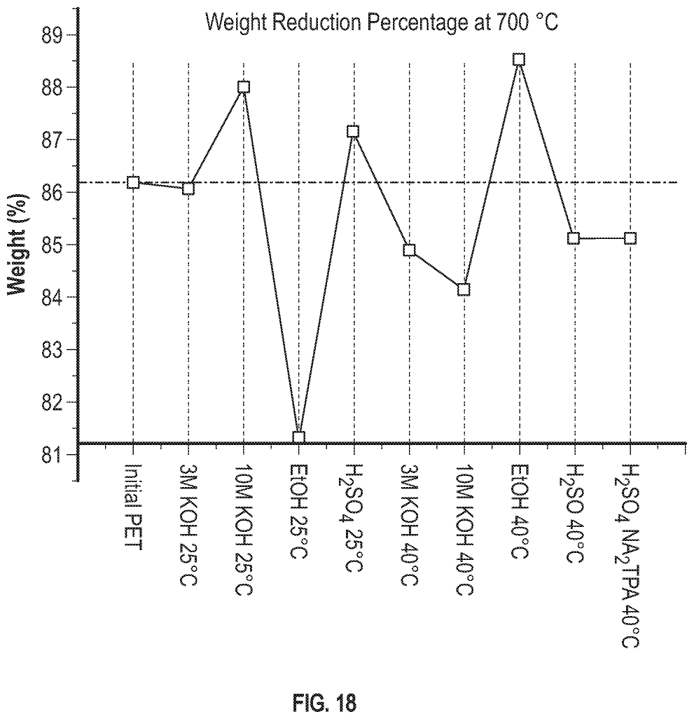

FIGS. 13, 14, 15, 16, 17, and 18 show the thermogravimetric analysis (“TGA”) for residual plastic. Specifically, FIGS. 13, 14, 15, 16, 17, and 18 show a comparison of mass loss of PET before and after pretreatment; derivative mass loss of PET; derivative mass loss of PET within the temperature range of 550° C. to 700° C.; weight reduction of PET at 400° C.; weight reduction of PET at 450° C.; and weight reduction of PET at 700° C., respectively. The thermogravimetric curves of virgin PET show four stages of thermal degradation corresponding to the thermal degradation of different fragments of compounds in the PET polymer structure. The weight loss at the first stage may be due to volatiles such as CO and CO₂. During the second stage, C₂H₆O₂ and C₂H₄O may be degraded. Loss of C₄O₂ occurs in the third stage, and RCO—OR compounds are degraded at the final stage. FIGS. 13 and 14 show the mass loss and derivative mass loss of PET after pretreatment and compared with the virgin PET sample. The TGA curves show a single-stage decomposition briefly around the onset temperature of 400° C.

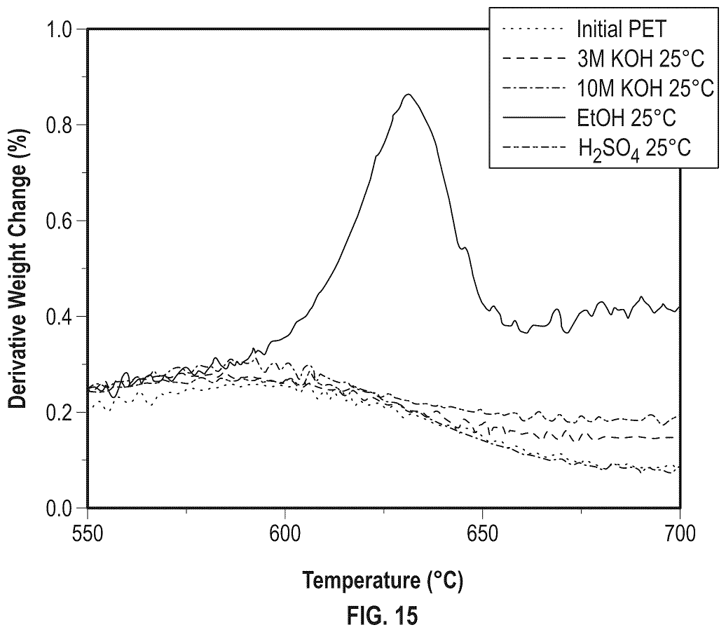

FIGS. 15, 16, 17, and 18 show mass loss differences can be observed at different temperatures due to different pretreatment conditions. Derivative weight change of PET after EtOH-induced pretreatment shows a weight change of around 6.7% at 615° C. whereas virgin PET sample shows a slight weight reduction of 1.65% at 615° C. Without being limited to a particular theory, this may be because EtOH-induced pretreatment increases the decomposition ability of the PET. When comparing the mass loss at 400° C. after the first and second decomposition of PET, all the pretreated PETs show higher weight loss compared to the virgin PET sample due to a higher rate of decomposition and loss of volatiles due to higher temperature. The highest mass loss of 3.1% is observed in the EtOH-induced pretreated PET, and mass loss increases to 3.3% when increasing the pretreatment temperature from 25° C. to 40° C. under the EtOH-induced pretreatment method. All the other pretreated PET samples show increased mass loss with increasing temperature. Mass reduction at temperatures 450° C. and 700° C. shows a similar trend corresponding to different pretreatment options. Most of the pretreated PET samples measure lower mass loss compared to virgin PET samples due to higher oxidation potential or higher decomposition potential. TGA analysis shows that hydrolysis of PET positively influences the decomposition potential.

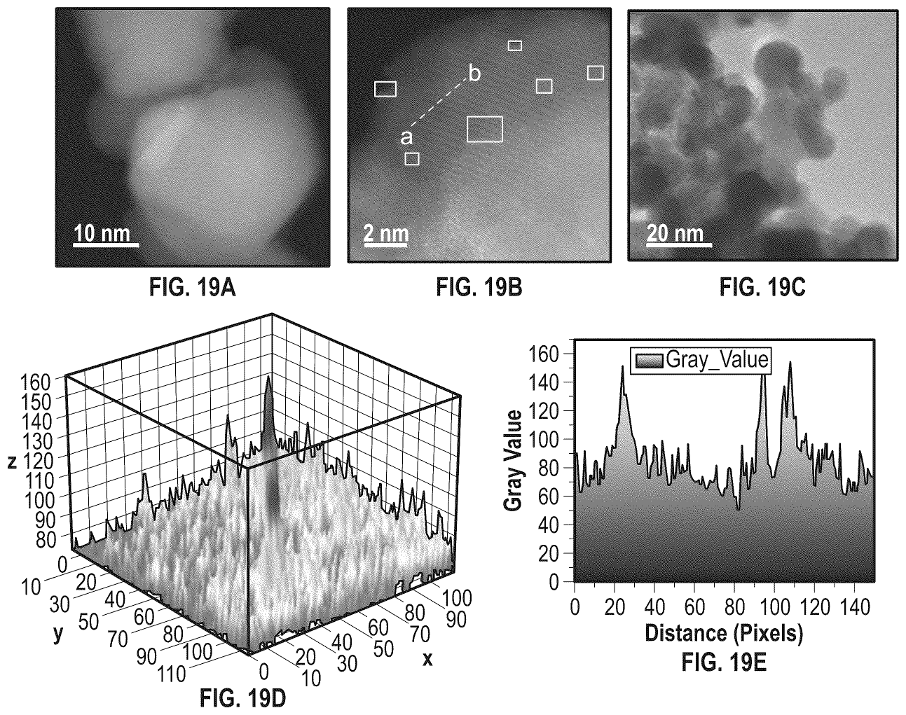

FIGS. 19A, 19B, 19C, 19D, and 19E show a TEM image of 0.7% Pt/TiO₂ at a 10 nm scale, a TEM image of Pt/TiO₂ at a 2 nm scale, a TEM image of Au/TiO₂ at a 20 nm scale, a Pt/TiO₂ surface plot around line absorbance, and a Pt/TiO₂ surface profile of line absorbance, respectively. The particle size of the TiO₂ semiconductor is in the range of 15 nm to 25 nm as shown in FIG. 19C. Pt particle size is in the range of 0.38 nm to 1 nm. Au particle size is in the range of 1 nm to 3.5 nm. Particle size distribution and morphology of the pristine TiO₂ are compared against the metal-doped TiO₂ by analyzing the scanning transmission electron microscopy (“STEM”) images. FIG. 19A shows a set of regular lattice fringes of 0.38 nm consistent with the (101) crystal plane of the TiO₂. The TEM particle size analysis shows that the average size of metal composite TiO₂ particles remains the same at 20 nm, indicating that surface modification does not affect the particle size significantly. Random distribution of Pt single atoms on the TiO₂ surface is observed in FIGS. 19A and 19B. The 3D surface plot, as shown in FIG. 19D, and surface profile of line absorbance, as shown in FIG. 19E, indicate the surface deposition and anchoring of Pt single atoms. The presence of single atoms is shown to increase the photocatalytic H₂ production by inhibiting the carrier recombination. The average Au particle deposited on the TiO₂ surface is 2.1 nm, and the random distribution of Au particles on the TiO₂ surface is shown in FIG. 19C.

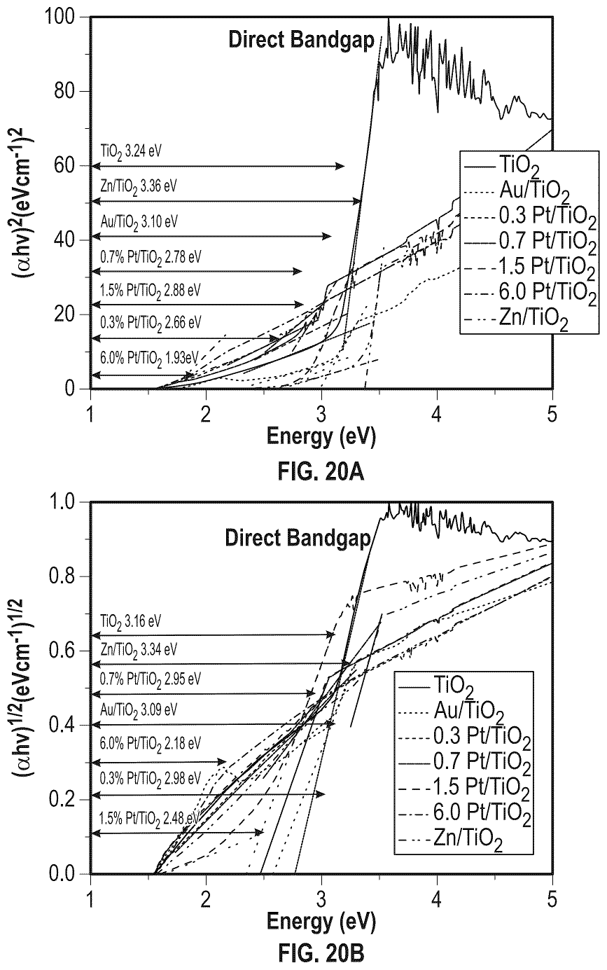

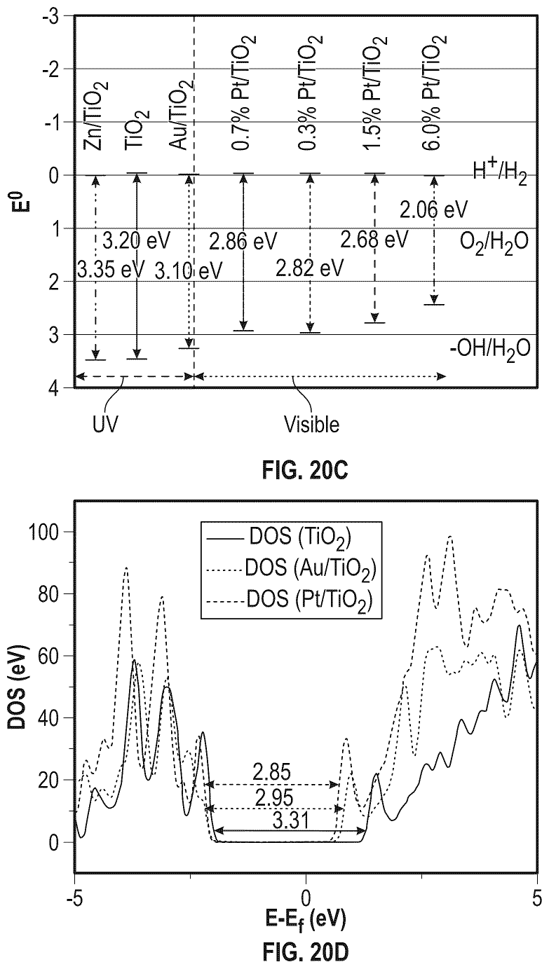

FIGS. 20A, 20B, 20C, and 20D show bandgap energy analyses of Au/TiO₂, Pt/TiO₂, and Zn/TiO₂ considering direct electron transition; indirect electron transition; summary of calculated bandgaps different catalysts; and summary of calculated bandgaps different catalysts with computation simulation of density of state (“DOS”) plot, respectively. The bandgap energy is a parameter for deciding the suitable wavelength range of the light source to power the photo-reforming process. The bandgap energy is calculated using the Tauc plot method using the normalized UV-Vis spectrum of the catalysts dispersed in deionized water, with the assumption of direct and indirect electron transition, as shown in FIGS. 20A and 20B. Bandgap values derived under the assumption of direct electron transition may be higher compared to the values when considering the indirect electron transition. TiO₂ shows a higher bandgap value of 3.20 eV. The bandgap energy is reduced by 0.10 eV when TiO₂ is doped with 5% Au, making the bandgap of Au/TiO₂ to be 3.09 eV to 3.10 eV, as shown in FIG. 20C. The derived bandgap energy range shows that Au/TiO₂ is photoactive in wavelengths greater than 400 nm, near UV to the visible light range of the solar spectrum.

Similar to Au/TiO₂, when doping Pt to the TiO₂ matrix, a significant shrinking in the bandgap is observed, as shown in FIG. 20C. With increasing Pt doping percentages, the average bandgap energy of Pt/TiO₂ decreases from 2.86 eV to 2.06 eV as compared to 3.20 eV of Au/TiO₂. Doping Pt on TiO₂ reduces the bandgap energy from 10% to 40%, depending on the Pt concentration. The average bandgap energy of 0.3% and 0.7% Pt/TiO₂ calculated to be about 2.8 eV and may be due to the smaller Pt dosages as shown in FIG. 20C. The photo-reforming process may occur under visible light with a wavelength range of 400 nm to 500 nm due to the considerable reduction of bandgap energy in Pt/TiO₂.

Zn/TiO₂ shows a bandgap increase of 0.15 eV compared to TiO₂. Without being limited to a particular theory, the bandgap increase may be due to the different synthesis processes of Zn/TiO₂. Under this condition, Zn/TiO₂ behaves similarly to pristine TiO₂. Evaluation of the bandgap of anatase TiO₂, Au/TiO₂, and Pt/TiO₂, confirms the bandgap reduction due to the presence of Au and Pt in the TiO₂ matrix as shown in FIG. 20D.

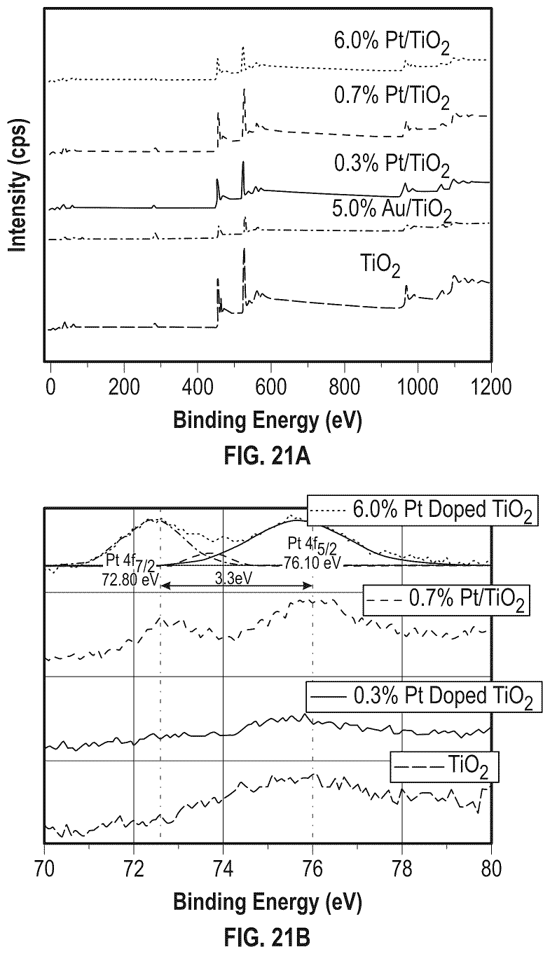

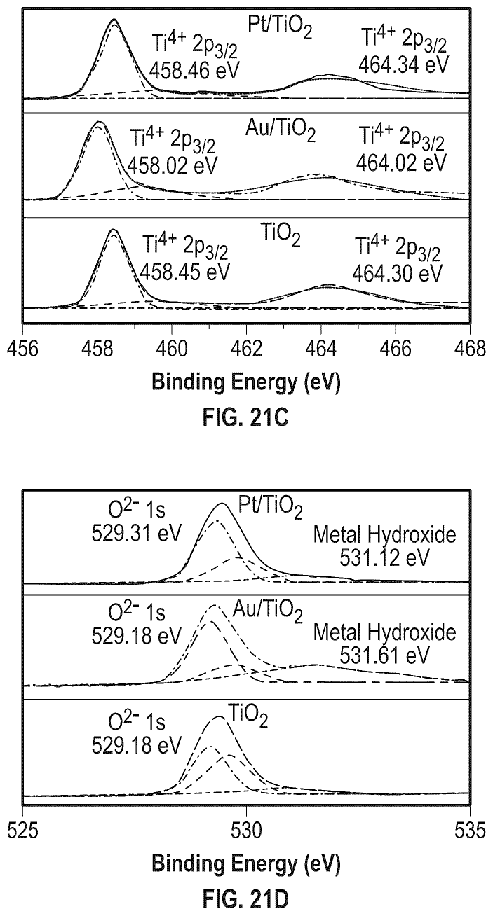

FIGS. 21A, 21B, 21C, and 21D show full wavelength XPS scans of catalysts, variation of Pt 4f XPS scan with different Pt doping contents, variation of Ti2p, and variation of O1s of pure TiO₂, Au/TiO₂, and Pt/TiO₂, respectively. FIG. 21C shows the peak deconvolution of Ti⁴⁺ species. The peak at 458.4 eV in Pt/TiO₂ occurs due to the presence of Ti 2p3/2 in the P25 TiO₂ spectra. The peak of the Ti 2p3/2 in Au/TiO₂ is at around 458.0 eV. As shown in FIG. 21D, the peak of the O1s species is visible at around 529.0 eV in all spectra, and an additional hydroxide peak is present at around 531.0 eV in Au/TiO₂ and Pt/TiO₂ due to the surface adsorption of water molecules. The theoretical weight percentages are lower than the derived weight percentages analyzed using XPS data. All Pt atoms or ions on the TiO₂ surface may cause a low theoretical weight.

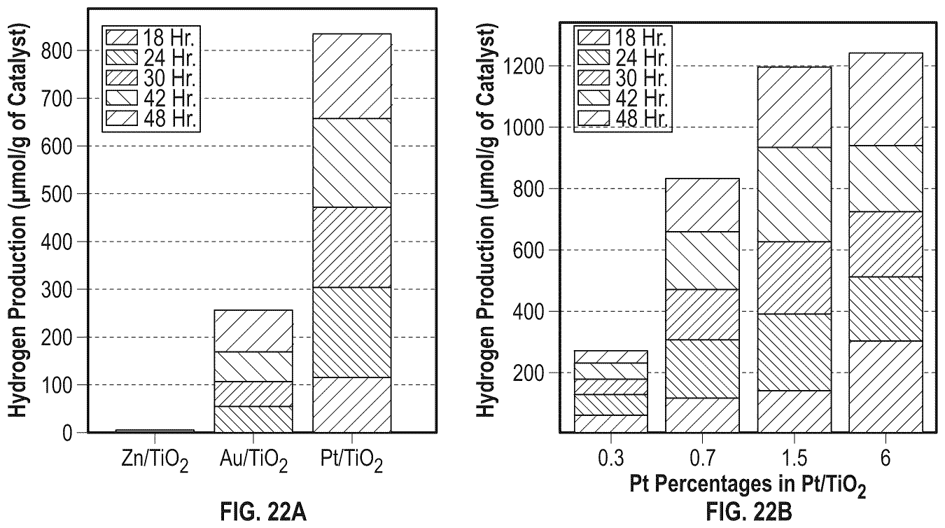

FIGS. 22A and 22B show the effect of different catalysts on the hydrogen yield, and hydrogen yield at different Pt loading rates, respectively, on TiO₂ during 48 hours of PET photo-reforming. Hydrogen production correlates with the respective bandgap energies of catalysts. Among different catalysts, single atom Pt at doping rate of 0.7% Pt/TiO₂ reaches the highest efficiency for hydrogen production, while the photocatalytic efficiency of 5% (w/w) Zn/TiO₂ was lower compared to 0.7% (w/w) Pt/TiO₂. 5% (w/w) Au/TiO₂ is the weakest photocatalyst in hydrogen production. However, the Zn/TiO₂ is not synthesized using P25 Degussa TiO₂ unlike the other catalysts. Without being limited to a particular theory, this may result in lower performance relative to Pt/TiO₂.

Hydrogen production ability is increased when increasing the Pt content on TiO₂, as shown in FIG. 22B. FIG. 22B also shows that the H₂ production increased by 146% when the doping percentage was increased from 0.7% to 1.5%. The Pt doping percentage and H₂ production are not linearly correlated. Without being limited to a particular theory, the correlation is non-linear because the higher charge recombination rate, which may be due to high defect availability in the catalyst with high Pt dosage, reduces the efficiency of H₂ production. Without being limited to a particular theory, the enhancement of photocatalytic activity may be due to electronic defects coupled to the rutile phase of TiO₂ when lower Pt percentages doped in the TiO₂. Metalized Pt may be present in the catalyst nanoparticles when increasing the Pt percentage. The metalized Pt may improve the catalytic activity by enhancing the charge separation and lowering the recombination of charge carriers, electrons, and holes.

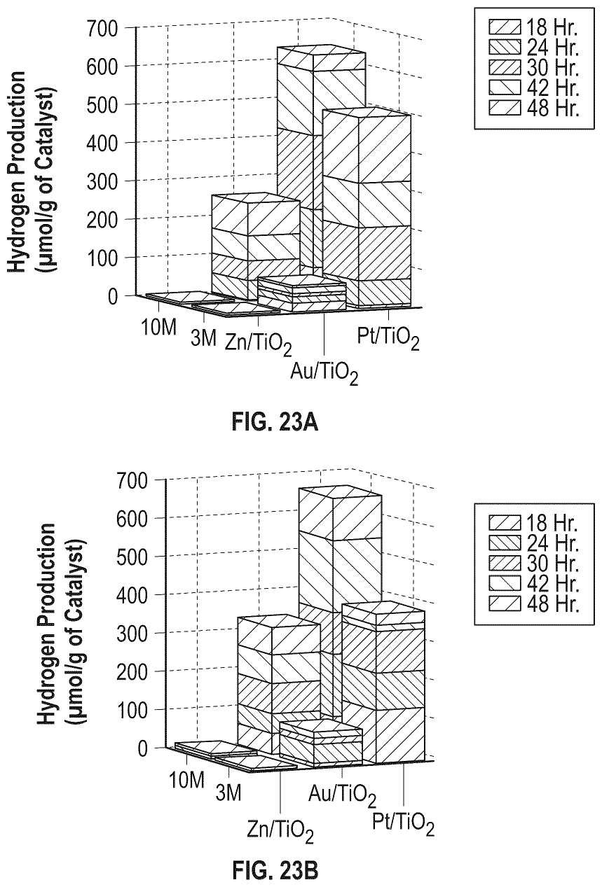

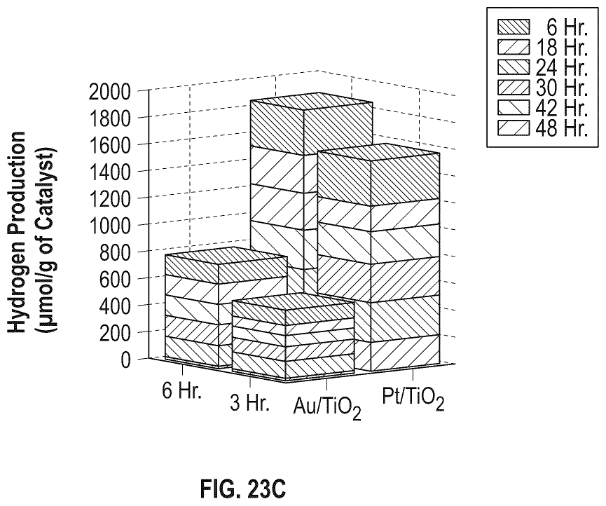

FIGS. 23A, 23B, and 23C show the variation of hydrogen production with pretreatment conditions with 3 M KOH and 10 M at 25° C. with Pt/TiO₂, Au/TiO₂, and Zn/TiO₂ catalysts; 3 M KOH and 10 M KOH at 40° C. with Pt/TiO₂, Au/TiO₂, and Zn/TiO₂ catalysts; and EtOH-induced pre-treatment of PET for 3 hours and 6 hours with Pt/TiO₂ and Au/TiO₂ catalysts, respectively. Hydrogen production increases by 40% using Au/TiO₂ as the catalysts when increasing the pretreatment temperature from 25° C. to 40° C. of the 3 M KOH solutions, shown in FIG. 23B. H₂ yield using Au/TiO₂ and Pt/TiO₂ as catalysts increases by 30% and 4% in 10 M pretreated PET, respectively. An increase in hydrogen production may be observed with increased pretreatment temperature.

A H₂ higher yield is measured using the EtOH-induced pretreatment method compared with the H₂ yield after 10 M KOH pretreatment at 25° C., irrespective of the catalyst. Hydrogen production with EtOH-induced pretreatment for 3 hours is about 2.5 times higher than the 10 M KOH pretreatment, while increasing pretreatment time to 6 hours yields about 3 times higher H₂ production compared to the 10 M KOH pretreatment method with Pt/TiO₂. EtOH-induced pretreatment improves hydrogen yield compared to the 10 M KOH pretreatment method. FIG. 23C shows that the pretreatment method affects the hydrogen yield when using sacrificial agents such as PET which are difficult to hydrolyze. Without being limited by a particular theory, the presence of unreacted EtOH applied in the system during the hydrolysis process of PET may result in an increase an H₂ yield where EtOH is competing as the sacrificial agent with ethylene glycol expected to be formed as a byproduct of PET hydrolysis.

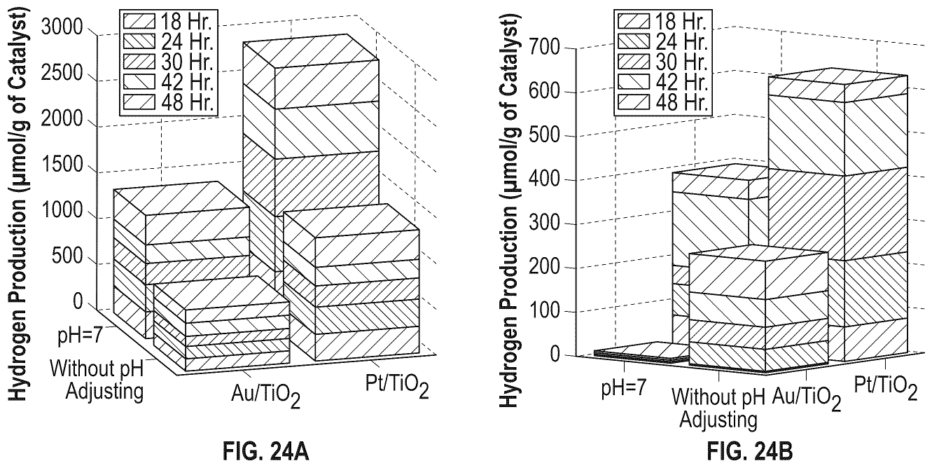

FIGS. 24A and 24B show hydrogen production at different pH conditions in the reactor with PET treated according to an EtOH-induced method, and PET pretreatment with KOH according to a mixed method using Au/TiO₂ and Pt/TiO₂ catalysts, respectively. FIGS. 24A and 24B show H₂ production with and without the pH adjustment in the EtOH-pretreated PET (FIG. 24A) and KOH-pretreated (FIG. 24B) photo-reforming systems. When pH is reduced from >13 to 7, the H₂ yield improves for both catalysts in reactors with EtOH-induced pretreated PET, as shown in FIG. 24A. A 110% increase in H₂ production is observed when using Pt/TiO₂ as the catalyst, and about a 100% increase of H₂ yield is observed in the presence of Au/TiO₂ catalyst when PET was treated with the EtOH-induced pretreatment method. Among the EtOH-induced pretreated systems, the highest H₂ yield of about 2800 μmol/g_{cat} is measured using Pt/TiO₂ as the catalyst at pH 7, as shown in FIG. 24A.

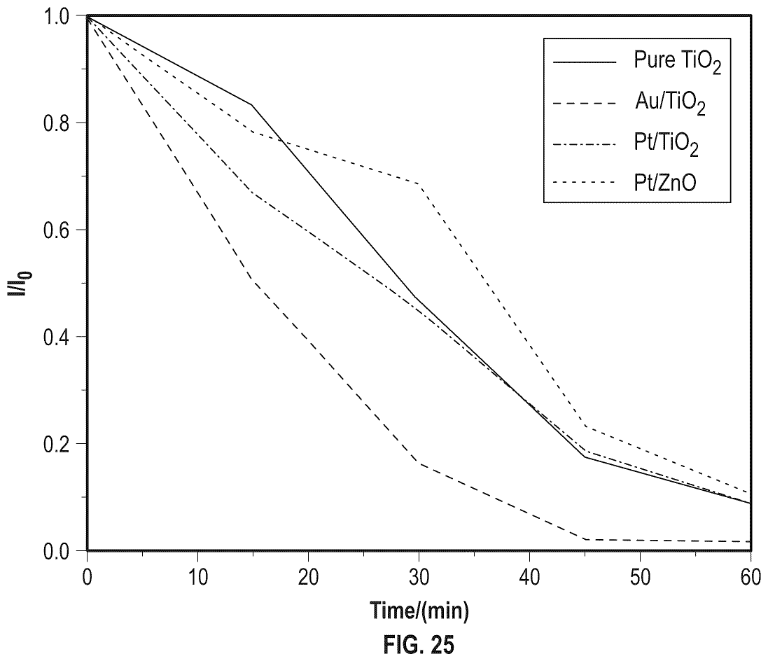

FIG. 25 shows the variation of I/I0 of Rhodamine B solution degraded using pure TiO₂, Au/TiO₂, Pt/TiO₂, and Pt/ZnO. TiO₂, Au/TiO₂, Pt/TiO₂, and Pt/ZnO all result in Rhodamine B degradation over time, with Au/TiO₂ causing the most significant degradation.

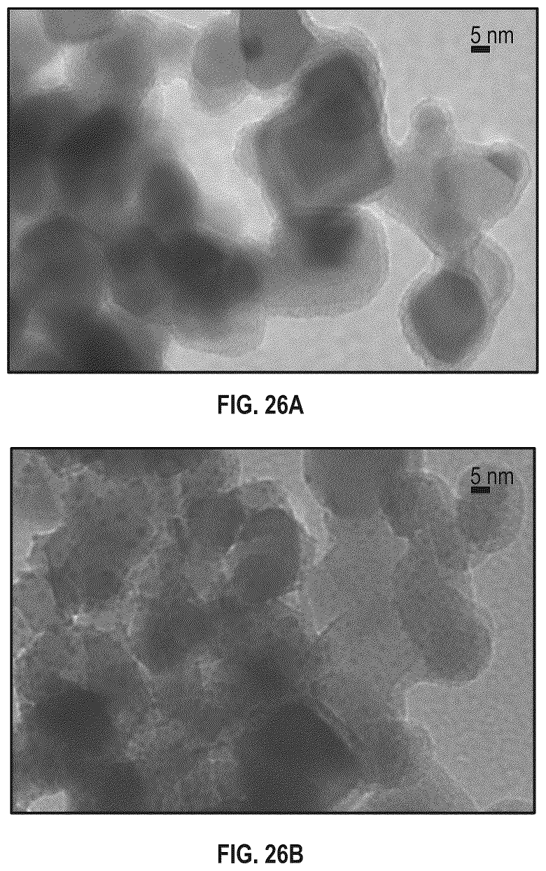

FIGS. 26A-26B show TEM images of TiO₂ and Au/TiO₂ at 300 k magnification. Au is adhered to TiO₂ to form a nanocomposite.

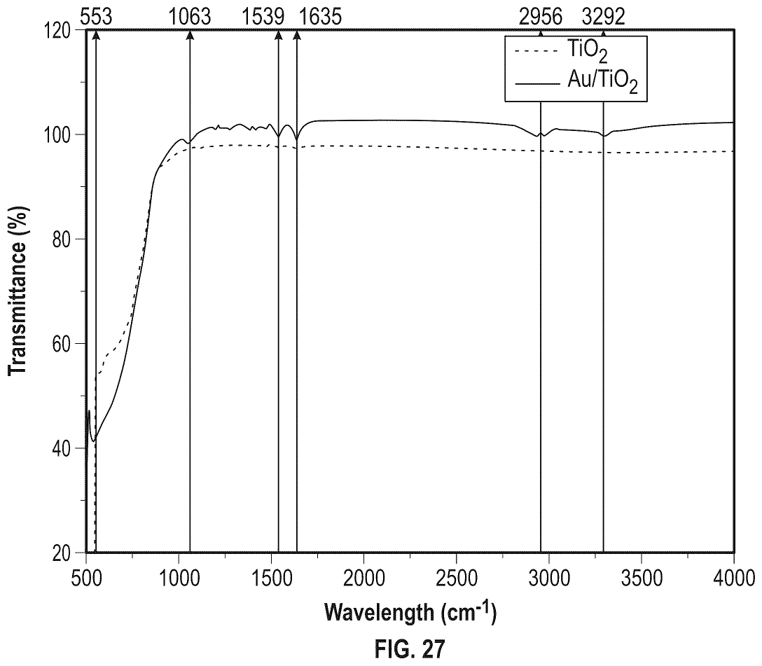

FIG. 27 shows the FTIR spectrum of TiO₂ and Au doped TiO₂, with new bands occurring due to Au doping. The new bands appear at 533, 1063, 1539, 1635, 2956, and 3292 cm⁻¹.

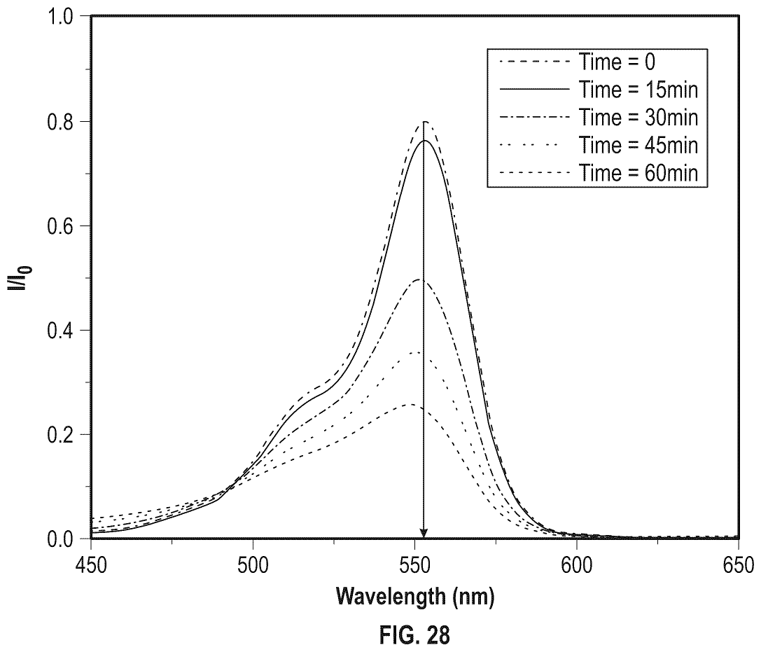

FIG. 28 shows the variation of I/I₀ of Rhodamine B solution with UV exposed time using Au/TiO₂ catalyst, where I0 and I are the initial absorbance and absorbance at the specific time, respectively.

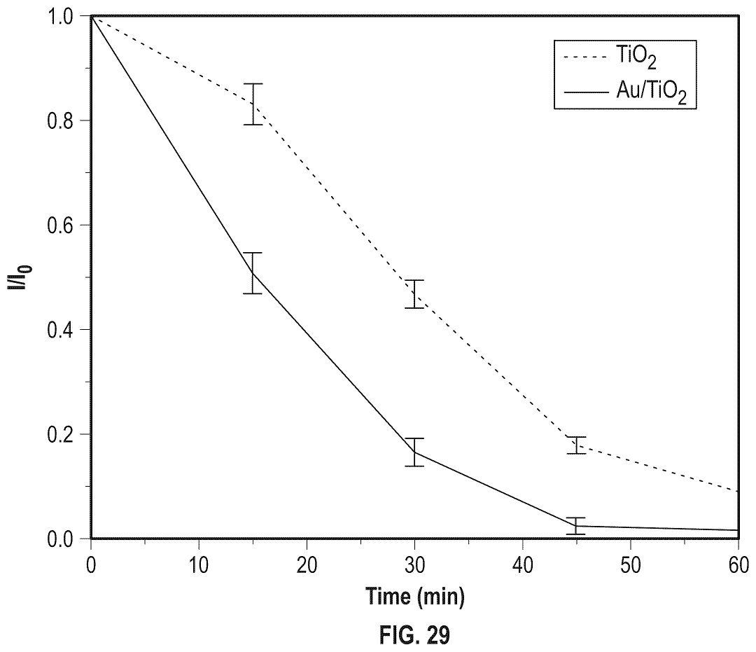

FIG. 29 shows the decrease of UV absorbance at 554 nm of Rhodamine B over time using TiO₂ and Au/TiO₂ as catalysts exposed to UV light, where error bars represent the standard deviation of five repeated experiments.

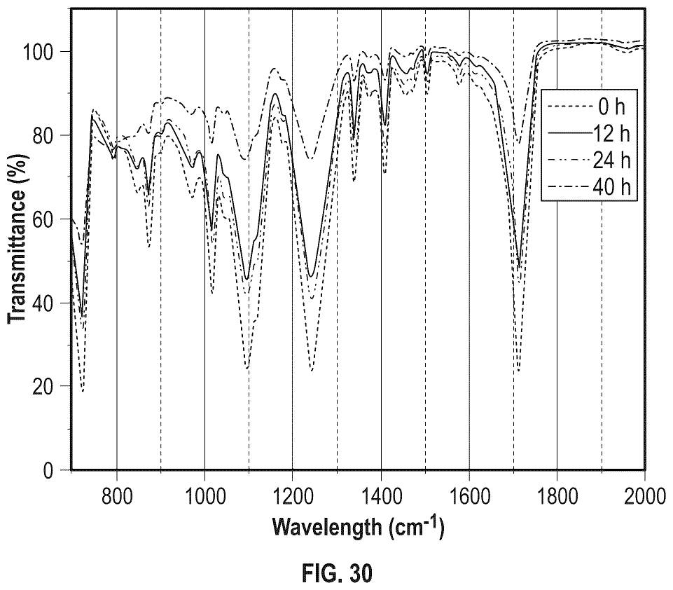

FIG. 30 shows the comparison of FTIR spectra of PET during the photo-reforming process over time. Transmittance % increases over time.

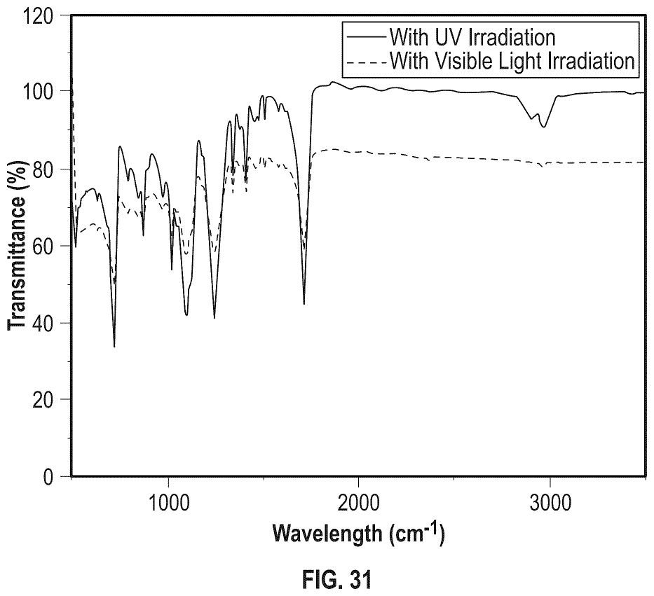

FIG. 31 shows the comparison of PET degradation with Au/TiO₂ under UV and visible light irradiation. Visible light irradiation generally has a lower transmittance % than UV irradiation.

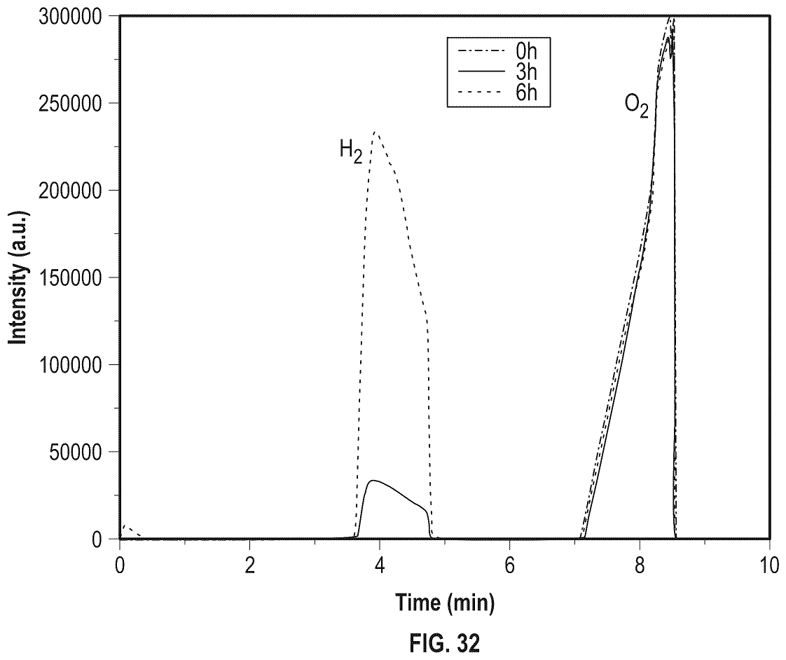

FIG. 32 shows the headspace gas composition analysis with different time steps, using Au/TiO₂ as the photocatalyst. The amount of H₂ is the greatest at the 6-hour time point.

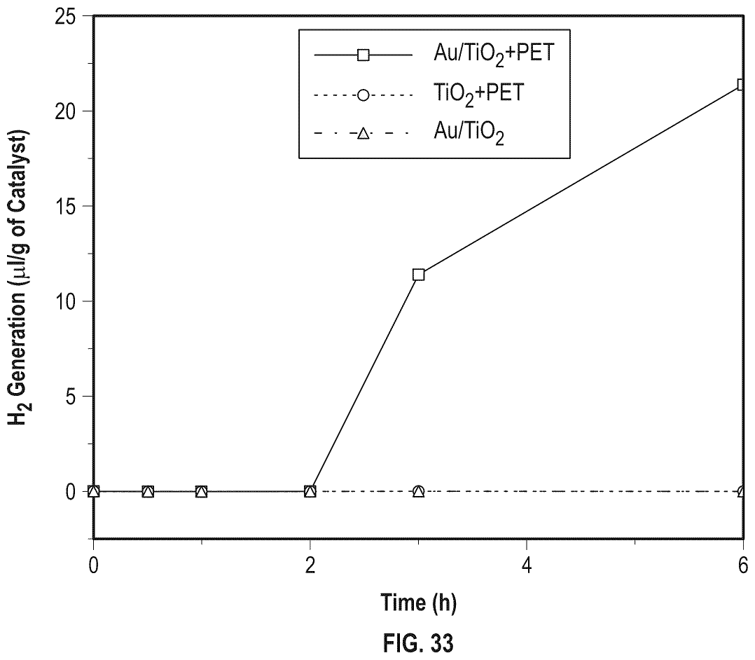

FIG. 33 shows the quantitative analysis of H₂ production over time using Au/TiO₂ and TiO₂ as the photocatalyst. Au/TiO₂ in the presence of PET generates H₂ at the 2-hour timepoint.

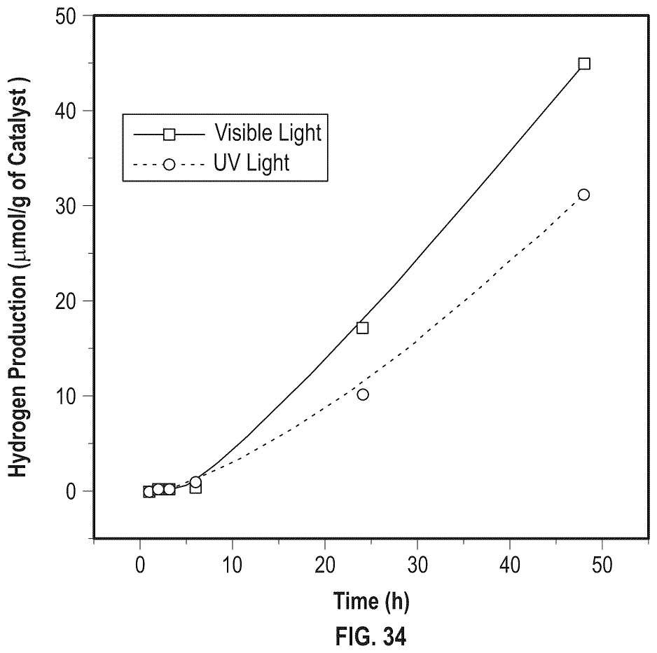

FIG. 34 shows the quantitative analysis of H₂ production using Au/TiO₂ as catalysts for degradation of PET in substrate using a high pressure UV mercury vapor lamp (60 mW/cm²) as a UV light source and a visible light source using a metal halide lamp (45 mW/m²).

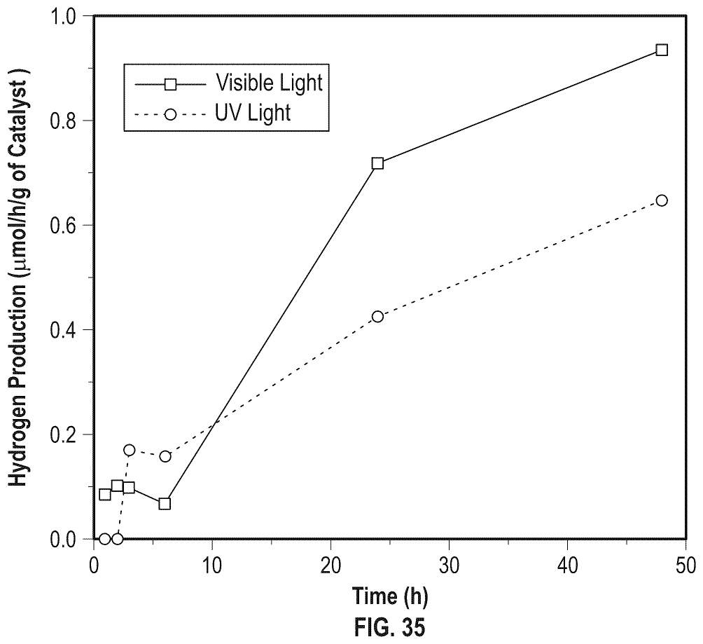

FIG. 35 shows the quantitative analysis of H₂ production rate using Au/TiO₂ as catalysts for degradation of PET in substrate using a high pressure UV mercury vapor lamp (60 mW/cm²) with the visible light source provided by a metal halide lamp (45 mW/m²).

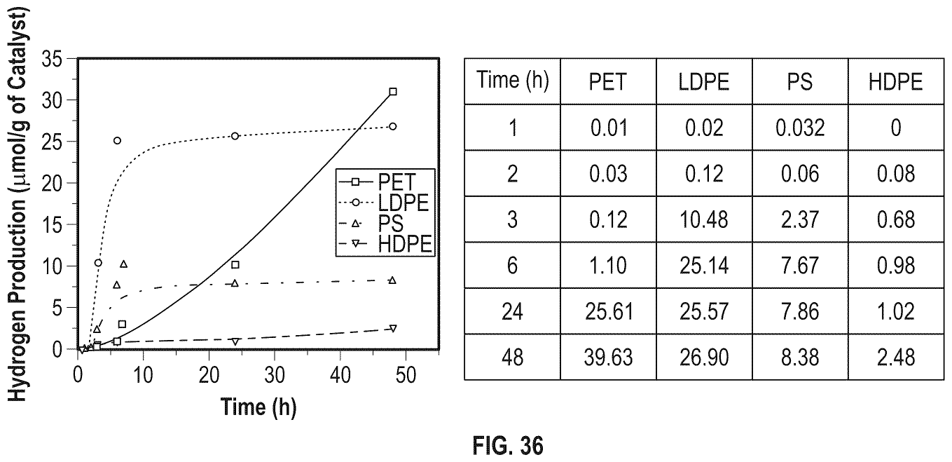

FIG. 36 shows the UV high pressure lamp (60 mW/cm²)-hydrogen yield (μmol/g) for various plastics over time. Hydrogen yields are greatest with PET and LDPE.

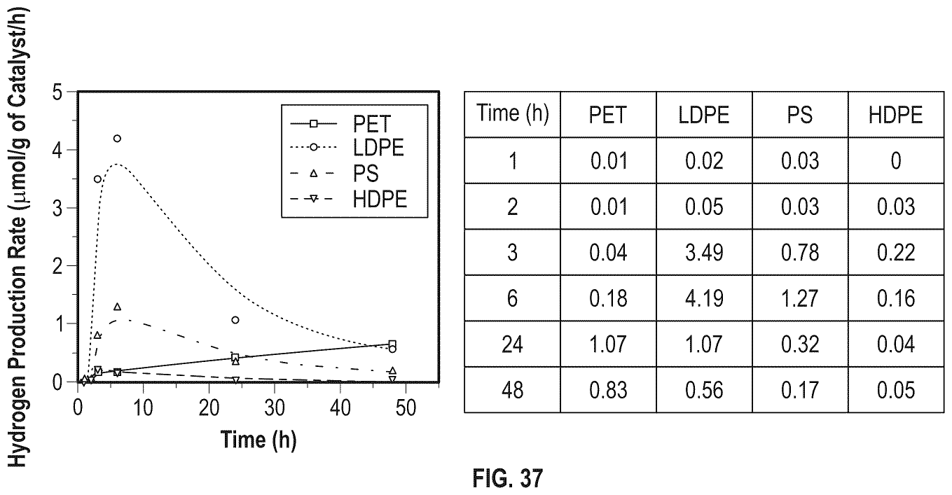

FIG. 37 shows the UV high pressure lamp (60 mW/cm²)-hydrogen production rate (μmol/g/h) over time. Hydrogen production rates are greatest with PET and LDPE.

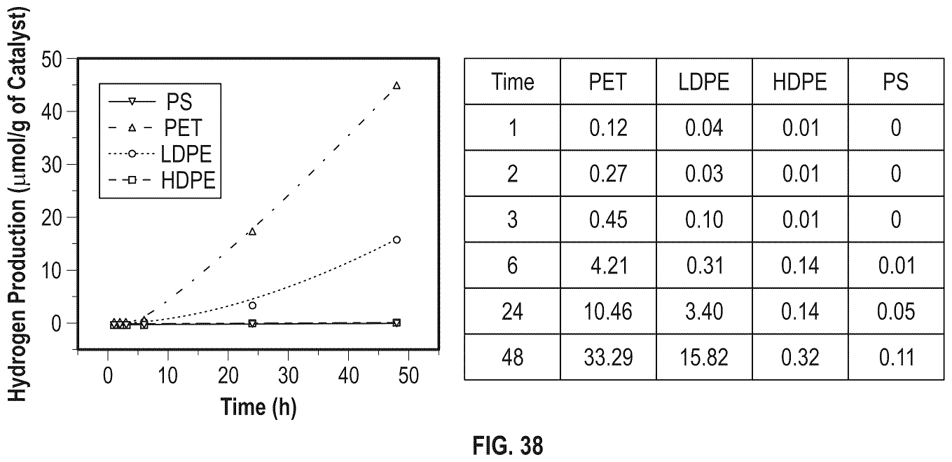

FIG. 38 shows visible light (45 mW/cm²)-hydrogen yield (μmol/g) for various plastics over time. Hydrogen production was greatest with LDPE and PET.

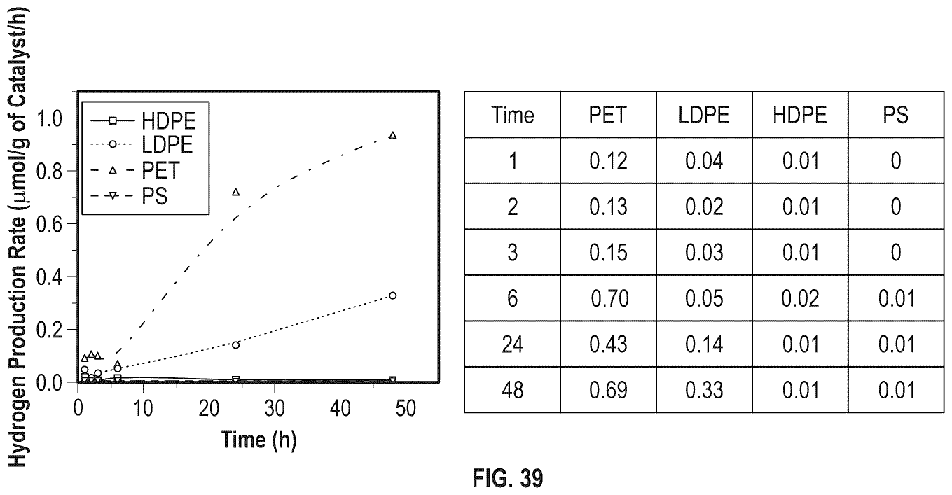

FIG. 39 shows visible light (45 mW/cm²)-hydrogen production rate (μmol/g/h) for various plastics over time. The hydrogen production rate was greatest with PET and LDPE.

The nanocomposite may comprise a metal and semiconductor. The semiconductor may comprise a metal oxide, a metal sulfide, a graphene oxide, a silicon oxide, silicon (“Si”), germanium (“Ge), or a combination thereof. The metal may comprise a precious metal, a noble metal, a base metal, a platinum group metal, or a combination thereof. The metal may include, but is not limited to, gold (“Au”), platinum (“Pt”), copper (“Cu”), iron (“Fe”), or a combination thereof. The metal may be attached to the surface of the semiconductor including but not limited to, the metal oxide, a metal sulfide, a graphene oxide, a silicon oxide, silicon (“Si”), germanium (“Ge”), or combination thereof. The metal may also be disposed into a lattice of the metal oxide, a metal sulfide, a graphene oxide, a silicon oxide, Si, Ge, or combination thereof. The metal oxide may include, but is not limited to, TiO₂, ZnO₂, or a combination thereof. The nanocomposite may include, but is not limited to, Au/TiO₂, Pt/TiO₂, Pt/ZnO, or a combination thereof. The nanocomposite may act as a photocatalyst. The polymer may comprise carbon-hydrogen bonds. The polymer may also comprise polymer chains. The polymer may include, but is not limited to, low-density polyethylene (“LDPE”), high-density polyethylene (“HDPE”), PET, PS, or a combination thereof. The polymer may be derived from plastic waste, including, but not limited to, beverage bottles, low-density polyethylene bags, polystyrene plates, or a combination thereof. The average molecular weight for the polymer may be at least about 20,000 g/mol, about 20,000 g/mol to about 30,000 g/mol, about 21,000 g/mol to about 29,000 g/mol, about 22,000 g/mol to about 28,000 g/mol, about 23,000 g/mol to about 27,000 g/mol, about 24,000 g/mol to about 26,000 g/mol, or about 30,000 g/mol. The polymer may be washed with deionized (“DI”) water and/or ethanol, and/or air-dried before pretreatment.

The radiation may comprise solar radiation, UV radiation, concentrated solar radiation (e.g., solar radiation from a solar concentrator), visible light radiation, or a combination thereof.

The method of producing a nanocomposite catalyst may comprise contacting a precursor with a semiconductor. The precursor may comprise a metal including, but not limited to, H₂PtCl₆·6H₂O. The precursor may be directly deposited onto the semiconductor using a precipitation method and/or dark (e.g., in the absence of light) deposition method. The metal from the precursor may be deposited onto the semiconductor as an individual metal atom or plurality of metal atoms. The precursor may be at a concentration of at least about 0.001 mM, about 0.001 mM to about 0.1 M, about 0.01 mM to about 0.05 mM, or about 0.1 mM. The loading percentages of the metal may be at least about 0.1% (w/w), about 0.1% (w/w) to about 8% (w/w), about 0.3% (w/w) to about 6% (w/w), about 0.7% (w/w) to about 1.5% (w/w), or about 8% (w/w).

The precursor dosage may be controlled to deposit the single metal atoms on the semiconductor surface. High precision and quality in deposition, minimal noble metal loading, and maximal surface-to-volume ratio may be advantages of the dark deposition method. Synthesis under direct deposition may avoid solvent contamination and may produce uniform-quality, pure nanoparticles with high crystallinity.

Dark direct deposition of single atom deposition may comprise heating metal oxide nano powder. The nano powder may be heated to at least about 400° C., about 400° C. to about 600° C., about 450° C. to about 550° C., or about 600° C. The nano powder may be contacted with a MeOH solution comprising a precursor. The nano powder and precursor may be mixed in the dark and may be mixed for at least about 12 hours, about 12 hours to about 36 hours, about 18 hours to about 32 hours, about 22 hours to about 28 hours, or about 36 hours to form a nanocomposite. The nanocomposite may be washed and/or dried. The nanocomposite may be washed with ethanol.

Dark direct deposition may improve the electron storage capacity of the catalyst by trapping electrons. Trapped electrons may reduce the cocatalyst ions into their metallic form and may allow for the growth and aggregation of the deposited single atoms to be controlled. The deposited single atoms may be uniformly distributed onto the semiconductor surface.

The photocatalytic oxidation ability may be improved by adjusting the pH of a solution comprising the polymer and the nanocomposite. The pH may be adjusted to from basic to neutral, acidic to neutral, neutral to basic, neutral to acidic, or a combination thereof when the nanocomposite is in contact with the polymer.

The nanocomposite may produce H₂ at a constant rate for at least about 5 days, about 5 days to about 50 days, about 10 days to about 45 days, about 15 days to about 40 days, about 20 days to about 35 days, about 25 days to about 30 days, or about 50 days. The concentration of hydrogen produced may be at least about 4000 μmol, about 4000 μmol to about 10000 μmol, about 5000 μmol to about 9000 μmol, about 6000 μmol to about 8000 μmol, or about 10000 μmol after five days. The nanocomposite may also produce oxygen. The ratio of hydrogen to oxygen produced may production be at least about 40:1, about 40:1 to about 10:1; about 30:1 to about 20:1, or about 10:1.

Pt⁴⁺ may be reduced to Pt^{δ+} (surface trapped) and Ti³⁺ may be oxidized to Ti⁴⁺. Attached Pt^{δ+} may act as an electron relay, aiding electron trapping and promoting H₂ formation. The semiconductor with attached Pt^{δ+} may act as a catalyst without catalytic loss over time. Pt^{δ+} (approximately Pt²⁺) may act as an electron sink (intermediate Pt red-ox states can act as an electron sink in relation to the TiO₂ conduction band).

Pt⁴⁺+2e⁻→Pt²⁺ (1),

Ti³⁺→Ti⁴⁺+e⁻ (2)

Pt^{δ+}+e⁻→Pt^{δ-1} (3),

Pt^{δ-1}+H⁺→1/2 H₂+Pt^{δ+} (4).

The polymer to nanocomposite ratio may be at least about 0.5:2:5, about 1:2, about 1:1, about 2:1, or about 2.5:0.5. The polymer to nanocomposite ratio may be maintained regardless of polymer type and/or PH level.

The polymer may be pretreated before being contacted with the nanocomposite catalyst. The polymer may be of any size including, but not limited to, at least about 1 μm×1 μm to about 1 m×to 1 m, about 1 mm×1 mm to about 1 dm×to 1 dm, or about 1 μm×1 μm to about 1 m×to 1 m in width and length. The polymer may be pretreated by exposing the polymer to basic conditions. Pretreating the polymer may comprise contacting the polymer with a caustic including, but not limited to, NaOH, KOH, or a combination thereof. The molarity of the caustic may be at least 1 M, about 1 M to about 10 M, about 2 M to about 9 M, about 3 M to about 8 M, about 4 M to about 7 M, about 5 M to about 6 M, or about 10 M. Optionally, the polymer may be pretreated under neutral conditions and/or without contacting the polymer with a caustic. The polymer may be pretreated for at least about 1 hour, about 1 hour to about 48 hours, about 4 hours to about 44 hours, about 8 hours to about 40 hours, about 12 hours to about 36 hours, about 16 hours to about 32 hours, about 20 hours to about 28 hours, or about 48 hours. The polymer may be pretreated in a closed system. The polymer may be pretreated at a temperature of at least about 25° C., about 25° C. to about 700° C., about 50° C. to about 600° C., about 100° C. to about 500° C., 200° C. to about 500° C., 300° C. to about 400° C., or about 700° C. with or without mixing. The pretreated polymer may be kept in the dark until used for photo-reforming by contact with the nanocomposite catalyst and UV and/or visible radiation. Pretreatment of the polymer may break down long-chain polymers.

The polymer may be hydrolyzed by pretreatment. Hydrolysis may occur under alkaline or acidic pretreatment. Hydrolysis of the polymer degrades to polymer to increase hydrogen production by the nanocomposite, which further degrades to the polymer to produce hydrogen.

The polymer may be contacted with a base to be pretreated. The base may be at a concentration of at least about 2 M, about 2 M to about 12 M, about 4 M to about 10 M, about 6 M to about 8 M, or about 12 M. The polymer and/or base may be at a temperature of at least about 20° C., about 20° C. to about 50° C., about 25° C. to about 45° C., about 30° C. to about 40° C., or about 50° C. The polymer may be hydrolyzed by contact with a base.

The polymer may be contacted with ethanol to be pretreated. The polymer may be contacted with ethanol and/or a base including, but not limited to, sodium hydroxide (“NaOH”). The polymer, base, and/or ethanol may be at a temperature of at least about 20° C., about 20° C. to about 30° C., about 21° C. to about 29° C., about 22° C. to about 28° C., about 23° C. to about 27° C., about 24° C. to about 26° C., or about 30° C. The base may be at a concentration of at least about 1% (w/w), about 1% (w/w) to about 10% (w/w), about 2% (w/w) to about 9% (w/w), about 3% (w/w) to about 8% (w/w), about 4% (w/w) to about 7% (w/w), about 5% (w/w) to about 6% (w/w), or about 10% (w/w). The polymer may be hydrolyzed by contact with ethanol.

The polymer may be contacted with an acid to be pretreated. The acid may be at a concentration of at least 0.5 M, about 0.5 M to about 2 M, about 1 M to about 1.5 M, or about 2 M and form a polymer and acid mixture. The pH of the mixture may be adjusted to pH 7 and may be adjusted by adding caustic. The polymer and acid mixture may be contacted with Na₂TP. The Na₂TP may catalyze acidic hydrolysis. The polymer and/or acid may be at a temperature of at least about 20° C., about 20° C. to about 30° C., about 21° C. to about 29° C., about 22° C. to about 28° C., about 23° C. to about 27° C., about 24° C. to about 26° C., or about 30° C. The polymer may be hydrolyzed by contact with an acid.

Pretreated polymers may comprise unmodified and/or more stable functional groups. Bands corresponding to the more stable functional groups, such as the terephthalic group and stretching of the C═O group, may remain the same despite their absorbance changes. The stability of the C═O group may be analyzed by calculating the carbonyl index (CO index). Exemplary analysis is presented in Table 5. The CO index depends on the absorbance of the carbonyl group band and the methylene scission band. The CO index is predicted to increase if degradation occurs during the pretreatment since the formation of new acid end groups and scissoring of CH₂ bonds are expected. Table 5 shows no considerable difference in the carbonyl indexes after pretreatment of the PET compared to virgin PET particles. Therefore, significant degradation is not observed after pretreatment of the PET.

TABLE 5
Calculated carbonyl index for PET

| | Carbonyl | Carbonyl index after |
|---|---|---|
| | index after | photo-reforming for |
| Pretreatment method | pretreatment | 48 hours with Pt/TiO₂ |
| Virgin PET | 2.72 | 5.22 |
| 3M KOH 25° C. | 2.69 | 8.81 |
| 10M KOH 25° C. | 2.62 | 15.89 |
| EtOH-induced 25° C. | 2.65 | — |
| H₂SO₄ 25° C. | 2.72 | 8.21 |
| H₂SO₄ Na₂TP 25° C. | 2.66 | 7.93 |

An Au/TiO₂ nanocomposite may be synthesized by doping a TiO₂ nanoparticle with a gold nanoparticle. The gold nanoparticle may be at least partially disposed on the surface of the TiO₂ nanoparticle using a chemical deposition method. An Au solution may be prepared using chloroauric acid (“HAuCl₄”). The molarity of the Au solution may be at least about 1×10⁻³ M to about 1×10⁻⁷, about 1×10⁻⁴ M to about 1×10⁻⁶, or about 1×10⁻⁴ M. The pH of the Au solution may be at least about 4, about 4 to about 13, about 5 to about 12, about 6 to about 11, about 7 to about 10, about 8 to about 9, or about 13. The pH may be adjusted using caustic including, but not limited to, NaOH. The Au loading and/or doping may be at least about 0.1%, about 0.1% to about 10%, about 0.5% to about 9%, about 1% to about 8%, about 2% to about 7%, about 3% to about 6%, about 4% to about 5%, or about 10% (w/w). TiO₂ nanoparticles may be contacted with the prepared Au solution to form an Au and TiO₂ solution. The Au and TiO₂ solution may be at a concentration of at least about 0.1 g/50 ml, about 0.1 g/50 ml to about 10 g/50 ml, about 0.5 g/50 ml to about 9 g/50 ml, about 1 g/50 ml to about 8 g/50 ml, about 2 g/50 ml to about 7 g/50 ml, about 3 g to about 6 g/50 ml, about 4 g/50 ml to about 5 g/50 ml, or about 10 g/50 ml. The Au and TiO₂ solution may be stirred and/or maintained at a temperature of about 293 K. The pH of Au and TiO₂ solution may be about 8 to about 10, or about 9. The Au and TiO₂ solution may be formed into a suspension and be left at room temperature and optionally stirred for up to 24 hours. The Au and TiO₂ suspension may also be heated to at least about 300 K, about 300 K to about 575 K, about 325 K to about 550 K, about 350 K to about 525 K, about 375 K to about 500 K, about 400 K to about 475 K, or about 575 K, and optionally stirred for up to 24 hours to form the Au/TiO₂ nanocomposite. The Au and TiO₂ suspension may be cooled to the room temperature, filtered, and solids may be removed with a solvent including, but not limited to, deionized water. The filtered and washed Au and TiO₂ suspension may be dried, e.g., by vacuum-drying for up to 48 hours or until dry. The drying may occur at room temperature.

A Pt/TiO₂ nanocomposite may be synthesized by doping a TiO₂ nanoparticle with a platinum nanoparticle. The platinum nanoparticle may be at least partially disposed on the surface of the TiO₂ nanoparticle using a chemical deposition method. A Pt solution may be prepared using H₂PtCl₆ solution. The molarity of the Pt solution may be at least about 1×10⁻³ M to about 1×10⁻⁷, about 1×10⁻⁴ M to about 1×10⁻⁶, or about 1×10⁻⁴ M. The pH of the Pt solution may be at least about 7, about 7 to about 12, about 6 to about 11, about 7 to about 10, about 8 to about 9, or about 12. The pH may be adjusted using caustic including, but not limited to NaOH. The Pt loading and/or doping may be at least about 0.1%, about 0.1% to about 10%, about 0.5% to about 9%, about 1% to about 8%, about 2% to about 7%, about 3% to about 6%, about 4% to about 5%, or about 10% (w/w). The Pt solution may be contacted with dry TiO₂ to wet the TiO₂ to form a Pt and TiO₂ slurry. The slurry may be left at room temperature and optionally stirred for up to 24 hours. The Pt and TiO₂ slurry may also be heated to at least about 300 K, about 300 K to about 575 K, about 325 K to about 550 K, about 350 K to about 525 K, about 375 K to about 500 K, about 400 K to about 475 K, or about 575 K, and optionally stirred for up to 24 hours to form the Pt/TiO₂ nanocomposite. The slurry may be dried. Drying may occur at a temperature of at least about 30° C., about 30° C. to about 75° C., about 35° C. to about 70° C. about 30° C. to about 50° C. about 30° C. to about 50° C. for up to 24 hours or until dry. Drying may be performed by an air oven, vacuum desiccator, or a combination thereof.

A Pt/ZnO nanocomposite may be synthesized by doping a ZnO nanoparticle with a platinum nanoparticle. The platinum nanoparticle may be at least partially disposed on ZnO nanoparticle surface using a chemical deposition method or within a lattice of ZnO nanoparticles. A reaction solution may be prepared by contacting zinc acetate (“Zn(CH₃COO)₂”)·2H₂O with methanol at a temperature of up to 60° C. A methanolic solution of KOH may be added to the reaction solution to form ZnO nanoparticles. A Pt solution may be prepared using H₂PtCl₆ solution. The Pt solution may be added to the ZnO dispersed nanoparticles. The Pt and ZnO solution may be contacted with a sodium borohydride (“NaBH₄”) aqueous solution to form the Pt/ZnO nanocomposite.

Zn-doped TiO₂ nanoparticles, e.g., the nanocomposite, may also be synthesized by sol-gel method. The sol-gel method may comprise forming a solution of ethanol (“EtOH”) and isopropanol and contacting the solution with titanium (IV) butoxide. The solution may then be contacted with an aqueous solution of zinc nitrate hexahydratecetyltrimethylammonium bromide (“CTAB”) to form a nanocomposite. Heat may be applied to the solution and excess solution may be evaporated from a water bath with continuous stirring. The nanocomposite may then be dried.

The method may produce a byproduct. The byproduct may comprise disodium terephthalate (“Na₂TP”) and terephthalic acid (“TPA”) after the pretreatment of a polymer. The byproducts may be extracted and re-applied in the photo-reforming (hydrogen production) method. Hydrogen may be produced from byproducts acting as polymers according to the methods described herein. The byproduct may be at a concentration of at least about 35 mg/g, about 35 mg/g to about 75 mg/g, about 40 mg/mg to about 70 mg/g, about 45 mg/g to about 65 mg/g, about 50 mg/g to about 60 mg/g, or about 75 mg/g of polymer.

The semiconductor photocatalysts are capable of absorbing visible light and producing H₂ from water. Titanium oxide (TiO₂) is a promising catalyst with broad applications, but it requires UV light to activate the redox reaction process. Au and Pt are considered as promising doping materials for TiO₂ to produce H₂ from water in the photo-reforming process. Light flux may be measured using a solar power meter.

The nanocomposite may degrade a polymer in the presence of radiation. The surface morphology of the polymer may be changed to display cracks that may increase with increased irradiation and/or time. The polymer edges may be eroded due to catalytic activity. A change of color of the polymer may occur and/or the transparency of the polymer may be reduced.

The size of the semiconductor including, but not limited to, the metal oxide, a metal sulfide, a graphene oxide, a silicon oxide, or combination thereof, may be at least about 8 nm, about 8 nm to about 50 nm, about 10 nm to about 45 nm, about 15 nm to about 40 nm, about 20 nm to about 35 nm, about 25 nm to about 30 nm, or about 50 nm. The size of the metal may be at least 1 atom, about 1 atom to about 10 nm, about 1 nm to about 9 nm, about 2 nm to about 8 nm, about 3 nm to about 7 nm, about 4 nm to about 6 nm, or about 10 nm.

A polymer in the presence of the nanocomposite catalyst may be contacted with visible and/or UV radiation. Polymer contacted in the presence of the nanocomposite catalyst and contacted with visible and/or UV radiation may generate hydrogen (“H₂”), oxygen (“O₂”), nitrogen (“N₂”), or a combination thereof. H₂ may be generated after visible and/or UV radiation.

Embodiments of the present invention provide a technology-based solution that overcomes existing problems with the current state of the art in a technical way to satisfy an existing problem for plastic waste removal from aqueous solutions and for hydrogen production. Embodiments of the present invention achieve important benefits over the current state of the art, such as plastic waste removal, polymer degradation, and/or hydrogen production at ambient temperature and pressure using solar light. Some of the unconventional steps of embodiments of the present invention include a nanocomposite with individual metal atoms attached to a semiconductor and method using a nanocomposite to degrade polymers while simultaneously producing hydrogen gas with polymer pretreated in dark conditions.

### EXAMPLES

The invention is further illustrated by the following non-limiting examples. The materials and methods used in examples 1 to 21 are summarized in Table 6 and further discussed in the following sections.

TABLE 6
Summary of the methodology.

| Step | Description |
|---|---|
| 1. Synthesis of feasible | Au/TiO₂ |
| catalysts | Pt/TiO₂ |
| | Pt/ZnO |
| 2. Evaluation of | Rhodamine B degradation with |
| photocatalytic activity | time was measured using UV-Vis |
| | spectrophotometer |
| 3. Characterization of | TEM |
| Au/TiO₂ and pure TiO₂ | X-ray diffraction (“XRD”) |
| | UV-Vispectrophotometer |
| | ATR-FTIR |
| | XPS |
| 4. Analysis of plastic | Optical microscopy |
| degradation | ATR-FTIR |
| 5. Analysis of headspace | Gas chromatography-thermal |
| gas | conductivity detector (“GC-TCD”) |

Table 6 shows the methods used including, but not limited to, PET, transmission electron microscopy (“TEM”), X-ray diffraction spectroscopy (“XRD”), attenuated total reflectance furrier transformation spectroscopy (“ATR-FTIR”), X-ray photoelectron spectroscopy (“XPS”), and gas chromatography with thermal conductivity detector (“GC-TCD”).

All the materials used in examples 1 to 21 are analytical-grade chemicals. Details of the chemicals are listed in Table 7.

TABLE 7
Summary of materials used.

| Chemical | Description |
|---|---|
| 1. Hexachloroplatinic acid | 8 wt. % in H₂O |
| solution (“H₂PtCl₆”) | |
| 2. Sodium Borohydride | 12% aq. solution |
| (“NaBH₄”) | |
| 3. Chloroauric acid | 99.99% (metals basis), Au 49.5% min |
| (“HAuCl₄”) | |
| 4. Zinc Acetate Dihydrate | 219.51 g/mol |
| (Zn(CH₃COO)₂ · 2H₂O) | 1.74 g/cm³ at 20° C. (68° F.) |
| | (anhydrous substance) |
| 5. Methanol | 0.791 g/mL at 25° C. (77° F.) |
| 6. Potassium hydroxide | 56.11 g/mol |
| (KOH) | 2.04 g/cm³ at 20° C. (68° F.) |
| 7. Titanium (IV) oxide | nanopowder, 21 nm primary particle |
| (TiO₂) | size (TEM), ≥99.5% trace metals basis |
| 8. NaOH | 2.13 g/cm³ at 20° C. (68° F.) |
| 9. Rhodamine B | 9-(2-Carboxyphenyl)-3,6 |
| | bis(diethylamino)xanthylium chloride |
| | <=100 % |

### Example 1

An Au/TiO₂ catalyst was synthesized. Gold nanoparticles were doped on a TiO₂ nanoparticle surface using the chemical deposition method. The 1×10⁻⁴ M Au solution was prepared using HAuCl₄ (99.99% assay), and its pH was adjusted to 13 (from 4) by using a 0.5 M NaOH solution. TiO₂ nanoparticles were added to the prepared Au solution until the Au loading was 5% (w/w). The Au and TiO₂ solution (1 g/50 ml solution) was stirred at 300 RPM using a magnetic stirrer and the temperature was maintained at 293 K. The pH was monitored before and after adding the TiO₂ for each sample preparation. The final pH was maintained at 9 by adjusting the use of the 0.5 M NaOH solution. The resulting suspension was heated to 343 K and vigorously stirred at 300 RPM for 1 hour. After cooling the solution to the room temperature the solution was filtered and the solids were washed thoroughly with 2 L of deionized water (DI) and then vacuum-dried at room temperature for 24 hours.

### Example 2

A Pt/TiO₂ catalyst was synthesized. Hexachloroplatinic acid solution (H₂PtCl₆) with a molarity of 1×10⁻⁴ M was prepared and solution PH was raised to 9 by adding 3M NaOH. The 50 ml of the salt solution of the metal for doping was added to the 1 g of the catalyst (P-25 TiO₂) in such a way that they wet the catalyst completely. The slurry was stirred for 24 hours at ambient temperature and dried in an air oven at 50° C. for 12 hours. The dried powder was then cooled to room temperature and the prepared catalysts were stored in a vacuum desiccator.

### Example 3

A Pt/ZnO catalyst was synthesized. In a typical synthesis procedure, Zn(CH₃COO)₂·2H₂O (4 mmol) was added to methanol (70 mL) in a 100 mL round bottom flask, and the temperature was raised to 60° C. After a few minutes, methanolic solution of KOH (500 mg in 10 mL) was added dropwise to the reaction solution and the stirring (300 rpm) was continued for 2 hours at 60° C. The color of the solution became turbid at the initial stages and became colorless in 30 minutes. In 2 hours, the solution slowly turned to a white color (the particle size also depends on the size of the magnetic bead and rpm of stirring). Formed ZnO nanoparticles were precipitated out by the addition of water and excess zinc ions were removed by centrifugation. For loading of platinum nanocrystals, 2.3 ml of (8.66 mM) H₂PtCl₆ aqueous solution was added dropwise to the preformed ZnO nanocrystals dispersed in a fresh methanolic solution, followed by 0.5 mL of NaBH₄ (40 mM) aqueous solution. The solution was stirred for 10 minutes and the solution color became black due to the formation of platinum on ZnO nanoparticles. Excess zinc ions were removed by centrifugation and the solid product was separated and used in the photodegradation experiment after drying them at 50° C. for 24 hours.

### Example 4

The photocatalytic activity of catalysts was evaluated. Photocatalytic activity of the prepared catalysts was measured using 30 mg/L Rhodamine B solution. Rhodamine B was used as an indicator organic compound which originally gives a pink color and is colorless after degradation. About 0.05 g of catalysts were suspended in 50 ml of DI water and ultrasonicated for 10 minutes. 10 ml of Rhodamine B solution (30 mg/L) was then added into the suspensions and irradiated by UV light for 1 hour. 10 ml samples were collected in 15-minute intervals. Change of the solution color as an indicator of photocatalytic degradation of the dye solution was quantified using an ultraviolet-visible spectrophotometer (UV-vis). The absorbance at 554 nm was measured to quantify the Rhodamine B degradation using a single wavelength scan of the UV-vis spectroscopy.

The structure and morphology of the TiO₂ and TiO₂ doped with Au were characterized by a TEM. The samples for examination by TEM were prepared by placing the dry catalyst powder on a holey carbon film supported by a 300-mesh copper TEM grid. TEM imaging showed the shape, size, lattice fringes and calculation of particle size histograms of Au/TiO₂. TEM analysis also allowed for the measurement of the nano-particle size. This can be very useful for the characterization of a catalyst where particle size is a key factor. Once the nanoparticle's diameter is determined, a particle size histogram can be created which displays the number and size of particles of each size, as well as allow for the generation of average particle size and standard deviation.

### Example 5

XRD technique is based on the Bragg's law:

$$n λ = 2 d sin θ , \qquad ( 5 )$$

where λ corresponds to the wavelength of the x-ray radiation, d is the interplanar distance, and θ is the angle of diffraction. XRD technique was used to determine the composition of the TiO₂ sample using the characteristic peaks given for different phases of TiO₂ and to clarify any other components present using their respective peaks. Change in the peak intensities, peak areas, as well as the absence of certain peaks, indicated the crystal structure of TiO₂ and its surface modification with Au. Intercalated structures resulted in increased d spacing. This caused the angle θ to shift to lower values due to the inverse relationship with d. In contrast, the Au doping on TiO₂ structures was characterized by the presence of new peaks.

Characterization was performed using a CuKα radiation (λ=1.54 Å) XRD diffractometer. One gram of TiO₂ and Au/TiO₂ samples were placed on the cradle using a glass slide with double sticky tape and scanned at 0.039 deg/s rate in reflection over the range of 2θ=5°-80°. The intensity/CPS was plotted against the diffraction angle (20) in degrees. The d values for peaks were calculated using the VESTA software. The resulted XRD spectra were compared with the data available in the literature.

### Example 6

A wavelength scan of the catalysts (Au/TiO₂ and pure TiO₂) within the range of 190 nm to 800 nm was done. 0.05 g of catalyst was dispersed in 50 ml of DI water and a full wavelength scan was done using UV spectrophotometer. Depending on the wavelength scan results, bandgap analysis was done, thus the feasibility of the catalytic activity was evaluated. Tauc plot method was used in the calculations and analysis.

### Example 7

XPS analysis of the P25 TiO₂ and Au/TiO₂ nanoparticles was conducted. All survey spectra were collected in constant analyzer energy mode with an analyzer pass energy of 160 eV. Individual regions were collected at an analyzer pass energy of 20 eV using the hybrid lens mode which features a combination of electrostatic and magnetic immersion electron collection optics. The maximum analysis area is 700 μm×300 μm using the slot aperture. Photoelectrons are amplified and detected using a microchannel plate stack in a z-configuration with a position-sensitive delay-line detector divided into 127 channels. Typical base pressure during analysis is <5×10⁻⁹ torr. Charge compensation/neutralization of surface charging was done using a low kinetic energy electron flux to the surface of the sample.

All fittings were done using processing software. Background subtraction was done using linear or integral backgrounds as well as peak fitting typically using a combination of Gaussian and Lorentzian hybrid models. Samples were embedded in carbon tape in a thin layer to maximize contact with the carbon. Some charge shift is in the raw data but the C 1s was used to correct the energy scale to C 1s at 284.6 eV binding energy (adventitious carbon).

Characterization of plastic degradation using ATR-FTIR was performed. In FTIR transmittance bands were observed due to molecular vibrations. These vibrations are of two types, stretching and bending. FTIR spectra for plastics vary from before and after the reaction. Therefore, it was used as a fingerprint to identify the type of reaction that plastic undergoes. Transmittance bands were observed for various vibrations that are characteristic of the plastics and the new band that occurs due to the photo-reformation. This reflected through various bands characteristic to the functional groups in the material.

### Example 8

The IR spectra of TiO₂ and Au/TiO₂ were obtained. All samples were well powdered and placed on the crystal and the compression clamp was used to obtain good contact between the sample and the crystal to obtain the spectra.

### Example 9

Plastic polymers were pre-treated. The PET particles of size 2 mm×2 mm were used as the polymer in the experiments. The 50 mg of PET was soaked in 1.0 ml of 2M KOH solution in a sealed vial for 24 h at 40° C. with stirring at 300 RPM. The solution was kept in the dark until used for photo-reforming as below.

### Example 10

A photo-reforming process was performed. Five hundred mg of catalyst was dispersed in 50 ml of DI water and ultrasonicated (pulses of 30 s at 100% amplitude followed by 5 s paused) for 10 minutes. Then 500 mg of pre-treated polymer in 3 M aqueous semiconductor grade KOH was added to the sample. The temperature was maintained at 273 K and pH was maintained at 9. Equations 7 and 8 describe the reactions of the PET hydrolysis and photo-reforming of EG.

PET hydrolysis: C₁₀H₆O₄+2 H₂O→C₂H₆O₂+C₈H₆O₄,ΔG°=66 KJ mol⁻¹ (6),

Photo-reforming of EG: C₂H₆O₂+2 H₂O→5H₂+2 CO₂,ΔG°=9.2 KJ mol⁻¹ (7).

A 50 ml quartz round bottom flask was used in UV irradiation and the used light sources include a high-pressure UV mercury vapor lamp (160 W PUV-10), a low-pressure UV lamp (39 W T5), and a visible light source provided by a fluorescent lamp (40 W F40T12/DX). The irradiance of the high-pressure UV lamp concentrates on both UV (minor peaks at 290 nm, 315 nm, 335 nm, and a dominant peak at 365 nm) and visible light (405 nm, 435 nm, and 545 nm) wavelength ranges. The irradiance of a low-pressure UV lamp is primarily at 253.7 nm. The experiment was carried out for 60 hours.

### Example 11

Plastic degradation was characterized. The plastic degradation and characterization of PET were carried out using an optical microscope and FTIR analysis. Surface morphology of the plastics before and after UV irradiation was observed at 4×, 10×, 20×, and 40× magnification.

### Example 12

The accumulation of H₂ was measured via a gas chromatograph equipped with a thermal conductivity detector and HP-5 molecular sieve column using N₂ as the carrier gas. The instrument was calibrated using the known source of pure H₂. Then 1 mL of headspace gas was directly injected into the inlet of the gas chromatograph. Purge time was selected as 1 minutes and chromatography was collected for 10 mins after injection.

### Example 13

Photo-degradability of different catalysts were evaluated. Photocatalytic activity of synthesized Au/TiO₂, Pt/TiO₂, and Pt/ZnO was tested to evaluate their performance under UV light. Rhodamine B solution was used as an indicator organic compound and absorbance of the samples was measured after UV irradiation. Results were compared with the degradation of Rhodamine B when unmodified TiO₂ was used as the catalyst under similar conditions.

Photocatalytic activity of modified TiO₂ with Au deposition was higher than the other catalysts. Degradation efficiency was the highest in Au/TiO₂, that is 98%, and pure TiO₂ and Pt/TiO₂ had similar degradation efficiency of 91%. Rhodamine B degradation efficiency with Pt/ZnO was 89% which is the lowest compared to other photo activities. With these results, the Au/TiO₂ catalyst was considered for further characterization.

### Example 14

The catalyst Au/TiO₂ was characterized. The TiO₂ before and after modification with Au was observed. At a glance, the TiO₂ was a white-colored powder, and due to the Au doping, the TiO₂ changed the color into pinkish purple.

TEM analysis of the catalyst was performed. The particle size analysis and morphological study of the surface-modified Au/TiO₂ and pure TiO₂ were carried out using TEM with 100 k, 200 k, and 300 k magnification. The presence of the Au particles on the TiO₂ surface confirms the successful surface modification carried out.

The average size of TiO₂ nanoparticles before surface modification with Au was 20.34 nm, and the particle size increased to 20.52 nm after carrying out the surface modification. Hence, the surface modification did not affect the photocatalyst size significantly. The P25-TiO₂ was known to comprise small spherical anatase crystallites and larger angular rutile crystallites. The anatase crystallite particle size varied from 8 nm to 34 nm while the particle size varied from 34 nm to 50 nm for rutile crystallites. Therefore, the TEM results showed that the majority of the P25-TiO₂ sample was anatase but, both crystalline phases of anatase and rutile TiO₂ were present.

The average Au particle size deposited on TiO₂ surface is 2.1 nm and the particle sizes range from 1 nm to 3.5 nm. The Au particles doped vary from 1 nm to 5 nm and exhibit higher photo-, electro-, as well as chemical catalytic activity than larger-sized particles. The Au nanoparticles of the size of 2 nm to 5 nm showed especially high activity for oxidation of CO and propylene. Hence, the doped Au particles were expected to enhance the photocatalytic activity of TiO₂. At pH 9, Au particles are exceedingly small (2 nm) compared to those formed at pH 6 (10 nm). The main reason for the larger size at the lower pH is the retention of chloride ions in the gold complex that exists in solution at this pH and that are attached to the surface in some way or other. Such species are more mobile than those at higher pH that is devoid of chlorine. The Au nanoparticles aggregate into larger clusters during drying and thereby form quite big particles of metallic Au.

### Example 15

XRD analysis of TiO₂ and Au/TiO₂ was performed. The XRD data for TiO₂ and Au/TiO₂ was smoothed using a Savitzky-Golay (“SG”) filter. The SG used a process known as convolution, which fits successive subsets of adjacent data points with a low-degree polynomial by the method of linear least squares.

TABLE 8
Comparison of peak 2θ positions (in °)
of TiO₂ with reported values.

| Observed | Crystalline phases | | Reported |
|---|---|---|---|
| peak | Anatase | Rutile | values (L. Lin |
| positions | (A) | (R) | et al., 2015) |
| 25.10 | 25.3 | | 25 A |
| 27.20 | | 27.4 | 27 R |
| 35.95 | | 36.0 | 36 R |
| 37.69 | 37.7 | | |
| 40.92 | | 41.2 | 41 R |
| 47.83 | 48.0 | | 48 A |
| 53.75 | 53.8 | | 53 R |
| 54.86 | 55.0 | | 55 A |
| 62.57 | 62.6 | | 62 A |
| 68.80 | 68.8 | | |
| 70.00 | 70.2 | | |
| 74.89 | | 74.4 | |

The A and R in the reported value column in Table 8 are the reported anatase crystalline phases and rutile crystalline phases respectively.

The characteristic peaks of anatase are at 25.10°, 37.79°, 48.03°, 53.88°, 55.05°, and 62.68° indexed to (101), (004), (200), (105), (211), and (204) crystal faces, respectively (see, Table 13). The diffraction peak at 20 angles of 27.0° is assigned to the (110) plane of TiO₂ rutile crystalline phases. Hence, the TiO₂ XRD results were consistent with the TEM particle size analysis results such that the TiO₂ contained both anatase and rutile crystalline phases. Furthermore, the XRD results of TiO₂ were in good agreement with the reported values of anatase and rutile crystalline phases results (see, Table 13).

The XRD patterns for the Au/TiO₂ were dominated by the peaks of anatase and rutile crystalline phases of TiO₂ due to the low Au loading (˜ 5%). The TiO₂ weight fraction of each phase in all samples was determined to be 85% (w/w) of anatase and 15% (w/w) of rutile crystalline phases. For the Au/TiO₂ samples, additional broad and weak signals were detected at 38.2, 44.3, 65.6, and 78.9 2θ values that intensified with the nominal Au loading (˜ 5% (w/w)). The position and relative intensity of 38.2°, 43.4°, 65.6°, and 79.8° peaks were consistent with face-centered cubic (“fcc”) Au particles and assigned to Au (111), Au (200), Au (220), and Au (311) reflections, respectively. Hence, the Au/TiO₂ retained an fcc structure since the particle size is close to 2 nm as found in TEM analysis. Furthermore, due to the Au doping, 43.4°, 65.6°, and 79.8° new peaks were observed in Au/TiO₂, confirming the successful doping of Au nanoparticles.

### Example 16

Absorbance spectroscopy analysis of TiO₂ and Au/TiO₂ was conducted. The UV-Vis absorption spectrum of TiO₂ showed the maximum absorption at 345 nm, indicating the TiO₂ nanoparticles had photocatalytic activity in the UV region. However, due to the Au doping, the absorption maxima is shifted to 318 nm and a secondary peak occurs at 586 nm visible region. The absorption maxima shift confirms that Au doping reduced the bandgap of TiO₂ since bandgap is inversely proportional to the wavelength according to equation (9). The absorption wavelength is Amax:

$$Egap = hc λ max , \qquad ( 9 )$$

where Egap is the bandgap between VB and CB, h is Planck's constant, and c is the speed of light. The bandgap analysis was carried out using the Tauc method, which showed that the bandgap energy of pure TiO₂ is around 3.1 eV and due to the Au doping, the bandgap was reduced to 2.6 eV.

The additional absorption band of the surface modified TiO₂ samples (Au/TiO₂) corresponded to the purple color of the photocatalysts. Such a color matrix obtained by Au/TiO₂ was due to the plasmonic absorption band. The surface plasmon absorption band was produced by the movement of the conduction electrons in the particles as a consequence of the incident electric field light, which resulted in a displacement of the negative and positive charges in the metal. The peak maximum corresponding to the plasmon excitation of gold typically occurs around 520 nm in organic solvents. The peak redshift occurred because the gold cluster surface was covered with an oxide layer, which showed that the TiO₂ nanoparticles undergo chemisorption due to the Au doping. Furthermore, the surface plasmon absorption of Au nanoparticles deposited on TiO₂ surface has a redshift compared to that of Au nanoparticles immersed into water. The different dielectric constant of the environment surrounding the Au nanoparticles causes the redshift. Hence, the absorption, typical of well-separated Au nanoparticles in a colloidal suspension, was particularly evident in the absorption spectrum of Au/TiO₂ exhibiting a flatter absorption pattern extending in the visible region.

FTIR analysis of TiO₂ and Au/TiO₂ was performed. The surface modification of TiO₂ with Au resulted in a new band, which occurs in the positions at 3808, 2941, and 1635 cm⁻¹. The band occurring at 3308 cm⁻¹ corresponds to the hydrogen-bonded hydroxyl groups on the surface of the catalyst and is prominently observed in the Au-loaded catalysts. Also, the wavenumbers at 2852 to 2941 cm⁻¹, the bands resulting from the water crystallization, were seen in the Au/TiO₂, while they were not observed in the unmodified TiO₂ catalyst. The surface adsorbed water is responsible for the band observed to 1635 cm⁻¹ in the Au-loaded TiO₂. The observed band in TiO₂ and Au/TiO₂ at 500 cm-1 and 533 cm⁻¹ corresponded to the Ti—O stretching and Ti—O—Ti bridging vibrations respectively. All bands were broadened as a result of surface modification TiO₂. The loading of Au nanoparticles caused surface defects which resulted in broader bands in the vibration and bending modes.

### Example 17

XPS analysis was performed. XPS analysis was used to determine the chemical state of the catalyst before and after gold doping. The C 1s peak at 286 eV was due to adhesive tape used in sample mounting in XPS measurements. The peak at 531.3 eV indicated the O state (“O 1s”) of the crystal and it was assigned to oxygen in Ti—O—Ti. Binding energies of 567 eV, 449, 61, and 39, were assigned to Ti states of 2s, 2p, 3s, and 3p respectively. These peaks were observed in both spectra. Peaks for Au nanoparticles were found at 83.2 and 87.1 eV, corresponding to Au 4f7/2 and Au 4f5/2, indicating a slight shift of the Au 4f peaks toward lower binding energies compared with the peaks located at 84.0 and 87.7 eV, respectively, which is due to the intimate contact between the Au and the TiO₂ substrate which led to a change in the electronic properties. Only the Au 4f peaks and no oxidized Au peaks were seen, which suggested the Au deposited is in a metallic state on the TiO₂. The amount of Au measured by XPS was 1.5% by atomic concentration and 5% by mass loading. XPS spectra results confirmed the Au loading on the TiO₂ lattice and mass percentage was the same as expected in the synthesis process.

### Example 18

The photocatalytic activity was evaluated. To investigate the photocatalytic activity, the Au/TiO₂ contained Rhodamine B (“RB”) solution was exposed to UV radiation and the remaining RB concentration was analyzed by finding the absorption at wavelength 554 nm (Amax for RB). The maximum absorption wavelength for RB was obtained by a full wavelength scan in the range of 450 nm to 650 nm. To confirm the Amax for RB, full wavelength scan was carried out before and after exposure to the UV light. The full wavelength scan of RB with Au/TiO₂ catalyst showed the decrease of absorption with increasing exposure time, which led to an increase in RB degradation, verified by the decreasing concentration. The RB absorption results for Au/TiO₂ catalyst were compared against TiO₂ under similar conditions to evaluate the efficiency and effectiveness of the catalysts. The RB absorption results indicated that photocatalytic activity of modified TiO₂ with Au deposition was higher than the TiO₂ catalysts. A color change was observed, and pink colored solution become colorless with time.

The experiment was repeated 5 times for verification of the results. The standard deviation of testing trials was 0.1157 and 0.0495 for TiO₂ and Au/TiO₂, respectively. The concentration of RB decreased gradually to a value near zero after 1 h proportional to absorbance. The degradation efficiency of Au/TiO₂ was higher at 98% (w/w) compared to TiO₂. The observed degradation rate constant (kobs) was 0.0723 minute⁻¹ for Au/TiO₂ and 0.0369 minute⁻¹ for TiO₂. Hence, the results indicated Au/TiO₂ had higher photocatalytic activity over TiO₂, concluding that the Au doping to TiO₂ surface enhanced the photocatalytic activity of TiO₂.

### Example 19

Microscopic images of PET were taken. The optical microscope was used to analyze the surface changes during the photo-reforming process. The surface morphology of PET was examined at 0 hours, 12 hours, 24 hours, 40 hours, 60 hours of the photo-reforming process, under 4 times, 10 times, 20 times, and 40 times magnifications. The surface morphology of PET plastics changed over time and cracks were observed in PET particles. Crack density and depth of cracks increased with irradiation time. Edges of the plastics were eroded due to catalytic activity. A slight change of color of the plastics was observed and the transparency of the PET was reduced with UV exposure.

### Example 20

FTIR spectrum analysis of PET particles was conducted. High polymer materials such as plastics, rubber, and fibers underwent accelerated oxidative degradation due to the photo-reforming process. This degradation was manifested as an accelerated weakening of the mechanical and/or dielectric strength of the material. Since the oxidized sites had characteristic signatures in the infrared region, FTIR spectroscopy was used as an effective means for investigating the state of deterioration of these materials. Hence, the PET FTIR bands were compared before and during the photo-reforming process and the results were compared with the reported values. For the PET, five main peaks were identified at wavenumbers 731, 1050 to 1100, 1408, 1725, 2907, and 2970 cm⁻¹, corresponding to C—H bending ethyl, C—O ester, C—C phenyl ring, C═O ester, H—C═O group, and C—H ethyl stretching vibration modes of PET. The used PET bands had an overall agreement of 99.46% with reported values of PET bands solidifying the reliability of the results.

TABLE 9
FTIR spectrum analysis of PET particles.

| | Reported | |
|---|---|---|
| Observed peak | band of | Agreement |
| position (cm⁻¹) | PET(cm⁻¹) | (%) |
| 2963 | 2969 | 99.79 |
| 2371 | 2350 | 99.11 |
| 2104 | 2100 | 99.80 |
| 1956 | 1960 | 99.79 |
| 1713 | 1730 | 99.00 |
| 1573 | 1577 | 99.74 |
| 1503 | 1504 | 99.93 |
| 1452 | 1453 | 99.93 |
| 1338 | 1342 | 99.70 |
| 1241 | 1240 | 99.91 |
| 1094 | 1096 | 99.81 |
| 1015 | 1050 | 96.55 |
| 968 | 972 | 99.58 |
| 872 | 872 | 100.00 |
| 847 | 848 | 99.88 |
| 791 | 795 | 99.49 |
| 721 | 712 | 98.75 |

| | |
|---|---|
| Overall agreement | 99.46 |

The IR band for carbonyl (C═O) stretching mode occurred at 1725 cm⁻¹ and broadening of the carbonyl band toward lower wavenumbers was observed (such as 1675 cm⁻¹) due to the formation of the terminal carboxylic acid group. 1245 cm⁻¹, 1125 cm⁻¹, 1340 cm⁻¹, 975, and 845 cm⁻¹ bands were responsible for the various vibration modes corresponding to trans conformer at carbonyl groups and the benzene ring, ether group, and methylene group in PET. Further, the bands at 1370 cm⁻¹, 1096 cm⁻¹, 1040 cm⁻¹, and 900 cm⁻¹ were due to various vibrations corresponding to the gauche conformer of the ethylene glycol unit. These IR bands were indicators of degradation as both UV light and moisture directly attacked these specific chemical bonds and caused chain scissions and changes in the chain conformation and morphology. The IR band at 975 cm⁻¹, was due to asymmetric stretching of the trans-oxy-ethylene (O—CH₂) group in the ethylene glycol unit, and was specifically examined as an indicator of changes in the morphology by transitioning from gauche to trans conformer. Furthermore, broadening below the carbonyl band at 1714 cm⁻¹ and the formation of a new peak at 775 cm⁻¹ were known to be generated by degradation byproducts.

The hydroxyl group at 2900 cm 1 increased over time as a clear indication of the PET degradation process. The band transmittance decreased at 1714 cm⁻¹, 1245 cm⁻¹, 1100 cm-1, and 870 cm⁻¹, indicating the disappearance of ketones and aromatic nature in PET as a process of degradation. A more significant change can be observed at the 845 cm⁻¹, where the band decrease confirms the weakening of the aromatic structure.

PET degradation under UV and visible light irradiation was carried out with the same amount of catalyst and same PET weight to understand the influence of the visible light over the Au doped TiO₂. The transmittance of most of the bands decreased (1714 cm⁻¹, 1245 cm⁻¹, 1370 cm⁻¹, and 1040 cm⁻¹) with the UV irradiance and the formation hydroxyl group (at 2900 cm⁻¹) confirmed the PET was subjected to the photo-reforming process. However, such significant changes were not present in the PET that was exposed to visible light irradiation with Au/TiO₂. Further analysis of the FTIR spectrums carbonyl index and the vinyl index was conducted. The carbonyl index (ICO) was calculated using:

$${I}_{co} = {A}_{1740} - {A}_{1835} / 0.08 \cdot t , \qquad ( 8 )$$

where A₁₇₄₀ and A₁₈₃₅ are the stretching vibration of the carbonyl group, Absorption (A) at 1740 cm⁻¹ and 1835 cm⁻¹ respectively, are calculated using equation 9, where t is the thickness of the sample (in mm), and T is the transmittance (%) of the band.

$$A = - {log}_{10} T . \qquad ( 9 )$$

The Vinyl index (IV) was calculated using:

$$IV = {A}_{909} / {A}_{2020} . \qquad ( 10 )$$

The ratio of absorbance of the deformation band at 909 cm⁻¹ (A909), and the stretching vibration of the vinyl group (CH₂═CH⁻) at 2020 cm⁻¹ (A2020), were calculated using equation 10.

TABLE 10
Comparison of ICO of PET that was exposed
to UV and visible (Vis) light with Au/TiO₂ catalyst.

| | .A 1740 | A 1835 | Thickness/(mm) | ICO |
|---|---|---|---|---|
| Initial | −1.87547 | −2.00546 | 0.254 | 63.97 |
| UV | −1.91368 | −2.00775 | 0.254 | 46.29 |
| Vis | −1.87964 | −1.92836 | 0.254 | 23.98 |

TABLE 11
Comparison of IV of PET that was exposed to UV
and visible (Vis) light with Au/TiO₂ catalyst.

| | A 909 | A 2020 | IV |
|---|---|---|---|
| Initial | −1.89684 | −2.00387 | 0.95 |
| UV | −1.91794 | −2.00627 | 0.96 |
| Vis | −1.86601 | −1.92584 | 0.97 |

The reduction of the ICO and the IV after UV and visible light irradiation (Tables 10 and 11) showed that the UV and visible light was capable of degrading PET. To further analyze the photodegradation of PET, the ratio of the stretching and bending band areas of different functional groups and their variation with time were taken into account.

TABLE 12
Comparison of band areas (“BA”) with respect to the C—H starching
band area of PET that was exposed to UV light with Au/TiO₂ catalyst

| Time | BA_{C═O}/ | BA_{C—O—C}/ | BA_{O—H}/ | BA_{C═C}/ |
|---|---|---|---|---|
| (h) | BA_{C—H} | BA_{C—H} | BA_{C—H} | BA_{C—H} |
| 0 | 6.59 | 7.67 | 2.59 | 3.11 |
| 12 | 15.69 | 16.37 | 0.55 | 3.36 |
| 24 | 1.13 | 0.77 | 0.32 | 0.37 |
| 40 | 4.78 | 7.90 | 0.62 | 1.09 |
| 60 | 29.32 | 21.58 | 2.06 | 6.80 |

The BAC=C/BAC-H and BAO-H/BAC-H decreased with time while BAC=O/BAC-H and BAC-O—C/BAC-H increased up to 12 hours with rapid decrease after 12 hours (Table 12). In general, all the BAs with respect to BAC-H, slightly decreased during a 40 hour period (Table 12). Therefore, the overall decrease of BA rations proved the disappearance of bands was due to degradation of PET.

### Example 21

Gas analysis from the reactor was performed. In the initial experiments, air bubbles were observed under the plastic surface and inside of the reactor. Gas production was evident with the bubble generation in the reactor. Gas identification and quantification was the next step of the process. A closed reactor was designed to collect the generated gas and a gas chromatograph with a thermal conductivity detector (“GC-TCD”) was used to characterize and measure the gas composition.

The headspace gas of the photo-reforming process was analyzed using a GC-TCD. The profile of the gas composition was analyzed at different time intervals. A major portion of the gas in the head space was O₂ while a significant amount of H₂ was produced in the photo-reforming process. The H₂ was not detected in the initial stages of the reaction process (after 30 minutes, 1 hour, 2 hours, 3 hours, 6 hours), however, H₂ was detected in the gas matrix after the 3 hours interval. The photo-reforming process was carried out and the materials were exposed to the UV light. Two sets of experiments were carried out using pure TiO₂ as the catalyst and Au/TiO₂ as the catalyst.

The peak in the range of 3.5 mins to 4.8 mins represented H₂ gas and the peak between 7.1 to 8.5 minutes occurred due to the presence of O₂ gas in the headspace. The presence of H₂ gas was evidence that the photo-reforming process had taken place successfully in the reaction chamber. The constant amount of O₂ further solidified the PET degradation. The maximum amount of H₂ generation was observed at the 6 h time interval with a volume of 21.4 μmol/g of catalyst. Hydrogen generated gradually increased up to 6 h.

The photo-reforming activity of Au/TiO₂ was compared with TiO₂ catalyst under the same conditions. A pure TiO₂ system did not produce H₂ or produced an untraceable amount of H₂, which verified that Au doping of TiO₂ enhanced photocatalytic activity.

### Example 22

Liquid phase dark deposition method of single atom deposition was performed. Annealed P25 Degussa TiO₂ nano powders at 500° C. were immersed in 100 mL MeOH (50 vol %) solution containing diluted H₂PtCl₆·6H₂O solution in a volumetric flask and sealed. The solution was stirred for 24 hours in the dark. After staying in the dark for 24 hours, the nano powders were then filtered and washed with EtOH and deionized water for 15 min each. Subsequently, the samples were dried in an oven at 120° C.

Liquid phase direct deposition/deposition precipitation method of single atom deposition was performed. Controlled deposition of metal cocatalysts on the semiconductor and/or metal oxide surface under ambient conditions was performed and precursor concentration and reaction time influence the particle size and distribution.

### Example 23

PET was converted into EG and Na₂TP under the photo-reforming process. Valuable byproducts formed after the pretreatment and, subsequently, the photo-reforming step. TPA was identified as one such valuable product that can be extracted from the solution after pretreatment. Alkaline and acidic hydrolysis under different conditions was applied in the pretreatment method, as in Table 13. Then the degradation was analyzed considering weight reduction, amount of generated TPA, and extracted TPA amount from the solution mixture. The weight reduction percentage of the PET after the pretreatment process indicated the hydrolysis efficiency of different pretreatment methods. Alkaline hydrolysis methods, such as KOH and EtOH-induced pretreatment, showed higher PET conversion efficiency compared to acidic hydrolysis, such as H₂SO₄ pretreatment. The highest PET conversion of 92.4% was observed with EtOH-induced pretreatment at 40° C., whereas the PET conversion efficiency reduced to 70.8% when decreasing the temperature to 25° C. Increased KOH concentration increased the PET conversion by 2.8 times and 4.1 times under 25° C. and 40° C., respectively.

TABLE 13
Pretreatment methods and PET conversion

| | Weight | Amount of | Extracted |
|---|---|---|---|
| | reduction | TPA in | TPA amount |
| | percentage | the solution | (mg/g of |
| Pretreatment method | (%) | (mg/L) | PET) |
| 3M KOH at 25° C. | 11.6 | N/A | 0 |
| for 24 hours | | | |
| 10M KOH at 25° C. | 31.6 | 122 | 4 |
| for 24 hours | | | |
| EtOH/H₂O 5% NaOH | 70.8 | 1091 | 16 |
| for 24 hours 25° C. | | | |
| H₂SO₄ at 25° C. | 2.8 | N/A | 12 |
| H₂SO₄ at 25° C. with | 0.0 | 420 | 10 |
| NA₂TP | | | |
| 3M KOH at 40° C. | 13.6 | 230 | 0 |
| for 24 hours | | | |
| 10M KOH at 40° C. | 54.0 | 901 | 12 |
| for 24 hours | | | |
| EtOH/H₂O 5% NaOH | 92.4 | 853 | 32 |
| for 24 hours 40° C. | | | |
| H₂SO₄ 40° C. | 4.8 | 112 | 5 |
| H₂SO₄ 40° C. with | 0.0 | 756 | 4 |
| Na₂TP | | | |

### Example 24

The dissolved TPA content in the substrate after pretreatment was evaluated using the HPLC method. The highest dissolved TPA content was observed when EtOH-induced pretreatment was applied at 25° C. Dissolved TPA content increased in all the solutions with increasing reaction temperature. The highest TPA amount was extracted from pretreatment with EtOH at 40° C., which was 200% higher than the extracted TPA under the same method at 25° C. Higher PET conversion resulted in a higher generation of TPA. The added terephthalate ions when PET was converted using catalyzed H₂SO₄ with Na₂TP resulted in a higher TPA concentration in the solution and extracted TPA. Therefore, the observed results suggested the feasibility of PET conversion and TPA extraction even after 24 hours of pretreatment reaction.

The generated dissolved TPA content and resulting H₂ production were analyzed to understand the statistical correlation between them. Increased TPA content in the solution increased H₂ production since it is an indicator of PET hydrolysis.

### Example 25

Table 14 shows the TPA content in the reaction mixture after the photo-reforming process of 48 h and extracted TPA from the mixture. 0.7% (w/w) Pt/TiO₂ was applied as the catalyst, and a UV mercury vapor lamp was used as the light source. The highest extracted TPA content was detected from the reaction mixture pretreated with the EtOH-induced pretreatment method, which is 55 mg TPA per g PET. However, the highest dissolved TPA content in the reaction mixture was measured when 10 M KOH was applied as a pretreatment precursor, even though the extracted TPA from that solution was 40 mg/g of PET. However, results indicated that 48 hours of photo-reforming successfully converted PET, and a significant amount of TPA was extracted from the solution mixture, which could then be reused in H₂ production or in polymer synthesis.

TABLE 14
TPA extraction after photo reforming
process of PET with Pt/TiO₂

| | TPA in the | Extracted TPA after |
|---|---|---|
| Reaction | solution | 2 days of reaction |
| condition | (mg/L) | (mg/g of PET) |
| 3M KOH | 1711.5 | 40 |
| 10M KOH | 2840.0 | 40 |
| EtOH/H₂O | 2222.5 | 55 |
| 5% NaOH | | |
| 1M H₂SO₄ | 1029.3 | 10 |
| 1M H₂SO₄ | 1125.9 | 70 |
| with NA₂TP | | |

### Example 26

Photo-reforming H₂ evolution process was applied using three TiO₂-based catalysts: Au/TiO₂, Pt/TiO₂, and Zn/TiO₂. Further, Pt/TiO₂ was synthesized with Pt loading percentages of 0.3% (w/w), 0.7% (w/w), 1.5% (w/w), and 6% (w/w). Pt and Au are noble metals with excellent catalytic properties, and composite nanomaterials of Pt/TiO₂ and Au/TiO₂ were identified as effective catalysts in photo-reforming reactions due to their ability to facilitate the necessary redox reactions. The doping percentages were tuned to generate atomic deposition of Pt on TiO₂ and consider synthesizing more economical catalysts. The application of Zn/TiO₂ was to assess the ability of replacing noble metals with low-cost metal cocatalysts. Preparation of Au/TiO₂ was performed following the method discussed in our previous study. The hexachloroplatinic acid solution (H₂PtCl₆, 8 wt. % in H₂O) with a volume of 4.7 μl, 23.5 μl, 62.5 μl, and 250 μl was added to 200 ml of deionized (“DI”) water to obtain 0.3% (w/w), 0.7% (w/w), 1.5% (w/w), and 6% (w/w) Pt doping percentages on TiO₂ respectively.

### Example 27

Zn-doped TiO₂ nanoparticles were synthesized by sol-gel method. A solution containing 160 ml of ethanol (EtOH) and 20 ml of isopropanol was mixed slowly with 20 ml (50 mmol) of titanium (IV) butoxide under sonication. The resultant mixture was slowly added to 100 ml of deionized water containing 0.1 g of zinc nitrate hexahydrate and 2 g of cetyltrimethylammonium bromide (“CTAB”) to form a white precipitate. The system was kept under constant stirring at 80° C. for 4 hours. The excess water was removed by evaporation on the water bath with continuous stirring. The resultant precursor was then dried at 110° C. for 12 hours and then finally calcined at 500° C. for 4 hours in a high-temperature muffle furnace.

### Example 28

Transmission electron microscopy was used for imaging Au/TiO₂, Zn/TiO₂, and Pt/TiO₂. Pt/TiO₂ was further analyzed using a TEM equipped with 3rd-order spherical aberration corrector for the scanning TEM (“STEM”) probe. Software was used for further analysis of the TEM images. The crystalline composition of catalysts was characterized with CuKα radiation (λ=1.54 Å) using an X-ray diffraction system. The bandgap of the catalysts was determined by a full wavelength scan within the range of 190 nm to 1100 nm using a UV spectrophotometer. The Tauc plot method was applied to derive the bandgap energies of the catalysts following our previous study. X-ray photoelectron spectroscopy (“XPS”) equipped with a monochromatized Al K-alpha X-ray source with a photon energy of 1486.6 eV and operated at 15 keV and 20 mA emission current was used to characterize the electronic properties of the catalysts and to determine the loading percentages. The loading percentages were further verified using inductively coupled plasma optical emission spectroscopy (“ICP-OES”) following Environmental Protection Agency (“EPA”) 200.7 method. The ICP-OES analysis was followed by the microwave digestion of the sample following the EPA 3051A method without using HF.

### Example 29

PET was hydrolyzed during a pretreatment process using two methods. During the first method, KOH was used as a precursor at two different concentrations of 3 M and 10 M. KOH solution (10 ml) was added to 0.5 g of finely cut PET and stirred at 300 rpm for 24 hours at two different temperatures (25° C. and 40° C.) on different occasions. After 24 hours, the solution was diluted by adding 40 ml of deionized water, and the photo-reforming process was started after adding the catalyst in a sealed quartz reactor.

When using the ethanol-induced pretreatment method, 6 ml of EtOH was added to finely cut 0.5 g of PET with 4 ml of deionized water and 0.5 g of NaOH was added to the solution and stirred for 3 hours and 6 hours at 25° C.

When testing the influence of the pretreatment processes, the polymer-to-catalyst ratio was maintained at 1:1. Photo irradiation continued for 2 days, and hydrogen production was measured at 6-hour intervals.

### Example 30

A photo-reforming experiment under UV-irradiation was conducted using a high-pressure UV mercury vapor lamp. Light flux was measured using a solar power meter. Gas generated during the reaction was analyzed using a gas chromatograph (“GC”) equipped with a thermal conductivity detector (“TCD”) and HP-5 molecular sieve column using N₂ as the carrier gas as described in our previous study.

When analyzing the effect of pH on hydrogen production efficiency, a 1:1 polymer-to-catalyst ratio was maintained, Au/TiO₂ and Pt/TiO₂ were used as the photocatalysts. Two different tests at pH 7 and existing pH (>13) without adjusting were conducted. The effect of polymer to catalyst ratio was analyzed in the system by testing three different ratios 1:1, 2:1, and 1:2. When changing the polymer-to-catalyst ratios, pH remained constant at >13 without further adjustment, and two different pre-treatment conditions with two catalysts were tested. Each of the above tests was continued for 48 hours with a duplicate under irradiation of a high-pressure UV mercury vapor lamp, and hydrogen production at 18 hours, 24 hours, 30 hours, 42 hours, and 48 hours was measured.

### Example 31

Computational simulation of the photocatalytic activity of the catalysts was performed. Density functional theory (“DFT”)-based calculations were performed. The lattice parameters of DFT periodic boundary calculation of anatase TiO₂, Au/TiO₂, and Pt/TiO₂ were a=3.800 Å and c=9.708 Å. The surface of TiO₂ and composite materials structures were modeled using a (2×2) supercell. The supercell contained four Ti layers and twice as many O layers in a slab, with 5 Å vacuum separating the surface slabs. In addition, in metal composite material, an Au or Pt atom was placed on the Au/TiO₂ and Pt/TiO₂ surface DFT calculation, respectively, of the TiO₂ surface.

The initial structures were optimized using Perdew-Burke-Ernzerh of nonlinear core-correction Rappe Rabe Kaxiras Joannopoulos (“PBE-n-rrkjus”) and pseudopotential plane wave approach. The calculations were carried out using plane wave functions with a kinetic energy cutoff of 30 Ry and a 60 times larger cutoff for charge density with a k-points grid of 8×8×8. During the optimization, two bottom Ti layers and corresponding O atoms were fixed until the forces acting on atoms were less than 0.001 eV Å-1 in any direction to reduce the complexity of the calculation. These optimized structures were used to obtain the Density of states (“DOS”) for further structural analysis.

### Example 32

XRD analysis was performed on the nanocomposite catalysts. X-ray diffraction analysis of TiO₂, Pt/TiO₂, and Au/TiO₂ was performed to identify crystalline changes because of doping Au and Pt. Compared with pristine TiO₂, there were few changes in the distribution and intensity of XRD peaks in Pt/TiO₂ and Au/TiO₂. All the indexes showed in TiO₂ can be observed in XRD patterns of Pt/TiO₂ and Au/TiO₂ which indicated that the crystalline structure of the TiO₂ was not affected by the chemical precipitation process of Au and Pt doping. The Au (111) lattice index overlaps with the TiO₂ Anatase (103) lattice index, which confirmed the surface deposition of the Au on TiO₂. However, there were no significant changes in the XRD patterns due to smaller particle sizes and minimum doping amounts of metal on TiO₂. The characteristic peak of the Pt (111) lattice index was visible at two theta values of 38.9° in 6%, 1.5%, and 0.7% Pt/TiO₂ XRD patterns but imperceptible in 0.3% Pt/TiO₂ XRD spectra due to undetectable level of Pt.

### Example 33

XPS analysis was performed on the nanocomposite catalysts. The elemental composition was determined by XPS high-resolution scans over C 1s, O 1s, Ti 2p, and Pt 4f spectra regions. The XPS analysis was performed to investigate the electronic structure of the elements (Pt, Au, Ti, and O) on the surface of catalysts before and after the synthesis. The energy scale was corrected to a binding energy of Ti 2p at 458.5 eV and the referenced C 1s peak at 284.6 eV for surface adventitious carbon. The peaks at 72.0 eV, and 76.0 eV are responsible for Pt 4f7/2 and Pt 4f5/2, respectively. Furthermore, peak deconvolutions indicate the peak positions at 72.6, 74.0, 76.1, and 77.8 eV, corresponding to the presence of Pt²⁺ and Pt⁴⁺ species which may exist in the form of PtO and PtO₂ on the surface of the catalyst. However, the calculated ratio of Pt⁰/Pt²⁺ concerning peak areas is 5.9, indicating that the Pt⁰ species are dominant compared to Pt²⁺ on the surface of the catalyst. The oxidation state of Pt is a crucial factor when improving photocatalytic efficiency. Even though the bandgap energy is higher in PtO/TiO₂ compared to Pt/TiO₂, better hydrogen production via water-splitting was observed in the presence of PtO/TiO₂.

### Example 34

A KOH polymer pretreatment method was performed. Different pretreatment methods were applied to enhance the hydrolysis of PET particles. KOH concentration and the pretreatment temperature were considered key parameters that affect the hydrolysis efficiency. Two KOH concentrations of 3 M and 10 M were experimented with temperatures at 25° C. and 40° C. Hydrogen production concentration in the PET hydrolysis process since the catalytic efficiency was dominant compared to polymer hydrolysis efficiency. However, there was a slight increase in hydrogen production using Zn/TiO₂ as the catalyst when increasing the KOH concentration in the pretreatment process. When using Au/TiO₂ as the catalysts, hydrogen production with 10 M KOH pretreated PET solution was about 3.5 times the H₂ production with 3 M pretreated PET. However, H₂ production with 10 M KOH was 1.2 times compared to 3 M KOH pretreatment at 25° C. when using Pt/TiO₂ as the catalyst.

### Example 35

An EtOH-induced polymer pretreatment method was performed. The EtOH-induced pretreatment process was applied to enhance the hydrolysis of the PET particles such that more ethylene glycol yield could be produced, which acts as the sacrificial agent during the photo-reforming process. During the pretreatment process, the temperature remained constant at 25° C., and 5% NaOH was added to promote the reaction and to achieve a higher conversion yield of monomers. A mixture of 60% EtOH and 40% deionized water was found to be the most efficient ratio as a co-solvent in PET hydrolysis. The photo-reforming results indicate that longer pretreatment time increased hydrogen production by 30% and 15% with Au/TiO₂ and Pt/TiO₂, respectively. Increasing the pretreatment time could increase PET monomer yield, thus resulting in higher H₂ production.

### Example 36

The polymer-to-catalyst ratio was evaluated. When comparing the H₂ production concerning polymer-to-catalyst ratios, the highest H₂ yield was observed when using Pt/TiO₂ as the catalyst at a ratio of 2:1, where 0.5 g of pretreated PET was mixed with 0.25 g of the catalyst. Increasing the catalyst dosage from 0.25 g to 1.0 g resulted in a 37% and 64% reduction in H₂ yield with Pt/TiO₂ and Au/TiO₂, respectively. High catalyst density in the slurry system might act as an obstacle to lower light penetration, resulting in lower photocatalytic efficiency. Therefore, decreasing catalyst dosage (for both Au/TiO₂ and Pt/TiO₂) led to an increase in H₂ production in the EtOH-pretreated PET system.

The Pt/TiO₂ to PET ratio in the KOH-pretreated system behaved as an analog to Pt/TiO₂ EtOH-pretreated PET system, showing higher H₂ production at the 2:1 polymer-to-catalyst ratio. However, for Au/TiO₂ KOH-pretreated system, the highest H₂ yield was achieved at a 1:1 polymer-to-catalyst ratio. The lowest H₂ yield (˜183 μmol/g_{cat}) was observed when 1.0 g of Au/TiO₂ was added with 0.5 g of KOH-pretreated PET. Increasing the catalyst dosage (by a factor of 2) increased the H₂ yield (by ˜1.4 times) but further increasing catalyst dosage beyond the optimal ratio reduced hydrogen production due to inhibited light absorption and utilization.

### Example 37

Pretreatment of plastics was performed. PET, LDPE, and PS were collected from waste plastic beverage bottles (the average molecular weight was 24,800 g/mol), low-density polyethylene bags, and polystyrene plates, respectively. The plastic materials were thoroughly washed with deionized water and ethanol (EtOH) and air-dried before pretreatment. Three different pretreatment methods were applied to acquire the monomers and oligomers of the plastics. KOH was used to promote hydrolysis by adding 0.5 g of finely cut plastic into 10 ml of 3 M KOH solution and stirring for 24 hours. The ethanol pretreatment process applied ethanol (EtOH) and deionized water in a 3:2 ratio, with 5% (w/w) NaOH used as the precursor with 0.5 g of finely cut plastic in 10 mL solution (EtOH-induced pretreatment method). Acidic hydrolysis was applied, where 0.5 g of finely cut plastic was added to 10 mL of 1M H₂SO₄. The mixture was stirred at 25° C. for 24 hours, and the pH of the resulting supernatant was adjusted to 7 using 10 M KOH solution.

### Example 38

PET hydrolysis was studied under two major processes, including alkaline and acidic pretreatment. The applicability of PET as a sacrificial agent was further studied by optimizing the pretreatment under five different pretreatment conditions. An additional alkaline hydrolysis method was applied using a 10M KOH solution. Further, the acidic hydrolysis was catalyzed by adding 0.01 g of Na₂TP to the solution. These pretreatments were applied into 0.5 g of finely cut polymer with 10 mL of total solution at 25° C. for 24 hours. Byproduct formation under different PET pretreatment conditions was quantified by high-performance liquid chromatography analysis, and the residuals were characterized using ATR-FTIR analysis and TGA after each pretreatment and after photo-reforming process.

### Example 39

The EtOH-induced pretreatment process was studied due to its higher H₂ production ability and higher hydrolysis rate under mild conditions. The pretreatment process was performed in five steps. The baseline study was conducted using 30 ml of absolute EtOH mixed with 20 ml of deionized water as the first step. The finely cut PET 0.5 g was added to the EtOH and deionized water mixture, followed by 5% (w/w) NaOH, and pretreated for 24 hours. The resulting mixture was filtered with 0.1 μm filters, and the filtrate was used in the second step of the photo-reforming experiment. In the third step of the experiment, the same mixture was used without filtering. The supernatant extracted during the second step was dissolved in 50 ml of deionized water and used in the 4th step. The PET contained EtOH and deionized water mixed with 5% (w/w) NaOH 24 hours pretreated solution was exposed to dropwise addition of 1 M H₂SO₄. The resulting white precipitate was extracted by centrifugation and used in the photo-reforming experiment during the 5th step after suspending in DI water. Each photo-reforming experiment was conducted after adding 0.5 g of the catalyst, adjusting the total volume of the reactor to 50 ml, and a 60 mW/cm² UV mercury vapor lamp was used for irradiation.

### Example 40

Photo-reforming experiments were performed in sealed 50 ml quartz reactors under simulated light of a high-pressure UV mercury vapor lamp with minor peaks at 290 nm, 315 nm, 335 nm, and a dominant peak at 365 nm. The flux of the light source was measured using a solar power meter as 60 mW/cm². A volume of 10 ml of pretreated polymer solution was added to the reactor with 0.5 g of the catalyst. The preparation and characterization of Au/TiO₂ and Pt/TiO₂ nanocomposite photocatalyst were discussed in our previous study. H₂ production was measured using a gas chromatograph (“GC”) equipped with a thermal conductivity detector (“TCD”) and a molecular sieve column using N₂ as the carrier gas and 1 mL of headspace gas was directly injected into the inlet of the gas chromatography. Purge time was selected as 1 min, and chromatography was collected for 10 mins after injection. The sample peak retention time (appearance time) and area were compared to standard H₂ samples to calculate the gas concentration.

### Example 41

Polymer degradation was analyzed by comparing ATR-FTIR of residual plastics after pretreatment and photo-reforming process. Plastic pieces were washed with deionized water and dried before acquiring the ATR-FTIR spectrum. The polymer degradation was reflected by the formation of carbonyl group (C═O) hence the carbonyl index was an indicator used to measure the chemical oxidation of polymers. The carbonyl index (CO index) was calculated using Equation 11 and compared with that of virgin (non-degraded) PET. The CO index was calculated from the ratio between the integrated band absorbance of the carbonyl (C═O) peak from 1,850 cm⁻¹ to 1,650 cm⁻¹ and that of the methylene (CH₂) scissoring peak from 1,500 cm⁻¹ to 1,420 cm⁻¹.

$${I}_{co} = \frac{Area under band 1 , 850 - 1 , 650 {cm}^{- 1}}{Area under band 1 , 500 - 1 , 420 {cm}^{- 1}} \qquad ( 11 )$$

### Example 42

TGA was performed to evaluate the degradation process and decomposition potential after pretreatment of residual PET particles. Pt pans for furnaces were loaded with the washed and dried residual PET particles (˜10 mg) filtered after the pretreatment process after taring the small Pt pans. A carrier gas (N₂) was applied at a flow rate of 40 mL/min in the temperature range from 30° C. to 800° C. with a heating rate of 20° C./min. Related weight loss bends, plotting, and analysis were completed using resulting TGA curves.

### Example 43

High-performance liquid chromatography (“HPLC”) equipped with a column was employed to analyze the liquid byproducts formed after pretreatment and photo-reforming of PET. The UV/vis detector was set at 256 nm, and the reverse phase C18 column was used. A methanol/water 80/20 (v/v) was used as the mobile phase at a flow rate of 1 ml/min and at injection sample volume of 10 μl. Terephthalic acid was identified and quantified using the calibration with standard TPA (benzene-1,4-dicarboxylic acid).

### Example 44

PET weight reduction was evaluated after pretreatment. PET weight reduction after pretreatment was analyzed using 0.25 g of initial weight (“W₀”) of PET in a tared glass vial. Then 5 ml of solution was added and pretreated for 24 hours. The reaction mixture was filtered with a 0.5 mm sieve after 24 hours, and residual PET particles were washed with deionized water. The resulting residual particles were oven-dried at 30° C. for 24 hours, and the final weight (“W_{f}”) was recorded. The reduced weight percentage was calculated using Equation 12.

$$Reduced weight percentage = \frac{( {W}_{0} - {W}_{f} )}{{W}_{0}} \times 100 \% \qquad ( 12 )$$

### Example 45

TPA extraction was performed. The filtrate after the pretreatment was collected and 0.5 ml of 1 M H₂SO₄ was added dropwise to the solution to precipitate the dissolved terephthalate ions to TPA. The resulting solution was stirred at 300 rpm for 20 mins and vacuum filtered using tared 0.1 μm filters. The filtered suspension was air-dried for 48 h at room temperature and weighed. The weight of the extracted TPA amount was the increased weight of the filter.

The preceding examples can be repeated with similar success by substituting the generically or specifically described reactants and/or operating conditions of this invention for those used in the preceding examples.

Note that in the specification and claims, “about” or “approximately” means within twenty percent (20%) of the numerical amount cited. The terms, “a”, “an”, “the”, and “said” mean “one or more” unless context explicitly dictates otherwise.

Although the invention has been described in detail with particular reference to these embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited above are hereby incorporated by reference.

What is claimed is:

1. A composition for producing hydrogen comprising:
a semiconductor; and
a plurality of metal atoms attached to a surface of said semiconductor, wherein each of said plurality of metal atoms is not in contact with another metal atom attached to said surface of said semiconductor.

2. The composition of claim 1 wherein said plurality of metal atoms comprises a platinum group metal.

3. The composition of claim 2 wherein said platinum group metal comprises platinum.

4. The composition of claim 1 wherein said plurality of metal atoms comprises a precious metal.

5. The composition of claim 4 wherein said precious metal comprises gold.

6. The composition of claim 1 wherein said plurality of metal atoms comprises zinc.

7. The composition of claim 1 wherein said semiconductor comprises a metal oxide.

8. The composition of claim 7 wherein said metal oxide comprises titanium dioxide.

9. The composition of claim 1 wherein said semiconductor comprises silicon dioxide

10. A method for producing hydrogen, the method comprising:
pretreating a polymer, wherein pretreating the polymer comprises contacting the polymer with a first aqueous solution;
contacting the polymer with a second aqueous solution;
contacting the polymer with a nanocomposite, the nanocomposite comprising:
a semiconductor; and
a metal attached to a surface of the semiconductor;
contacting the nanocomposite with radiation;
oxidizing the polymer; and
forming hydrogen.

11. The method of claim 10 further comprising forming disodium terephthalate.

12. The method of claim 10 further comprising forming terephthalic acid.

13. The method of claim 10 wherein the polymer comprises polyethylene terephthalate.

14. The method of claim 10 wherein the polymer comprises low-density polyethylene.

15. The method of claim 10 wherein the polymer comprises polystyrene.

16. The method of claim 10 wherein pretreating the polymer comprises contacting the polymer with a base.

17. The method of claim 10 wherein pretreating the polymer comprises contacting the polymer with ethanol.

18. The method of claim 10 wherein pretreating the polymer comprises contacting the polymer with an acid.

19. The method of claim 10 further comprising contacting a metal with a semiconductor in the absence of light to form a nanocomposite.

20. The method of claim 10 further comprising adjusting the pH of the second aqueous solution.
